(19) United States
(12) Patent Application Publication
(10) Pub. No.: US 2025/0195809 A1
(43) Pub. Date: Jun. 19, 2025

(54) A MASK FOR DELIVERING PRESSURISED AIR TO A USER

(71) Applicant: Breas Medical Pty. Ltd., Macquarie Park, NSW (AU)

(72) Inventors: Philip Kwok, Sydney (AU); Christopher Dodds, Sydney (AU); Benjamin Lawrence, Sydney (AU); Andrew Hung, Sydney (AU); Eric Siu, Sydney (AU); Kirrily Haskard, Sydney (AU); Toby Wheeler, Sydney (AU); Alaitz Izaguirre, Sydney (AU); Albert Chau, Sydney (AU); Jake Harris, Canberra, ACT (AU); Yukai Fu, Sydney (AU); Charlotte Blankensteijn, Amsterdam (NL); Daniel Kyle Pasqualino, Sydney (AU)

(73) Assignee: Breas Medical Pty. Ltd., Macquarie Park, NSW (AU)

(21) Appl. No.: 18/978,880

(22) Filed: Dec. 12, 2024

(30) Foreign Application Priority Data
Dec. 14, 2023 (AU) 2023904054
Feb. 16, 2024 (AU) 2024900379

(51) Int. Cl. A61M 16/06; A61M 16/08; A61M 16/20

(52) U.S. Cl. CPC A61M 16/0616; A61M 16/0683; A61M 16/0875; A61M 16/20

(57) ABSTRACT

A mask adapted for delivering pressurised air to a user, the mask comprising: an endoskeleton of a first resilient material encapsulated to a lamina of a second resilient material forming a mask body with a cavity. The second resilient material is more flexible than the first resilient material. The lamina having a face contactable skirt portion extending away from the endoskeleton perimeter. The skirt portion comprises an outer layer and an inner layer for providing variable contoured thickness to outer lamina segments of the face contactable portion. The face contactable skirt portion having a flexible cavity edge defining a cavity opening for receiving a nasal region. The endoskeleton and lamina has an aperture adapted to connect to an air delivery tube; the thinner flexible cavity edge is adapted to wrap into the cavity, when wrapped, the contoured thicker lamina segments wraps and seals around the sides of the nose.

### TECHNICAL FIELD

The present invention relates to the field of respiratory devices, specifically masks and/or a mask assemblies for use in delivering pressured air to a user. More particularly, the invention relates to improvements in the design and functionality of masks utilised in various non-limiting applications such as medical treatments, respiratory therapy and sleep apnoea management.

### BACKGROUND

Respiratory masks have been indispensable in the medical and therapeutic fields, offering vital support for individuals with respiratory conditions or those requiring supplemental oxygen therapy. Amongst these devices, continuous positive airway pressure (CPAP) masks play a significant role in maintaining airway patency during sleep and enhancing respiratory efficiency. Conventional CPAP masks typically comprise a mask body, headgear for securing the mask to the user's face and an air supply system connected to the mask. While existing masks have been developed, there remains a need for improvements that address limitations or non-considered benefits in terms of user comfort, ease of use, and adaptability to diverse facial structures and force distribution. Further challenges associated with conventional masks involve issues with positive air leakage, discomfort during prolonged use, and difficulty in achieving an effective seal on different and complex facial shapes. Additionally, conventional mask designs also lack versatility in adapting to the dynamic movements and positions of users during sleep.

Conventional masks have fit and seal issues and have trouble in maintaining a proper seal between the mask and the user's face. As there is no average face, there is no average mask. Facial contours vary widely, and conventional cushions struggle to provide a secure and leak-free seal for all users. Additionally, mask deformation from the compressive forces can stretch the materials and become taut and in so doing causing cuts and/or injury to the user's face. While most conventional mask assemblies may focus on the cushion, headgears force distribution are less explored and the headgear can create pressure points on the face and head, leading to discomfort and even headaches. Further, straps on the CPAP headgear can tangle and biased backwards leading to users overstretching or straining their arms in trying to find and untangle headgear straps to put onto the mask. Further, where magnetic headgear clips are used in some conventional headgears, the lower strap ends can inadvertently attach together behind the head exacerbating the poor useability issue. Further conventional elbows for air delivery are also noisy when air is coming out from the valves, and so can be disruptive to a neighbouring person's sleep. As such, there is a long felt need to provide vent hole shapes that not only does what's intended but also to greatly minimise sound.

Any discussion of the prior art throughout the specification should in no way be considered as an admission that such prior art is widely known or forms part of common general knowledge in the field.

### SUMMARY

### Problems to be Solved

It may be an advantage to provide an endoskeleton-cushion module that can accommodate and seal against different face shapes and reduce unintentional air leaks and blowout.

It may be an advantage to provide stiffer membrane and/or thickened ribs, and an extended edge/extended wings at the nose bridge in the cushion region or the face contactable skirt portion so as to minimise blow out at the nose bridge.

It may be an advantage to provide a narrow nose contour or a biased contour to fit smaller and flatter noses.

It may be an advantage to increase the surface area at the sides of the nose bridge for improving the seal for persons with longer nose contact length.

It may be an advantage to provide nose wings at the sides of the nose region of the cushion so as to reduce positive pressure air blow out at this region.

It may be an advantage to provide vertical corrugation ribs at the sides of the nose bridge region of the cushion for allowing easy expansion and contraction of the membrane/lamina/layer to accommodate wider facial geometries.

It may be an advantage to provide a stress relief radius at the nose bridge centre region for allowing cushion membrane to expand easier for larger noses.

It may be an advantage to position a thin membrane behind a thick membrane at the nose bridge side and the nose bridge centre region to allow the thin membrane to reduce pressure from the thick membrane in the event of significant cushion compression.

It may be an advantage to provide an inner secondary seal membrane tailored to different known challenging nose geometries, in which the inner secondary seal membrane engages the nose for an improved seal. The secondary seal membrane can advantageously clamp the thin seal membrane after compression for better sealing.

It may be an advantage to provide a thick polished edge around the edge of the cushion for an improved seal around the edge or cavity edge. It may be a further advantage to provide targeted polished areas to the face contactable skirt portion so to improve seal contact without fully sacrificing comfort.

It may be an advantage to provide a frosted or matte finish around portions of the cushion contact region for an improved slip against the skin to reduce stretching and pressure sores while maintain an effective seal.

It may be an advantage to provide a small localised dual wall only at the sides of the nose to improve seal around the nose. The advantage of localising them at the sides of the nose means that cheek compression does not affect their performance.

It may be an advantage to provide a thin membrane at the cheek edge of the cushion as stiffness is not required right at the edges so the membrane can be thinned earlier to resolve air leakage from the mouth region. It may be a further advantage to extend the thin membrane inwards to increase the compliant section of cushion membrane to reduce air leakage from the mouth region.

It may be an advantage to provide stiffening ribs under the skirt portion at the cheek region to reduce air blow out from the cheek. The stiffness of the stiffening ribs can be adjusted for stiffness under predetermined compression pressures.

It may be an advantage to provide a curled or wrapped membrane instead of a folded membrane as curled or wrapped membranes are more likely to roll as opposed to folded membranes which are more likely to bend or crush.

It may be an advantage to provide an inner membrane that can be a cantilever beam design for conveniently generating the bespoke variable thickness to the skirt portion region.

It may be an advantage to provide a variable thickness inner wall for combining the membrane performance of current variable wall thickness cushion membranes with the added compliance and seal benefit of a thin seal outer wall. It may be a further advantage to provide an outer wall membrane or layer that could be frosted for user comfort.

It may be an advantage to provide a simple multi segment dual wall within the cushion to decouple the stiffness in different areas of the cushion allowing for a more personalised fit.

It may be an advantage to provide a complex multi segment dual wall within the cushion, in which the complex version has relatively more segments compared to the simple version, in which the complex version increases the ability for the cushion to adapt to unique face shapes.

It may be an advantage to provide a chin gimble for increasing the compliance for jaw drop to reduce positive air leakage from the jaw. The chin gimble is a mechanism or structure in the mask that helps maintain proper jaw positioning, especially when the wearer experiences a ‘jaw drop’ or an involuntary or unconscious relaxation of the jaw muscles. By stabilizing the jaw, it advantages forms a better seal to the mask and prevents leaks and improving efficacy.

It may be an advantage to provide an extended chin membrane to also increase the compliance for jaw drop to reduce positive air leakage from the jaw.

It may be an advantage to provide a chin gimble for increasing the compliance for jaw drop and to reduce the tendency to push the jaw open leading to increased mouth breathing.

It may be an advantage to provide an endoskeleton made from rigid, semi-rigid or flexible plastic, or a combination of, for applying pressure to the cushion membrane edge or providing structural integrity to the cushion to control leaks.

It may be an advantage to provide a cushion membrane that varies in stiffness around the edge which varies the force it applies to the face in these areas, which in turn varies the force required by the endoskeleton to these areas without impacting cushion performance.

It may be an advantage to vary the shape of the endoskeleton to accommodate for different nose and face shapes.

It may be an advantage to provide a cushion module that can seal below the sellion and provide a narrow cushion opening to seal against narrow noses.

It may be advantage to provide the mask or endoskeleton-cushion module a general avocado shape so that when the cushion rolls in, the surface is flatter to seal against the skin of the person. Avocado shape may mean in this specification, a general shape with a rounded spherical portion integrally moulded with a relatively thin elongate rounded neck portion extending away from the spherical portion in a manner that would be generally consistent with the general shape of an avocado or pear and more particularly wherein the spherical portion is has an increased width or diameter relative the neck portion.

It may be an advantage to provide a cavity width between the left side and the right side of the nose of a predetermined width that can interfere with the widest nose width but flexible enough to curl away.

It may be an advantage to allow a headgear load to transfer vertically into the side of the mouth and reduce horizontal component of headgear force.

It may be an advantage to have a predetermined width and shape between the left and the right bottom sides of the mask to fit majority of mouth widths and without irritating the lips.

It may be an advantage to have the cushion or the face contactable skirt portion with a sufficient depth to allow good cushioning to the face of the user.

It may be an advantage to have a sufficient height to not put undue pressure on the chin as well as to contact chin earlier and allow for mouth movement. This sufficient height renders a ‘V’ profile shape from the bottom view of the mask, in which this ‘V’ profile shape allows the membrane or the skirt portion to contact the chin earlier.

It may be an advantage to have a sufficient height to not put undue pressure on the chin in a direction that promotes the opening of the mouth encouraging undesirable mouth breathing.

It may be an advantage to provide the distal end of the membrane to be biased so that it will readily contact the skin and form a good seal.

It may be an advantage to provide a thin layer area to conform to different sizes of nose bridge or sellion of the user.

It may be an advantage to provide a thin ‘flap’ or extended nose wing to conform to various nose geometries and to allow air pressure to push the ‘flap’ against the nose.

It may be an advantage to provide a gradual transition between the thickness areas of the cushion so that the membrane or layer does not fold and provides a controlled rolled contacting surface to improve dynamic seal.

It may be an advantage to provide a thin layer area to reduce force on the chin region and to accommodate mouth movement and reduce the risk of cutting the chin region of the user.

It may be an advantage to provide a lower mouth corner rather than positioning with the mouth corner so that it lines up with the mouth better and not irritate the lips of the wearer.

It may be an advantage to provide a local thickness at the side of nose to direct force normal to the side of the nose or to hug the nose under positive air pressure. The thickness may be a spiral shape so as the force can travel along the nose portion of the cushion and flex away if the force is too high.

It may be an advantage to provide part lines at the endoskeleton perimeter separating the endoskeleton and the cushion or the face contactable skirt portion to reduce change in curvature. It may be an advantage to provide force deflection curves to be similar across thick regions.

It may be an advantage to provide an endoskeleton with a major segment with a shape that limits the amount of cushion movement up and out from the desired areas of the mask.

It may be an advantage to provide a silicone band positioned at the endoskeleton perimeter of the minor segments, in which the silicone band acts as a tensioner to transfer the force in alternative directions.

It may be an advantage to provide an encapsulated endoskeleton so that it centres the endoskeleton within cushion wall thickness, and it also prevents the cushion from blowing out and helping to keep the cushion in position.

It may be an advantage to provide a minor segment which has a shape formed from the cutaway segments of the endoskeleton, in which the cutaway segments allow the cushion to flex up and/or out to accommodate different cheek geometries.

It may be an advantage to provide an encapsulated endoskeleton that can direct load onto the cushion with a wider region to anchor the cushion to side of the user's mouth.

It may be an advantage to provide thin areas to allow the cushion assembly to flex.

It may be an advantage to provide an endoskeleton has a shape that could be combined with the cushion geometry to get the right performance.

It may be an advantage to provide an endoskeleton made with different resilient and relatively rigid materials to get the desired flexibility and/or stiffness for the prescribed positive air pressure when in use.

It may be an advantage to provide an endoskeleton option that is made with customised regions resilient and relatively rigid materials to get the desired flexibility and/or stiffness for a specific therapeutic use such as high pressure non-invasive ventilation.

It may be an advantage to provide an exoskeleton mountable to the encapsulated endoskeleton, in which the exoskeleton has an upper frame and a lower frame for securing the upper headgear portion and the lower headgear portion respectively.

It may be an advantage to provide vertically corrugated sections along the elongate upper frame for providing an optimal flex.

It may be an advantage to provide a vertically corrugated sections which end before the upper frame turns upward so as to reduce unwanted flexing under headgear tension.

It may be an advantage to provide a longer vertically corrugated section and bigger pitch at the upper frame to hug the face better and also using less external force to break seal in user's dynamic movements.

It may be an advantage to provide a bigger corrugation pitch along a longer distance along the upper frame arm so as to provide a more gradual bending, better force transfer, and perform less like a hinge.

It may be an advantage to provide an endoskeleton cushion module for the nasal region only in which the endoskeleton has a major segment of a resilient rigid material to provide rigidity to regions of the nose where needed, and the minor segments are of a resilient flexible material to provide flexibility and comfort and a wider fit range.

It may be an advantage to provide a headgear which has a lower back-neck raised to improve comfort and decrease headgear tension variation during use when the user is wearing it and moving around. The lower strap of the headgear advantageously keeps its shape to be biased forward to avoid loss of strap behind neck to allow less nimble users less chance of injury.

It may be an advantage to provide an exoskeleton with frame arms on both sides, in which each frame arm has a vertically corrugated section between the exoskeleton connection and the headgear strap connections. The corrugated geometry allows good flexing around face for comfort whilst still maintaining the strength to stabilise the mask.

It may be an advantage to provide the frame arm to have a bifurcated portion to branch the frame arm to an upper frame arm and a lower frame arm to secure to the upper headgear strap and the lower headgear strap for balancing the upper and lower forces and maintaining the mask to the user face and reduce the instance of rotating off the face of the user.

It may be an advantage to provide a bevelled hook clip at the lower frame arm for connecting to the lower headgear connector for convenient and secure engagement while also providing an easy way for a user to disengage when wanting to remove the headgear from the mask by rotating the headgear strap connection to come off.

It may be an advantage to provide an endoskeleton cushion module for the nose region only that can support sides of the nose bridge to prevent positive air pressure blowout and/or leakage from the user's nasal area.

It may be an advantage to provide a seal membrane or a minor segment of a flexible material (which is absent of the rigid endoskeleton material) at the nose bridge region for providing more comfort to the user and increasing and improving the compliance for people with deep nose bridges.

It may be an advantage to provide a rigid endoskeleton material to the bottom corners so as to apply load just above the lip to iron out the crease of the face to achieve optimal contact and seal.

It may be an advantage to provide an upper frame arm that has a solid, rigid section positioned between the vertically corrugated section and the upper headgear connector, in which the solid, rigid section allows the upper frame arm to stop twisting and maintain the shape and strength when under tension from the headgear.

It may be an advantage to provide an upper headgear connector which has an open loop for easy headgear assembly and disassembly, which allows no readjustment of user strap position required so as to not disrupt user settings.

It may be an advantage to provide a corrugated geometry to flex around face for additional comfort.

It may be an advantage to provide a headgear with simple loops to hook onto the upper/lower frame arm, and can be easily detached by unhooking as loop comes in or detaches when user pulls down at strap.

It may be an advantage to provide a raised lip edge of the cushion or face contactable skirt portion for when this edge stretches, this edge will not be too taut to avoid cutting into the top lip. Instead, the inner layer or surface underneath the skirt portion will be stretched taut as the bottom corner regions flex outwardly from the positive air pressure.

It may be an advantage to provide an endoskeleton that has a shape designed to transfer, absorb or dampen the different forces generated between the cushion and the skin and to always ensure good sealing contact with the skin of the user.

It may be an advantage to provide a default variation of the endoskeleton shape to accommodate average faces, and it may be an advantage to allow for variations in endoskeleton shape to accommodate unique face geometries be it the endoskeleton shape having wider or slimmer structural arms for providing more structural support to larger or smaller cushion regions respectively, and/or providing a wider elbow bore ring to increase more flexible segments, and/or providing shorter structural member compared to the default variation for increasing more flexible segments for added comfort.

It may be an advantage to provide a mechanical interlock variation for strengthening the connection between parts of the mask.

It may be an advantage to provide a nylon frame for the exoskeleton as it is lightweight, durable and flexible without breaking making them more forgiving in situations where the frame will be subject to stress or impact. The flexibility also contributes to the overall comfort of the user. Nylon is also hypoallergenic, making it an excellent choice for individuals with sensitive skin or allergies so that the user will not experience skin irritation.

It may be an advantage to provide an exoskeleton having a structural ribs to add stability to the exoskeleton, as well as snaps positioned around the periphery of the exoskeleton aperture so as to connect with the endoskeleton when mounting the exoskeleton over the front of the endoskeleton such that the exoskeleton aperture lines up congruently to the endoskeleton aperture.

It may be an advantage to provide a frame arm that have an open ‘C’ profile for ease of upper headgear strap attachment or removal.

It may be an advantage to provide a slot for a smaller angle (±20°) of angle articulation for increased stability and less sliding motion between the exoskeleton surface and the frame arm surface. It also requires less space to achieve a range of motion where space is limited allowing for more compact and efficient designs.

It may be an advantage to provide a wave geometry that matches the curvature of the face so that the frame arm can twist in the correct direction.

It may be an advantage to provide an improved clip interface with a ‘click’ engagement for improving the securing means.

It may be an advantage to reduce the perception of the bulk on the face and move the top buckle forward away from the ear, all for improving the comfort of the user.

It may be an advantage to provide a no wave or no corrugated section for the frame arm which in certain face geometries, maintain flexion against the face, and to reduce or eliminate torsion of the upper frame arm.

It may be an advantage to provide a rotated wave or rotated corrugated sections between the upper frame arm and the lower frame arm which in unique face geometries, maintain flexion against the face, and to reduce or eliminate torsion of the upper frame arm.

It may be an advantage to provide a blended wave or blended corrugated sections between the upper frame arm and the lower frame arm which in certain complex face geometries, reduces the effect of torsion on the arm from having the thick border around the blended wave geometry on the upper frame arm.

It may be an advantage to provide a bevelled hook design for ease of clipping or unclipping in one direction.

It may be an advantage to provide a double bevelled hook design, allowing for easy unclipping when twisting in either clockwise or anticlockwise.

It may be an advantage to provide a bottom headgear strap biasing for improving the angle of drop.

It may be an advantage to provide a single strand tether to reduce interference of tether with the clip interface and to reduce visual complexity of the system.

It may be an advantage to provide perpendicular supports and/or linear supports for increasing the amount of forward biasing of the lower headgear strap further, and to eliminate jaw interference, preventing droppage and twisting of the staps towards the clip-end.

It may be an advantage to use layers of ‘C-shaped’ sheet plastic adhered to the lower headgear strap for reducing drop and twisting of bottom or lower headgear straps.

It may be an advantage to reduce length of bottom strap rigid section to a shorter section, in which the rounded rigid section end can prevent irritation on jaw.

It may be an advantage to provided heat formed linear ridges to previously unsupported bottom or lower headgear strap at the back, while at the front, having the reduced length of bottom strap rigid shorter section. This particular lower headgear strap embodiment provides a significant reduction in twisting of headgear and increasing the height of clip resting place for ease of the user holding and securing to the clip at the lower end of the exoskeleton.

It may be an advantage to provide an updated base panel with a breathable material for improved positioning and comfort and to reduce strap stretch over time.

It may be an advantage to provide structured bottom-straps to get forward biased bottom straps to aid usability and reduce or prevent the clips or lower strap falling behind the ears or behind the head and shoulders.

It may be an advantage to provide Velcro® or hook fastener tabs with “safety bump” to prevent clip slippage without sharp plastic edges to minimise injury for the user.

It may be an advantage to redirect forces to the back of the skull for user comfort.

It may be an advantage to encapsulate the rigid section with a synthetic velour fabric to remove direct contact with a rigid material for user comfort.

It may be an advantage to provide an air delivery tube or elbow that can easily and securely connect to the aperture of the mask. It may also be an advantage to provide a quick release mechanism involving a deformable elastic TPE membrane with memory properties for ease of disconnecting the air delivery tube from the mask aperture.

It may be an advantage to provide an anti-asphyxia valve (AAV) for providing a safety mechanism of the user.

It may be an advantage of providing vent holes to the endoskeleton where it will not be covered by the exoskeleton so the exoskeleton will not hinder the outflow of air from the vent holes.

It may be an advantage of providing the vent hole array to be front facing or angled out to the side depending on ease of the moulding process or steepness in position of the mask.

It may be an advantage to provide the tapered holes in the vent hole array to narrow the air-jet on exit from the mask.

It may be an advantage to provide non-tapered holes to reduce the strength of the air jet and diffuse over a greater region after exit from mask.

It may be an advantage to provide vent holes of variable diameter to allow additional control of air jets.

It may be an advantage to provide vent holes with variable spacing relative to each other such that larger spacing between holes would emit a broader jet-stream of air from the mask, to prevent air jet streams colliding to minimise noise.

It may be an advantage to provide annular vent hole array in the elbow or the air delivery tube as an additional safety mechanism.

It may be an advantage to provide the annular vent hole array to be front-facing or angled or a combination of some holes that are front-facing and some holes that are angled for better air-jet diffusion, which would advantageously disrupt patient less.

It may be an advantage to provide a chamfer angle, hole orientation, and hole taper to allow easy removal of side action mould part, as well as diffusion direction and noise.

It is an object of the present invention to overcome or ameliorate at least one of the disadvantages of the prior art, or to provide a useful alternative.

### Means for Solving the Problem

A first aspect of the present invention may relate to a mask adapted for delivering pressurised air to a user, the mask may comprise an endoskeleton of a first resilient material encapsulated to a lamina of a second resilient material forming a mask body with a cavity, wherein the second resilient material is relatively more flexible than the first resilient material. The lamina may have a face contactable skirt portion extending away from the endoskeleton perimeter, wherein the skirt portion may comprise an outer layer and an inner layer for providing variable contoured thickness to outer lamina segments of the face contactable portion. The face contactable skirt portion may have a flexible cavity edge defining a cavity opening for receiving a nasal region, wherein the endoskeleton and lamina has an aperture, wherein the aperture may be adapted to connect to an air delivery tube. The thinner flexible cavity edge may be adapted to wrap into the cavity, wherein when wrapped, the contoured thicker lamina segments wraps and seals around the sides of the nose. Preferably, the face contactable portion may comprise a nose bridge centre segment, a left and right side of the nose segment, an upper lip segment positioned between a left and right bottom corner segment, and a left and right-side cheek segment.

Preferably, the cavity opening can further receive a mouth region, wherein the thinner flexible cavity edge at the bottom region has a raised edge, wherein the raised edge wraps and seals around the chin and cheek. Preferably, the face contactable portion may comprise a nose bridge centre segment, a left and right side of the nose segment, a chin segment, a left and right bottom corner segment, and a left and right side cheek segment.

Preferably, the endoskeleton has a major segment and at least one minor segments. More preferably, the major segment and the minor segment are of the same material, and wherein the at least one minor segments are hingedly connected to the major segment. Preferably, the left-side nose bridge segment may have a first thickened rib, and wherein the right-side nose bridge segment may have a second thickened rib, wherein the first and second thickened ribs maintains its shape and seal at the nose bridge segment when subjected to pressurised air delivered by the air delivery tube. More preferably, the flexible cavity edge between the left and right-side nose segment via the nasal centre segment may comprise a biased contour connecting between a left-side nose wing and a right-side nose wing, wherein the biased contour allows for an increased seal with the nasal bridge while the nose wings increase surface area allows for reducing air blow out from sides of nose. More preferably, the biased contour is a stress relief radius.

Preferably, the flexible cavity edge may be a polished edge around the edge of the face contacting portion, wherein the polished edge may be greater than 3 mm. Preferably, the left and right-side nose segments may each comprise a contoured segment thicker relative to the nose bridge centre segment. Preferably, the contoured segment of the left and right-side nose segments each have a membrane of a predetermined spiral shape for thickening predetermined portions of the lamina along the nose section. Preferably, the skirt portion may comprise an inner seal lamina adjoining from a first partial section of the left-side nose segment and a second partial section of the right-side nose segment via the nose bridge centre segment. More preferably, the left-side cheek segment and the right-side cheek segment may each comprise stiffening ribs for maintaining the shape of the skirt portion when subjected to pressurised air delivered by the air delivery tube. Preferably, the outer lamina of the face contactable skirt portion and a cantilever inner lamina may each extend from the endoskeleton perimeter at the same location, wherein the cantilever inner lamina has a predetermined thickness. Preferably, the outer lamina may be frosted, and wherein the outer lamina may be made of silicone. Preferably, the mask may further comprise a chin gimble. More preferably, the flexible cavity edge may comprise an extended chin lamina for reducing jaw leak at the chin. Preferably, a top profile of the skirt portion of the chin region may have a ‘V’ shape for allowing the chin region of the skirt portion to contact the chin earlier, when in use. Preferably, the chin region may have a thickness between 0.20 to 0.45 mm thin lamina. More preferably, the chin region may have a 0.35 mm thin lamina for reducing force on chin and to accommodate mouth movement. Preferably, the nasal bridge region has a thickness between 0.20 to 0.45 mm thin lamina for reducing force on centre and/or side of the nasal bridge. More preferably, the nasal bridge region is 0.35 mm thin lamina. Preferably, the endoskeleton may be made from a rigid or semi-rigid or flexible plastic material selected from materials such as polycarbonate, polypropylene, polyester, Acrylonitrile Butadiene Styrene (ABS), thermoplastic elastomer (TPE), polycarbonate-ABS blend, polyethylene polyamide (nylon), or polypropylene, wherein the plastic material of the endoskeleton is relatively rigid compared to the lamina. More preferably, the material has a plastic shore durometer hardness between 40 to 85 Shore D.

Preferably, the mask may further comprise an exoskeleton securable to the encapsulated endoskeleton, the exoskeleton may have an aperture with a shape congruent to the aperture of the encapsulated endoskeleton, wherein when secured, the exoskeleton aperture may be positioned over the encapsulated endoskeleton aperture, such that the air delivery tube may be connectable to the cavity through the exoskeleton aperture and the encapsulated endoskeleton aperture. More preferably, the air delivery tube having a first end and a second end, wherein the first end has a snap connection for sealingly interlocking to the exoskeleton aperture and the encapsulated endoskeleton aperture, and wherein the second end is attachable to a Continuous Positive Airway Pressure (CPAP) apparatus or a bi-level pressure device. More preferably, the first end of the air delivery tube may comprise a snap release mechanism positioned inside the exoskeleton, wherein the mechanism of release may be facilitated by rotating about a fulcrum and deforming an elastic thermoplastic elastomer, which loosens and allows rotation of the first end of the delivery tube relative to the endoskeleton aperture to disengage. More preferably, the air delivery tube may further comprise an anti-asphyxia valve (AAV) positioned in an air inflow channel between the first end and the second end of the air delivery tube. More preferably, the air delivery tube comprises a CO₂ washout channel, which may be an independent channel to the inflow of air from the second end to the first end. Preferably, the endoskeleton may comprise an encapsulated area and a non-encapsulated area, wherein a vent hole array may be positioned at the non-encapsulated area. More preferably, the vent hole array may have a profile that may be at least one selected from the group of: front facing, angled out to the side, tapered, and non-tapered.

Preferably, the encapsulated endoskeleton may have a major segment of the first resilient material, and minor segments of the second resilient and flexible material, wherein the major segment has elongate structural members for providing support to the face contactable skirt portion of at least one segment selected from the group of: the left and right side of the nose segment, the upper lip segment, the left and right bottom corner segment, and the left and right-side cheek segment. More preferably, an exoskeleton is mountable to the front of the endoskeleton; a first frame arm having a first exoskeleton connector securable to a first side of the exoskeleton; a second frame arm having a second exoskeleton connector securable to a second side of the exoskeleton; wherein each of the first frame arm and the second frame arm bifurcates at a middle frame segment forming an upper frame arm and a lower frame arm, wherein the upper frame arm has an upper headgear connector for securing an upper headgear strap, and wherein the lower frame arm has a lower headgear connector for securing a lower headgear strap. Preferably, the first exoskeleton connector and the second exoskeleton connector are each angled between 15° to 25° with respect to the longitudinal axis of the frame arm. Preferably, each of the frame arms may have vertically corrugated portions positioned along the longitudinal axis of the frame arm between the connecting ends. Preferably, a non-corrugated portion may be positioned between the corrugated portion and the upper headgear strap securing means, wherein the non-corrugated portion of a resiliently stiff material may be adapted to reduce twisting of the frame arm. Preferably, the frame arms may be made of a resiliently stiff material. Preferably, the frame arm may have a first corrugated portion and the lower frame arm may have a second corrugated portion, wherein the first corrugated portion may be angled differently to the second corrugated portion. Preferably, the first corrugated portion and the second corrugated portion may be blended.

Preferably, the lower frame arm may have a bevelled hook for receiving and securing a lower headgear clip or the lower headgear clip may have a bevelled hook for receiving and securing the lower frame arm, wherein the bevelled hook may have a bevelled edge. Preferably, the secured lower headgear clip is disengageable from the bevelled hook by pivotally rotating the lower headgear clip out of the hook such that the clip is parallel to a first bevelled edge of the bevelled hook. More preferably, the bevelled hook further comprises a second bevelled edge angled 90° relative to the first bevelled edge, wherein the secured lower headgear clip is disengageable from the bevelled hook by either pivotally rotating the lower headgear clip out of the hook such that the clip is parallel to the first bevelled edge or the second bevelled edge of the bevelled hook. Preferably, the headgear comprises a raised base panel of a resilient flexible material connecting between curved bottom headgear straps for travelling around ears of a user. Preferably, an interior of the curved bottom headgear straps each having a structured polypropylene sheet flanking the raised base panel for providing a relatively rigid structured area, and a soft material encapsulating the structured element of the bottom headgear strap for providing a relatively moderate elasticity to the lower headgear strap. Preferably, an exterior of the upper and lower headgear straps comprises a Velcro® hook fastener tab at the distal end of the straps for preventing clip slippage.

Preferably, the mask may further comprise an elastic tether securable to a lower headgear clip at one end and an elastic loop portion at the other end, wherein the exoskeleton has a hook for receiving and securing the elastic loop portion and the clip.

Preferably, the exoskeleton may have an upper frame portion positioned between the exoskeleton aperture and an upper headgear fastening member, wherein the upper frame portion may comprise a vertical corrugated section partially along the length of the upper frame portion. More preferably, the upper frame portion may further comprise a hinge portion positioned between the exoskeleton aperture and the vertical corrugated section. Preferably, the exoskeleton may have an upper frame portion positioned between the exoskeleton aperture and an upper headgear fastening member, wherein the upper frame portion may comprise a first hinge portion spaced away from a second hinge portion, wherein the hinge portions may be partially along the length of the upper frame portion.

Preferably, the exoskeleton has an upper frame portion positioned between the exoskeleton aperture an upper headgear fastening member, wherein the upper frame portion comprises a corrugated portion partially along the length of the upper frame portion, wherein the profile of the corrugated portion is angled with respect to the vertical axis of the mask. Preferably, the upper frame portion comprises a second corrugated portion spaced apart from the corrugated portion. Preferably, the exoskeleton is corrugated. Preferably, the profile of the corrugated portion is one selected from the group of: sinusoidal, elliptical, triangular, square, and rectangular. More preferably, the corrugated portion has a wall section of a consistent width or more preferably, the corrugated portion has a wall section of varying widths along the length of the exoskeleton.

Preferably, the encapsulated endoskeleton aperture has a non-circular shape profile, wherein the perimeter defining the endoskeleton aperture has one or more frame retention means for interlocking with the back of the exoskeleton. More preferably, the front of the exoskeleton has one or more air delivery tube retention means for interlocking with the air delivery tube. Preferably, the non-circular shape profile is a tear drop shape profile.

Preferably, the mask may further comprise an exoskeleton headgear module mountable to the endoskeleton, wherein the exoskeleton headgear module may comprise a fastening device for connecting the exoskeleton with a headgear, wherein the fastening device may have an elastic memory material connected between an exoskeleton fastening member and a headgear fastening member for securing the fastening members together. Preferably, providing a pulling force to the headgear away from the exoskeleton may disengage the fastening members and stretches the elastic memory material and wherein when the pulling force is released, the elastic memory material may move the fastening members towards each other and self-secures the fastening members together.

Preferably, the exoskeleton headgear module comprises an upper headgear strap and a lower headgear strap, wherein the lower headgear strap comprises a stiffening element such that the lower headgear strap is forward biasing towards the exoskeleton. Preferably, the stiffening element is one chosen from the group of: heat staking resilient materials, a glued resilient material, a stitching of a stiff material, a thicker fabric, a thermoformed shape, a stitching of a hook fastener (rough side) and loop fastener (soft side) to the lower headgear strap such as a Velcro® portion.

In the context of the present invention, the words “comprise”, “comprising” and the like are to be construed in their inclusive, as opposed to their exclusive, sense, that is in the sense of “including, but not limited to”. It may be appreciated that any of the parts and/or modules making up the mask and/or mask assembly, not limited to and including the mask 100, mask 200, endoskeleton 102, exoskeleton 300 for mask 100, bevelled hooks 436, corrugated frame arms, exoskeleton-headgear module with the elastic connectors with memory properties, exoskeleton 400 for mask 200, air delivery tube/elbow 500, headgear 600 with improved base panel and strap arms etc could be used together, each of the these features are individually improvements and that the individual parts and/or modules can be further aspects to the present invention.

The invention is to be interpreted with reference to the at least one of the technical problems described or affiliated with the background art. The present aims to solve or ameliorate at least one of the technical problems and this may result in one or more advantageous effects as defined by this specification and described in detail with reference to the preferred embodiments of the present invention.

### BRIEF DESCRIPTION OF THE FIGURES

FIG. 1 illustrates a back view of a mask showing a face contactable skirt portion of a mask of a preferred embodiment, in which the face contactable skirt portion is marked with a nose bridge centre region, a left-side nose bridge region, a right-side nose bridge region, a chin region, a left-side of nose region, a right-side of nose region, a left-side cheek region, a right-side cheek region, a left-side bottom corner region, and a right-side bottom corner region.

FIG. 2 illustrates a back view of a mask showing a mask with the face contactable skirt portion of a further preferred embodiment having a narrow nose contour joining a left-side nose wing to a right-side nose wing via the nose bridge centre region. This preferred mask embodiment for fitting smaller and flatter noses.

FIG. 3A illustrates a back view of a mask showing a mask with the face contactable skirt portion of a further preferred embodiment having a left-side nose wing not joining to a right-side nose wing as shown and with thickened vertical rib positioned under the skirt portion at the left-side or right side of the nose region.

FIG. 3B illustrates a partial perspective view of the mask of FIG. 3A.

FIG. 4 illustrates a back view of a mask showing a mask with the face contactable skirt portion of a further preferred embodiment having a first thickened vertical rib under the skirt portion at a left-side of the nose region and a second thickened vertical rib under the skirt portion at a right-side of the nose region so as for allowing easy expansion and contraction of the membrane to accommodate wider facial geometries.

FIG. 5 illustrates a back view of a mask showing a mask with the face contactable skirt portion of a further preferred embodiment having a stress relief radius for allowing the skirt portion to expand easier for larger noses.

FIG. 6 illustrates a back view of a mask showing a thin membrane positioned behind a thicker membrane such that the tip reduces pressure from the thick membrane in the event of significant lamina compression.

FIG. 7 illustrates a back view of a mask showing a secondary seal membrane of another preferred embodiment partially covering under the skirt portion from the left-side nose portion and the right-side nose portion, and the secondary seal membrane covering under the skirt portions at the nose bridge side portions and the nose bridge centre portion.

FIG. 8A illustrates a cross-sectional partial region of the mask of FIG. 7 with the skirt portion and the relative orientation to a secondary seal membrane attached to the perimeter of an endoskeleton.

FIG. 8B illustrates the cross-sectional partial region of FIG. 8A engaging with a person's nose bridge showing a person's nose contacting both the skirt portion and the secondary seal membrane.

FIG. 9 illustrates a secondary seal membrane of another preferred embodiment partially covering under the skirt portion from the left-side nose portion, the right-side nose portion, the nose bridge side portions, and the nose bridge centre portion.

FIG. 10A illustrates a cross-sectional partial region of the mask of FIG. 9 with the skirt portion and the relative orientation to a secondary seal membrane attached to the perimeter of an endoskeleton.

FIG. 10B illustrates the cross-sectional partial region of FIG. 10A engaging with a person's nose bridge showing a person's nose contacting the skirt portion only.

FIG. 11A illustrates a thickening/stiffening rib for positioning under the skirt portion.

FIG. 11B illustrates the thickening/stiffening rib positioned under the skirt portion.

FIG. 12 illustrates a back view of a mask with a thick polished edge around the edge of the skirt portion for an improved seal around the edge in another preferred embodiment.

FIG. 13 illustrates a back view of a mask with specific polished areas of the membrane which does not cover the entire membrane so as to improve seal contact without fully sacrificing comfort in another preferred embodiment.

FIG. 14 illustrates a back view of a mask with a small localised dual wall at one side of the nose and another small localised dual wall at the other side of the nose in which the dual localised only at the sides of the nose to improve seal around the nose. In this contoured localised dual wall embodiment, cheek compression does not affect the mask's performance.

FIG. 15A illustrates a back view of a mask with a contoured thickened layer positioned at a skirt portion of the left-side and the right-side of the nose region.

FIG. 15B illustrates a partial back view of a mask with a translucent skirt portion with the contoured thickened translucent layer supported by the schematic drawing of FIG. 15A, in which schematic drawing of FIG. 15A showing the boundaries and shape of the thickened layer.

FIG. 16 illustrates a back view of a mask with a thin membrane layer at the edge of the skirt portion at the cheek regions, the bottom corner regions, and the chin region in another preferred embodiment. As stiffness is not required right at the edges as shown in the indicated shaded regions, so the membrane layer can be thinned earlier to resolve mouth leak.

FIG. 17A illustrates a thickening/stiffening rib for positioning under the skirt portion for tuning stiffness in compression.

FIG. 17B illustrates the thickening/stiffening rib positioned under the skirt portion such that it reduces blow out and cheek leak.

FIG. 18 illustrates a partial side profile of a curled membrane or wrapped skirt portion joined to the buffer membrane portion for improving the likelihood of the skirt portion rolling.

FIG. 19 illustrates a partial side profile of a folded skirt portion joined to the buffer membrane portion, in which folded profiles are more likely to bend or crush.

FIG. 20 illustrates a back view of a mask with the positioning of the thicker membrane layer and the thinner membrane layer of the skirt portion as well as dual wall membranes positioned at the left-side and right-side of the bottom region in another preferred embodiment. The localised dual wall membrane includes an inner membrane which provides stiffness for the foundation of the mask. And the thickness and profile can be tuned to optimise stiffness required. Length of the dual wall can be adjusted to seat in the most comfortable place on the cheek.

FIG. 21 illustrates a partial side profile of an inner membrane of a preferred embodiment, which can be a simple cantilever beam which may not necessarily be a contoured membrane.

FIG. 22 illustrates a contextual view of the inner membrane as a simple cantilever beam of FIG. 21 with respect to the mask.

FIG. 23 illustrates a partial side profile of another preferred embodiment, in which the skirt portion has a thin outer membrane for an improved seal and a variable thickness inner membrane. The variable thickness inner wall combines the membrane performance of the current variable wall thickness cushion membranes/skirt portion with the added compliance and seal benefit of a thin seal outer wall. The outer wall could also be optimised for comfort in a further preferred embodiment.

FIG. 24 illustrates a back view of the mask having a simple multi segment dual wall of another preferred embodiment. The multiple dual walls within the same cushion can decouple the stiffness in different areas of the cushion allowing for a more personalised fit especially for the cheek regions. The individual membrane thickness, lengths and profiles can be tuned.

FIG. 25 illustrates a back view of the mask having a complex multi segment dual wall of another preferred embodiment. The more segments can increase the ability for the cushion to adapt to unique face shapes especially for the cheek regions.

FIG. 26 illustrates a schematic of the chin region of the skirt portion engaging with the chin or lower lip region of the face. The mask having a chin gimble of a preferred embodiment for increased compliance for jaw drop to reduce jaw leak.

FIG. 27 illustrates a schematic of the chin region of the skirt portion engaging with the chin or lower lip region of the face. The mask having an extend chin membrane of a preferred embodiment for increased compliance for jaw drop to reduce jaw leak.

FIG. 28 illustrates a back view of the mask having showing the extended chin membrane of the preferred embodiment as shown in FIG. 27.

FIG. 29 illustrates a back view of the mask of another preferred embodiment showing the cushion concept or skirt portion concept of a single wall. In this embodiment, the skirt portion has thickened wall sections to pinch sides of nose in which the thick sections provide stiffness where required, a thin and highly compliant nose bridge section, wings on the side of the nose to reduce air blow out, thin inner membrane at the cheek sections (either extended in or thinned early) to reduce mouth leak, thin and highly compliant chin membrane sections to reduce mouth leak, and frosted everywhere for comfort.

FIG. 30 illustrates a back view of the mask of another preferred embodiment showing another cushion concept or skirt portion concept of a multi segment dual wall. In this embodiment, the skirt portion has thin and highly compliant nose bridge, multi dual wall membranes segments optimized for comfort, variable thickness inner wall, thin and highly compliant chin section, and frosted outer membrane for comfort.

FIG. 31 illustrates a partial side profile of a curled membrane or wrapped skirt portion joined to the endoskeleton perimeter for improving the likelihood of the skirt portion rolling, showing an inner membrane with variable thickness.

FIG. 32 illustrates a back view of the mask of another preferred embodiment showing the framework inside the cavity made from rigid plastic such as polycarbonate or nylon. Due to the rigidity, the mask framework transfers force from the frame to the cushion membrane or skirt portion and so transfer force to the edge of the cushion membrane equally. The rigid plastic of the endoskeleton applies pressure to the cushion membrane edge.

FIG. 33 illustrates a back view of the mask of another preferred embodiment with varying stiffness of the cushion membrane being stiffer at the cheeks and the side of the nose and very flexible at the nose bridge and chin. As the force applied by the cushion varies around the membrane, the force required by the endoskeleton will also vary and hence the stiffness of the chassis can vary without impacting cushion performance.

FIG. 34 illustrates a back view of the mask of another preferred embodiment showing endoskeleton stiffness regions, in which the stiffer regions align with stiff cushion regions.

FIG. 35 illustrates a front view of the mask of another preferred embodiment showing a variant of the endoskeleton shape.

FIG. 36 illustrates a front perspective view of a mask of another preferred embodiment showing another variant of the endoskeleton shape, the mask covering the person's nose and mouth regions, more suitable for persons with a wider mouth.

FIG. 37 illustrates a front view of the mask of another preferred embodiment showing another variant of the endoskeleton shape, in which this variant is suitable for a narrow nose, in which the arrows illustrate the direction that the endoskeleton can flex.

FIG. 38A illustrates a front view of the mask of another preferred embodiment showing another variant of the endoskeleton shape, in which the arrows illustrate the direction that the endoskeleton can flex.

FIG. 38B illustrates a partial side view of FIG. 38A.

FIG. 38C illustrates a partial side schematic view of the endoskeleton of FIG. 38A is mounted to the cushion or skirt portion of the mask.

FIG. 39A illustrates a side view of the mask module of another preferred embodiment showing the endoskeleton encapsulated by a flexible layer and the skirt portion, the perimeter of the endoskeleton shown.

FIG. 39B illustrates a top view of the mask module of FIG. 39A from the nose bridge centre towards the chin region, showing the polished edge and contours of the mask.

FIG. 40A illustrates a front view of a mask of another preferred embodiment showing the dual membrane at spiral.

FIG. 40B illustrates a cross-section view (A-A) of FIG. 40A showing that the endoskeleton providing the support to the membrane, in which the membrane is angled to direct force normal to the nose.

FIG. 41 illustrates a side view of the mask of another preferred embodiment showing the endoskeleton encapsulated by a flexible layer and the skirt portion, the perimeter of the endoskeleton can be changed as drawn overlayed. And an indicative portion overlayed showing possible variations by possible removal of the indicated endoskeleton section as drawn.

FIGS. 42A to 42E are schematic front views of a mask with an endoskeleton encapsulated by a flexible membrane according to five different preferred embodiments of the present invention.

FIG. 43 illustrates a top view of a mask with an endoskeleton encapsulated by a flexible membrane in which a frame (drawn overlayed) can be used as an exoskeleton to support the cushion.

FIG. 44 illustrates a back view of a mask with an endoskeleton encapsulated by a flexible membrane according to a different preferred embodiment of the present invention, in which the mask may be a pear or avocado shape. The thin membrane may seal below the sellion and the narrow cushion opening for sealing against the narrow noses. When the cushion rolls in, when in use, the surface is flatter to seal against the skin. The polished edge may interfere with widest nose width but flexible enough to curl or wrap away. This shape fits majority of mouth widths.

FIG. 45 illustrates the relative flatness of the membrane in the natural state for the mask of FIG. 44; and FIG. 46 illustrates that when the membrane rolls, more of a gap appears for the pear-shaped mask. The endoskeleton having a major segment and a minor segment of the same resilient material, the major segment having a hinge-like configuration to the minor segment. This variant of the endoskeleton allows for increasing stiffness and support at the nose region.

FIG. 47 illustrates a top view of the mask, the skirt portion may be orientated in a ‘V’ shape to allow the membrane to contact the chin earlier when putting on. As it is a ‘V’ shape, there is sufficient height to not put undue pressure on the chin, and contact chin earlier and allow for mouth movement. The skirt portion may be of a sufficient depth to allow cushioning to the face. The mask may allow headgear load to transfer vertically into the side of the mouth, thereby reducing horizontal component of headgear force.

FIG. 48 illustrates a schematic view of an outer membrane biased so that it will readily contact the skin of the user.

FIG. 49 illustrates a schematic view of the skirt portion or cushion module, wherein mask of the preferred embodiment has a 0.4 mm thin area to conform to different sizes of nose bridge, a thin ‘flap’ to conform to various nose geometries and allowing air pressure to push the ‘flap’ against the nose; gradual transition between thickness areas so that membrane does not fold, local thickness to direct force normal to the side of the nose (hug nose); spiral shape to travel along the nose and flex away if force is high; mouth corner moved lower compared to naturally line up with mouth better; 0.4 mm thin area to reduce force on chin and accommodate mouth movement.

FIG. 50 illustrates a side view of a mask of another preferred embodiment having part lines to be smoother for reducing change in curvature, in which the force deflection curve to be similar across thick regions.

FIG. 51A illustrates a side view of a mask of another preferred embodiment, the mask having an endoskeleton for preventing cushion or skirt portion from blowing out and help keep cushion in position; the endoskeleton having a major section and a minor segment from the space left behind by the cutaway portions. The endoskeleton cutaway may allow the cushion to flex up and/or out to accommodate different sized cheeks. The endoskeleton proximal to the aperture can direct load onto the cushion. The wider region to anchor cushion to side of user's mouth. There is also shown thin areas to allow the cushion assembly to flex and the endoskeleton can limit the amount of cushion movement up and out. There is also shown silicone bands being used for acting as a tensioner to transfer force in alternative directions. There may be a nub which centres the endoskeleton within cushion wall thickness.

FIG. 51B illustrates a partial back view of the mask of FIG. 51A.

FIG. 51C illustrates a partial back perspective view of the mask of FIG. 51A.

FIG. 52 illustrates a partial perspective view of a framework or an exoskeleton of a preferred embodiment for mounting to an endoskeleton of any of the previously described embodiments. In this embodiment, this framework has a first hinge portion and a second hinge portion positioned along a left/right-side of the framework. The first hinge portion and the second hinge portion are spaced away relatively from each other. And the hinge portions are positioned between the central aperture of where the tube is connected and the headgear mounting means.

FIG. 53 illustrates a partial perspective view of another framework of another preferred embodiment similar to the framework of FIG. 52, in which the second hinge portion is replaced by a corrugated portion along a short predetermined length along the framework arm. The corrugated portion having a first predetermined wavelength. The first hinge portion and the corrugated portion are spaced away relatively from each other. And the corrugated portion positioned between the first hinge portion and the headgear mounting means at the end of the framework. This particular corrugation gives better hugging where the face curvature changes the most.

FIG. 54 illustrates a partial perspective view of another framework of another preferred embodiment similar to the framework of FIG. 53, in which the corrugated portion is also along a short predetermined length along the framework arm. The corrugated portion having a second predetermined wavelength. The second predetermined wavelength is relatively longer than the first predetermined wavelength of the framework as depicted in FIG. 53. The first hinge portion and the corrugated portion are spaced away relatively from each other. And the corrugated portion positioned between the first hinge portion and the headgear mounting means at the end of the framework. This bigger corrugation pitch along a longer distance and it provides a more gradual bending, better force transfer and perform less like a hinge.

FIG. 55 illustrates a partial perspective view of another framework of another preferred embodiment, in which this embodiment has a corrugated portion positioned between the central aperture and the headgear mounting means at the end. This framework advantageously allows to contact the face earlier as the longer corrugation and bigger pitch hugs the face better, and has less external force to break seal in dynamic movements.

FIG. 56 illustrates the framework of FIG. 55 in use with a person's face.

FIG. 57 illustrates the framework having to flex at least 13 mm horizontally on each side.

FIG. 58 illustrates a back view of the mask of another preferred embodiment with the framework attached over the endoskeleton. The framework having an upper portion integrally connected to a lower portion. The upper portion having vertically corrugated portion along the longitudinal length. This particular embodiment having a ‘T’/‘I’-shape.

FIG. 59A and FIG. 59B illustrate alternative shapes being ‘X’-shape and a single bar.

FIG. 60 illustrates a section R-R side view of the preferred embodiment shown in FIG. 58.

FIG. 61A illustrates a section A-A side view of the preferred embodiment shown in FIG. 58.

FIG. 61B illustrates a section B-B side view of the preferred embodiment shown in FIG. 58.

FIG. 61C illustrates a section C-C side view of the preferred embodiment shown in FIG. 58.

FIGS. 62A to 62G illustrates a person putting on and securing a mask with a headgear attached. The securing means at the lower portion of the framework may have elastic members in connection to the left and right lower portion of the headgear allowing a person to wear. As shown in FIG. 62F, there is a tightening mechanism to secure this mask and headgear embodiment when comfortably positioned.

FIGS. 63A to 63D illustrates an air delivery tube of a preferred embodiment, in which the air delivery tube has rounded buttons at the side to activate release mechanism of the delivery tube when connected to the central aperture of the mask. An elastic TPE membrane which surrounds buttons in which the geometry of this regions creates a fulcrum about which the release mechanism can activate. In this embodiment, there is also a snap release mechanism that sits inside the frame. There is also shown an AAV located on the front of bend of elbow.

FIG. 63E illustrates vent holes in the cushion of a mask of another preferred embodiment, in which the vent holes are angled out to the side.

FIG. 63F illustrates a side view of the desired vent direction.

FIG. 63G illustrates a vent hole array in area of endoskeleton without silicone.

FIG. 63H illustrates a cross-sectional view of the vent hole and the endoskeleton without silicone.

FIG. 63I illustrates a framework mounted to the endoskeleton with the vent holes of the endoskeleton as shown in embodiment of FIG. 63G.

FIGS. 64A and 64B illustrates an annular vent hole array in the air delivery tube or elbow in another preferred embodiment, in which the vent holes are front facing and/or angled for better diffusion.

FIG. 65A illustrates a partial back view of the elbow of a non-tapered vent hole.

FIG. 65B illustrates a cross-sectional side view of the elbow with a non-tapered vent hole.

FIG. 66A illustrates a partial back view of the elbow of a tapered vent hole.

FIG. 66B illustrates a cross-sectional side view of an elbow with a tapered vent hole and an anti-asphyxia valve (AAV) across the airflow channel

FIG. 66C illustrates a cross-sectional side view of the elbow of FIG. 66B with a tapered vent hole and the anti-asphyxia valve (AAV) moved to the corner of the elbow such that the airflow channel is unobstructed.

FIG. 67 illustrates an elbow-frame-cushion interface of a preferred embodiment, in which the lip seal is overmoulded onto cushion or endoskeleton.

FIG. 68A illustrates an assembled a mask assembly of another preferred embodiment.

FIG. 68B illustrates the disassembled mask assembly of FIG. 68A, showing the frame, endoskeleton cushion module, and the headgear.

FIG. 69 illustrates a front perspective view of the endoskeleton cushion module of FIG. 68A, which covers the nose region.

FIG. 70 illustrates a perspective view of the frame of FIG. 68A.

FIG. 71A illustrates a back perspective view of the endoskeleton cushion module of FIG. 68A, which covers the nose region.

FIG. 71B illustrates a focused view of the endoskeleton cushion module of FIG. 71A, showing the raised edge for an improved stretched at the lower nose region.

FIG. 72 illustrates a perspective view of the headgear.

FIG. 73A illustrates an endoskeleton cushion module of another preferred embodiment and FIGS. 73B to 73E illustrates the endoskeleton used as shown in FIG. 73A in different perspective views.

FIGS. 74 to 79 illustrates endoskeleton shapes of different preferred embodiments. More particularly, taking the endoskeleton shown in FIG. 74 as a reference, the endoskeleton shown in FIG. 75 is a full cup version; the endoskeleton shown in FIG. 76 has a relatively wider wing compared to the endoskeleton shown in FIG. 74; the endoskeleton shown in FIG. 77 has a relatively slimmer wing compared to the endoskeleton shown in FIG. 74; the endoskeleton shown in FIG. 78 has a relatively wider elbow bore ring compared to the endoskeleton shown in FIG. 74; the endoskeleton shown in FIG. 79 has a relatively shorter wing compared to the endoskeleton shown in FIG. 74.

FIG. 80 illustrates the endoskeleton of FIG. 74 with a slot through each wing, in which the slot allows for a mechanical interlock with a frame.

FIGS. 81 to 83 illustrates a perspective view of an endoskeleton cushion module of different preferred embodiments.

FIG. 84 illustrates a perspective view of a variety of different endoskeletons and endoskeleton cushion modules.

FIG. 85 illustrates a person using an assembled mask assembly of a preferred embodiment.

FIG. 86 illustrates another perspective view of a person wearing the assembled mask assembly.

FIG. 87 illustrates a back view of the frame showing the addition of ribs and the positioning of the snaps for mounting to the endoskeleton cushion module.

FIG. 88 illustrates a side view of a frame arm of a preferred embodiment for use with an endoskeleton cushion module.

FIG. 89 illustrates a top view of both frame arms attached to the central portion of the frame.

FIG. 90 illustrates the positioning of the frame arm alongside a side of a face of the user.

FIG. 91 illustrates a person using the mask assembly of a preferred embodiment showing the flexibility of the frame arm.

FIG. 92A illustrates a bifurcated frame arm of another preferred embodiment having a vertically corrugated portion at the bifurcated upper portion and the lower portion.

FIG. 92B illustrates a bifurcated frame arm of another preferred embodiment having no wave.

FIG. 92C illustrates a bifurcated frame arm of another preferred embodiment having a rotated wave

FIG. 92D illustrates a bifurcated frame arm of another preferred embodiment having a blended wave.

FIGS. 93A to 93C illustrates a hook from the endoskeleton cushion module hooked to a clip showing the unclipping motion.

FIG. 94A illustrates a hook of another preferred embodiment engaged with a clip in the shown configuration.

FIG. 94B illustrates the clip disengaging with the hook of FIG. 94A when angled or rotated by 45°.

FIG. 95 illustrates a top view of the hook engaged with the clip in FIG. 94A.

FIG. 96 illustrates a top view of the clip disengaging with the hook of FIG. 94B.

FIG. 97 illustrates a mask assembly using a frame arm of a rotated wave.

FIG. 98A to 100B illustrate different face shapes used for testing with various mask assemblies using a default endoskeleton cushion module.

FIG. 101A to FIG. 103B illustrate different face shapes used for testing with various mask assemblies using a ‘variant 4’ endoskeleton shape referred in the group of endoskeleton configurations shown in FIG. 84.

FIG. 104A to FIG. 106B illustrate different face shapes used for testing with various mask assemblies using a ‘variant 5’ endoskeleton shape referred in the group of endoskeleton configurations shown in FIG. 84.

FIG. 107A illustrates a front view of a tall and narrow face of a person wearing a mask assembly with a wave geometry in the top arm of the frame and bent at the cheek and at the terminal; and FIG. 107B illustrates a side view of FIG. 107A.

FIG. 108A illustrates a side view of a normal face of a person wearing a mask assembly with a wave geometry in the top arm of the frame and bent at the cheek and at the terminal; and FIG. 108B illustrates a front view of FIG. 108A.

FIG. 109A illustrates a front view of a short and wide face of a person wearing a mask assembly with a wave geometry in the top arm of the frame and bent at the cheek and at the terminal; and FIG. 109B illustrates a side view of FIG. 109A.

FIG. 110A illustrates a side view of a tall and narrow face of a person wearing a mask assembly with a wave geometry in the top arm of the frame and bent at the cheek and at the terminal; and FIG. 110B illustrates a front view of FIG. 110A.

FIG. 111A illustrates a side view of a normal face of a person wearing a mask assembly with a wave geometry in the top arm of the frame and bent at the cheek and at the terminal; and FIG. 111B illustrates a front view of FIG. 111A.

FIG. 112A illustrates a side view of a short and wide face of a person wearing a mask assembly with a wave geometry in the top arm of the frame and bent at the cheek and at the terminal; and FIG. 111B illustrates a front view of FIG. 111A.

FIG. 112A illustrates a front view of a short and wide face of a person wearing a mask assembly with a wave geometry in the top arm of the frame and bent at the cheek and at the terminal; and FIG. 112B illustrates a side view of FIG. 112A.

FIG. 113A illustrates an upper part of the headgear strap attached to the sides of the upper frame to an endoskeleton cushion module that covers the nose and the mouth regions. This particular embodiment having a left loop portion and a right loop portion near the bottom corners of the endoskeleton cushion module. When the lower parts of the headgear strap are not secured to the respective loop portion, and when held, it shows the straps being easily tangled and not user friendly.

FIG. 113B illustrates an upper part of the headgear strap attached to the sides of the upper frame to an endoskeleton cushion module that covers the nose and the mouth regions. This particular embodiment having a left magnetic portion and a right magnetic portion near the bottom corners of the endoskeleton cushion module. When the lower parts of the headgear strap are not secured to the respective magnetic portion, and when held, it shows that the magnets easily attach to each other and is also not user friendly.

FIGS. 113C and 113D illustrate different perspective views of the attached upper frame and the lower frame via magnetic means from the embodiment as shown in FIG. 113B.

FIGS. 114A to 114C illustrates an elastic tether securable to a left loop portion or a right loop portion near the bottom corners of an endoskeleton cushion module embodiment. The tether for reducing the interference between the clip and interface of the frame.

FIG. 115A illustrates a front perspective view of the elastic tether tethered to a loop portion of the bottom corner of the endoskeleton cushion module embodiment that covers the nose and mouth regions.

FIGS. 115B and 115C illustrates a right-side view and left-side view of the elastic tether tethered to a loop portion of the bottom corner of the endoskeleton cushion module embodiment that covers the nose region only.

FIG. 116 illustrates a bottom strap of a preferred embodiment having perpendicular supports along the longitudinal axis. The perpendicular supports having a predetermined spacing from each other.

FIG. 117 illustrates another bottom strap of another preferred embodiment having perpendicular supports and linear supports along the longitudinal axis. The perpendicular supports having a predetermined spacing from each other; and the linear supports also having a predetermined spacing from each other.

FIGS. 118A and 118B illustrates the twisting direction of the bottom strap of the preferred embodiment shown in FIG. 116.

FIG. 118C illustrates the twisting direction of the bottom strap of the preferred embodiment shown in FIG. 117.

FIG. 119 illustrates a zoomed in view of the headgear with a bottom strap having a magnet at the securing end.

FIG. 120 illustrates a headgear secured to a mask assembly of another preferred embodiment, in which the headgear uses the magnetic securing end at the bottom strap of the headgear.

FIGS. 121 and 122 illustrates the bottom strap disengaged from the magnetic clip interface at the bottom corners of the mask assembly of FIG. 120.

FIG. 123 illustrates a reduced length of relatively harder section to improve comfort and ease of tab adjustment without jeopardizing forward biasing.

FIGS. 124 and 125 illustrates the bottom strap disengaged from the magnetic clip interface at the bottom corners of the mask assembly of FIG. 120.

FIG. 126 illustrates a partial front view of the headgear of FIG. 123.

FIG. 127 illustrates a partial back view of the headgear of FIG. 126, showing the use of a bottom strap of another preferred embodiment having perpendicular supports and linear supports along the longitudinal axis. The perpendicular supports having a predetermined spacing from each other; and the three linear supports also having a predetermined spacing from each other.

FIGS. 128 to 130, and FIGS. 132 and 133 illustrates the bottom strap disengaged from the magnetic clip interface at the bottom corners of the mask assembly of FIG. 120.

FIG. 131 illustrates a partial zoomed in view of a part of the headgear.

FIG. 134 illustrates a headgear of a preferred embodiment showing the improved base panel and attachments.

FIG. 135 illustrates an overlayed image of other headgears compared to the improved headgear preferred embodiment for use with the mask assembly.

FIG. 136 illustrates a person wearing a mask with a structured headgear that has forward biased bottom straps which are shown to be not attached to the bottom securing means for showing how the bottom strap reliably brought forwards of the person's shoulders. The forward biased bottom straps allow for easy access for a person with limited dexterity that has difficulty reaching behind their shoulders, but equally beneficial for any wearer as it is more convenient to use when the lower headgear straps do not fall behind the head or shoulders, leading to the lower headgear straps being grasped.

FIG. 137 illustrates a partial side of the structured headgear showing the top strap and a bottom strap of the same side, in which the curved bottom strap guides the forces clear of the ear and off the neck, whilst the forward biasing prevents the headgear strap and/or the headgear clips (not shown in this figure) from falling behind the shoulder.

FIG. 138A illustrates the force vectors of a person wearing the mask with a forehead support but without a frame supporting the upper portion of the mask. While the top of the head will be comfortable in this configuration, as the force vector crosses onto the wearer's neck, it will cause discomfort to the wearer (indicated in dashed lines).

FIG. 138B illustrates the force vectors of a person wearing the mask with a frame supporting the upper portion of the mask only. While the top of the head will be comfortable in this configuration, as the force vector crosses onto the wearer's neck, it will cause discomfort to the wearer (indicated in dot dashed lines)

FIG. 138C illustrates the force vectors of a person wearing the mask with a frame supporting the upper portion of the mask as well as a structured bottom strap supporting the lower/bottom portion of the mask. As this configuration shifts the force vectors beyond the wearer's neck, the force vectors at the upper part of the head are comfortable as well as the lower part of the head behind the end.

FIGS. 139A and 139B shows the strap tension of the headgear straps that may cause discomfort as the head moves. This tension is applied to the back of the neck. There is less required strap extension when the point of distance is originating from the back of the skull.

FIG. 140 illustrates the exterior view of a headgear showing the custom crown strap, upper strap and the bottom straps, and the custom base panel.

FIG. 141 illustrates the interior view of the headgear of FIG. 140, showing the which parts of the headgear having no/very little elasticity, moderate elasticity, and rigid modification.

FIG. 142 illustrates the interior view of the headgear of FIG. 140, showing the polypropylene sheet to provide structured bottom strap support, a zig zag stitch adjoining the base panel to the left and the right sides, a heat stamped bottom strap, and showing the soft material for encapsulation of structured elements so that a wearer's skin will be in contact to the soft material.

FIG. 143 illustrates an overview of the headgear design showing the careful consideration of the usability of the wearer.

FIG. 144A illustrates another embodiment of the headgear design with rigid sections, which may be sheets of polypropylene, which may be positioned at the upper portion of the bottom strap. The sheets of polypropylene are hidden as it may be encapsulated under synthetic velour fabric.

FIG. 144B illustrates a back view of another embodiment of the headgear design without rigid sections positioned at the upper portion of the bottom strap, in which the rigid sections removed may offer more comfort to the wearer.

FIG. 144C illustrates a front view of the embodiment of the headgear design of FIG. 144B.

FIG. 145A illustrates a front view of a frame mountable to an endoskeleton-cushion module of another preferred embodiment of the invention. In this embodiment, the endoskeleton can be able to snap and secure to the back of the frame.

FIG. 145B illustrates a perspective view of the embodiment as shown in FIG. 145A.

FIG. 145C illustrates a partially assembled view of the mask showing a non-circular shape or a tear drop shape, which is not assembled about the elbow bore, which ensures alignment of the frame to the endoskeleton-cushion module. As shown, there may be at least one frame retention snaps spaced substantially equidistantly around the perimeter of the non-circular shape.

FIG. 145D illustrates a cross-sectional schematic view of the endoskeleton-cushion module with the frame and the elbow attached, wherein the cross-section shows how the endoskeleton-cushion module can snap or interlock to the back of the frame, and also showing how the front of the frame can also snap or interlock with an elbow part.

FIG. 145E illustrates a zoomed-in portion of FIG. 145D showing the interlocking aspects of the different parts in this preferred embodiment.

FIG. 146A illustrates a front view of a frame mountable to a endoskeleton-cushion module of an another preferred embodiment of the invention. In this embodiment, the elbow may interlock with the endoskeleton-cushion module rather than interlocking with the frame (as shown in FIGS. 145D and 145E). In this preferred embodiment, the endoskeleton may have a frame securing means at a first location, and an elbow securing means at a second location.

FIG. 146B illustrates a perspective view of the embodiment as shown in FIG. 146A.

FIG. 146C illustrates a partially assembled view of the preferred embodiment as shown in FIG. 146A, showing the tear drop shape or the non-circular shape for ensuring alignment of the frame or exoskeleton to the endoskeleton-cushion module.

FIG. 146D illustrates a cross-sectional schematic view of the endoskeleton-cushion module with the frame and the elbow attached, wherein the cross-section shows how the endoskeleton-cushion module can snap or interlock to the frame at the first location, and wherein the endoskeleton-cushion module can snap or interlock to the elbow part at a second location.

FIG. 146E illustrates a zoomed-portion of FIG. 146D without showing the connection of the elbow part but showing the elbow retention cavity to interlock with the elbow part. This figure shows how the endoskeleton-cushion module is mounted to the back of the frame.

FIG. 147 illustrates a perspective view of a frame or an exoskeleton mounted to an endoskeleton-cushion module of a preferred embodiment.

FIG. 148 illustrates a perspective view of a frame or an exoskeleton mounted to an endoskeleton-cushion module of another preferred embodiment, in which this embodiment has a corrugated frame portion positioned along an upper arm of the frame.

FIG. 149 illustrates a zoomed-in view of the corrugated portion of FIG. 148.

FIG. 150 illustrates a perspective view of a frame or an exoskeleton mounted to an endoskeleton-cushion module of another preferred embodiment, in which this embodiment has a corrugated frame portion positioned along the centre portion and a partial corrugated frame portion positioned along a lower arm of the frame.

FIG. 151 illustrates a perspective view of a frame or an exoskeleton mounted to an endoskeleton-cushion module of another preferred embodiment, in which this embodiment, the entire frame is corrugated.

FIG. 152 illustrates a schematic view of a sinusoidal or circular profile corrugation for use of a corrugated frame portion.

FIG. 153 illustrates a schematic view of an elliptical profile corrugation for use of a corrugated frame portion.

FIG. 154 illustrates a schematic view of a triangular profile corrugation for use of a corrugated frame portion.

FIG. 155 illustrates a schematic view of a square or rectangular profile corrugation for use of a corrugated frame portion.

FIG. 156 illustrates a schematic representative corrugation where there is consistent wall section, and that the corrugated portion is made from a resilient material allowing elongation under headgear tension.

FIG. 157 illustrates a side view of another preferred embodiment of the frame mounted to the endoskeleton-cushion module, in which the corrugations may be diverted at an angle θ° relative to the vertical axis of the mask.

FIG. 158 illustrates a side view of another preferred embodiment of the frame mounted to the endoskeleton-cushion module, in which the corrugations are parallel to the vertical axis of the mask.

FIG. 159 illustrates a side view of another preferred embodiment of the frame mounted to the endoskeleton-cushion module, in which the frame arm may have multi-segment corrugations (one or more segments), where the multi-segment corrugations may comprise a first corrugated portion/segment spaced apart from a second corrugated portion/segment. As shown in this figure, the corrugations may be diverted at an angle θ° relative to the vertical axis of the mask.

FIG. 160 illustrates a side view of another preferred embodiment of the frame mounted to the endoskeleton-cushion module, in which the frame arm may have a corrugation portion/segment positioned proximal of the centre of the frame compared to the positioning as shown in the embodiment in FIG. 157.

FIG. 161 illustrates a side view of a wearer wearing a frame mounted to an endoskeleton-cushion module with a headgear with unbiased lower headgear straps. The headgear's upper strap secured to the strap receiving means located at the end of the upper arm of the frame. This figure shows that the unbiased lower headgear straps falls behind the neck of the wearer and that the hook for securing to the lower portion of the mask is relatively far away from each other.

FIG. 162 illustrates a side view of a wearer wearing a frame mounted to an endoskeleton-cushion module with a headgear with a forwarding biasing lower headgear straps. The headgear's upper strap secured to the strap receiving means located at the end of the upper arm of the frame. This figure shows that the forward biasing lower headgear straps falls forward or in front of the wearer's shoulder showing that the hook for securing to the lower portion of the mask is relatively closer to each other.

FIG. 163 illustrates a targeted view of a resilient material and its relative positioning to bias a lower headgear strap forward.

FIG. 164 illustrates a targeted view of stitched seams stitched to strap to stiffen the lower headgear strap.

FIG. 165 illustrates a targeted view of a Velcro® or a hook and loop accessory that could be retrofitted to the back/external surface of the lower headgear strap to provide more stiffness and to add forward bias.

FIG. 166 illustrates a perspective view of the endoskeleton-cushion module of another preferred embodiment, showing the cushion rib at nose bridge contoured inwards to improve comfort.

FIG. 167 illustrates another perspective view of the endoskeleton-cushion module of FIG. 166 showing the spiral thickened side of the nose section to optimise load distribution and compliance along nose contour for certain wearer face shapes. In this embodiment, there is a increase in nose bridge depth.

FIG. 168 illustrates a back view of the endoskeleton-cushion module of FIG. 167 showing that the thicker parts of silicone spiral highlighted allows transfer cushion force around side and contour of the nose. It is advantageous for parts of nose that easily changes geometry quite rapidly.

FIG. 169 illustrates a front view of a wearer wearing the endoskeleton-cushion module of FIG. 168 showing a nice and snug fit to the face.

FIG. 170 illustrates a perspective view of an exoskeleton or a frame of a preferred embodiment showing that the center of the frame is suitably shaped so as to transfer force to the endoskeleton via contact points on the endoskeleton. In this preferred embodiment, the frame may have a retention snap to interlock with the endoskeleton-cushion module. There is also shown that the lower frame portion may have bumps to improve ease of removal of a hook tethered to the lower headgear strap.

FIG. 171 illustrates a front view of a wearer wearing the frame of FIG. 170 that is secured to the front of the endoskeleton-cushion module of FIG. 169.

FIG. 172 illustrates a side view of FIG. 171.

FIG. 173 illustrates a side view of the just mask of FIG. 172 showing that the elbow bore can align to the frame attached to the endoskeleton-cushion module. The upper part of the frame can advantageously transfer force to the endoskeleton-cushion module via contact points on the endoskeleton.

FIG. 174 illustrates the schematic consideration of the cushion curvature, thickness, angle and length required at the bottom corners of the lip for the mask where the mask must not leak both when the wearer's stationary and with foreseeable dynamic movements.

FIGS. 175 and 176 illustrates a zoomed-in view of the bubble nose bridge having a spiral thickened side of the nose section to optimise load distribution and compliance along nose contour for certain wearer face shapes, and improving comfort.

FIG. 177 illustrates a perspective view of another endoskeleton-cushion module have a whisker endoskeleton design, which is observed to have seal stability.

FIG. 178 illustrates a zoomed-in view of a bottom corner of the cushion where the contour is smoothly curved.

FIG. 179 illustrates a mask with eroteme matching benchmark mask.

FIGS. 180 and 181 illustrates partial view of a smoothed and blended spiral of an endoskeleton-cushion module, showing the blended spiral having a predetermined thickness and its preferred geometrical positioning along the side of the nose for both nose sides.

FIG. 182 illustrates a side view of a wearer of the endoskeleton-cushion module of FIG. 180 or 181.

FIG. 183 illustrates a front view of the wearer of the endoskeleton-cushion module of FIG. 182.

FIG. 184 illustrates a perspective view of the endoskeleton-cushion module of FIG. 180 or 181.

FIG. 185 illustrates a front transparent schematic showing current membrane or cushion transitionally shaped across the nose contour, and wherein the spiral shape positioned at the side of the nose provide a comfortable fit to a nose contour that can be geometrically changed rapidly. The spiral membrane region is shown in this Figure.

FIG. 186 illustrates a zoomed in view of the nose bridge contour region of a cushion showing the preferred distance between the left spiral membrane and the right spiral membrane positioned at the left side of the nose and the right side of the nose respectively.

FIG. 187 illustrates a zoomed-in partial front view of the endoskeleton-cushion attached to a frame, showing a preferred distance between the left spiral membrane and the right spiral membrane positioned at the left side of the nose and the right side of the nose respectively to accommodate a smaller and flatter nose shape.

FIG. 188 illustrates a zoomed-in partial front view of the endoskeleton-cushion attached to a frame, showing another preferred distance between the left spiral membrane and the right spiral membrane positioned at the left side of the nose and the right side of the nose respectively to accommodate a larger and flatter nose shape.

FIG. 189 illustrates a graph showing the nasal root breadth male distribution, showing one of the many anthropometric measurements used in physical anthropology to study human physical variation for facial reconstructions and for designing products like respiratory equipment. Regarding the distribution of nasal root breadth among males, it's important to note that such measurements can vary significantly across different populations and individuals due to genetic, environmental, and developmental factors. While the spiral geometry width can be adjusted for a bespoke mask such that different nose roots widths, which is the area where the nose meets the forehead, can be uniquely accommodated.

### DETAILED DESCRIPTION OF THE INVENTION

Preferred embodiments of the invention will now be described with reference to the accompanying drawings and non-limiting examples.

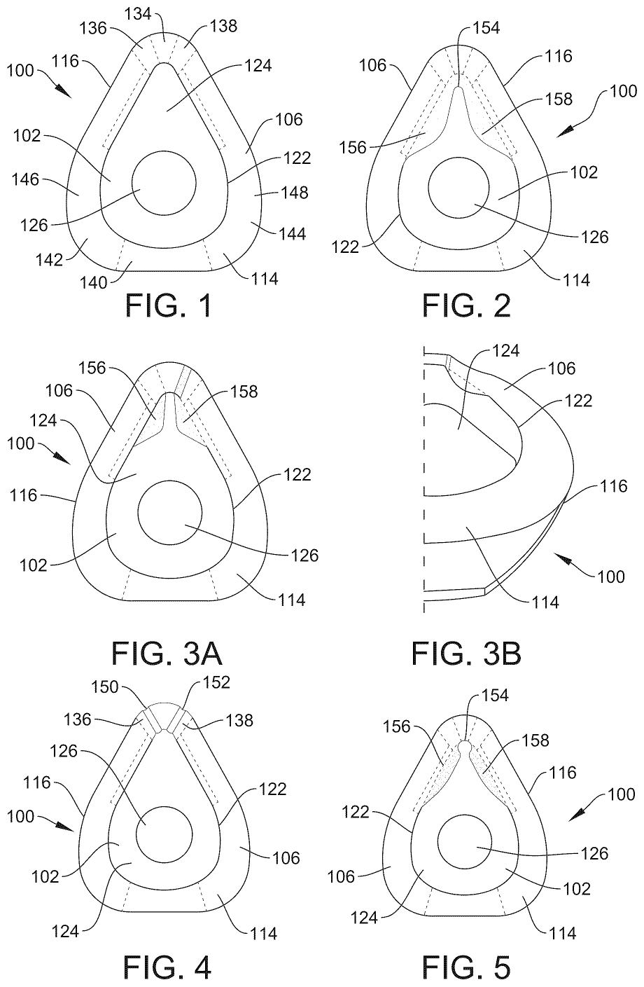

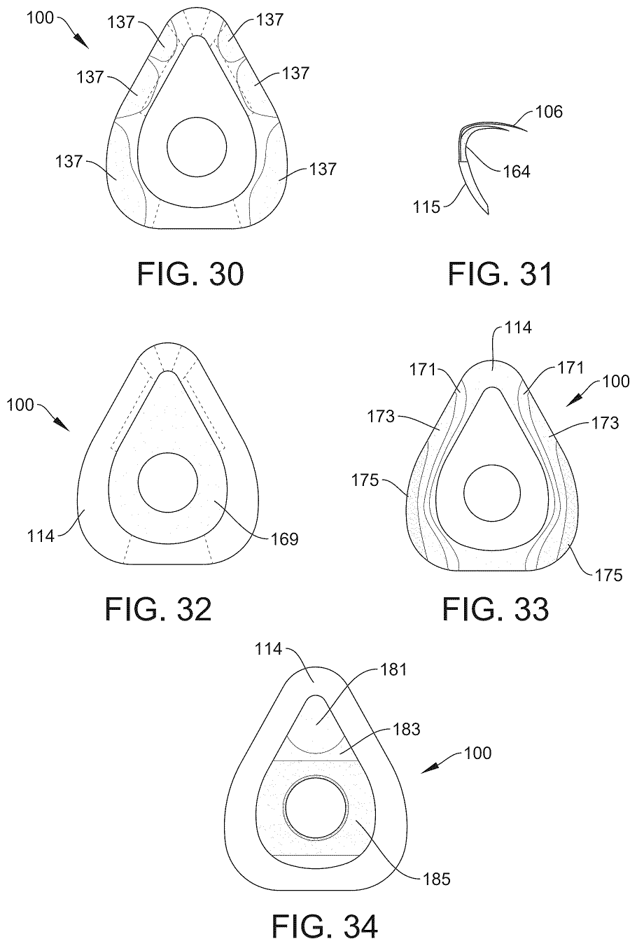

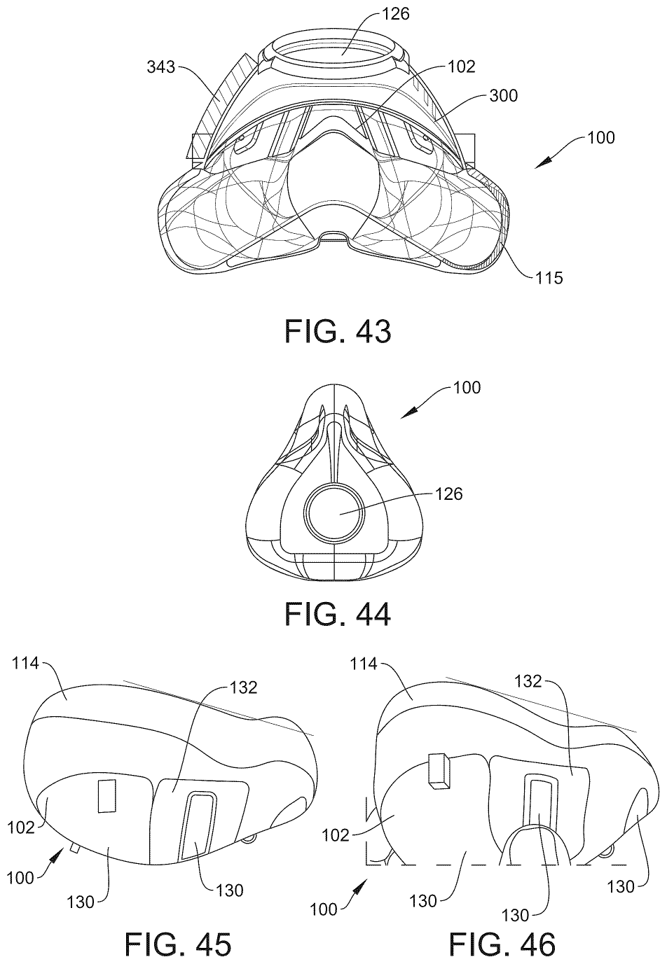

In an embodiment of a present invention, as illustrated in FIGS. 1 to 51C of a mask 100 that covers the nose and mouth regions. The mask 100 may have an encapsulated endoskeleton 102 and a cushion 114, which may be termed an endoskeleton-cushion module. FIG. 1 shows a back view of a mask 100 adapted for delivering pressurised air to a user, the mask 100 may comprise an endoskeleton 102 of a first resilient material 104 encapsulated to a lamina 106 of a second resilient material 108 forming a mask body 110 with a cavity 112. The second resilient material 108 may be relatively more flexible than the first resilient material 104. The lamina 106 or outer layer 106 may have a face contactable skirt portion 114 or a cushion 114 extending away from the endoskeleton perimeter 116. The skirt portion 114 may comprises an outer layer 106 and inner layer 118 for providing variable contoured thickness to the outer lamina segments 120 of the face contactable skirt portion 114. The face contactable skirt portion 114 may have a flexible cavity edge 122 defining a cavity opening 124 shaped to receive the nose and the mouth regions of the user's face. The encapsulated endoskeleton 102 may have an endoskeleton aperture 126 which is adapted to connect to an air delivery tube 500 or elbow 500. The thinner flexible cavity edge 122 may be adapted to wrap into the cavity 112, wherein when wrapped, the contoured thicker lamina segments 120 wraps and seals around the sides of the nose. The face contactable skirt portion 114 may comprise a nose bridge centre segment 134, a left 136 and a right side 138 of the nose segment, a chin segment 140, a left 142 and a right bottom corner segment 144, and a left 146 and right-side cheek segment 148. The thinner flexible cavity edge 122 at the bottom region may have a raised edge 128 in which the raised edge can wrap and seal around the chin and cheek.

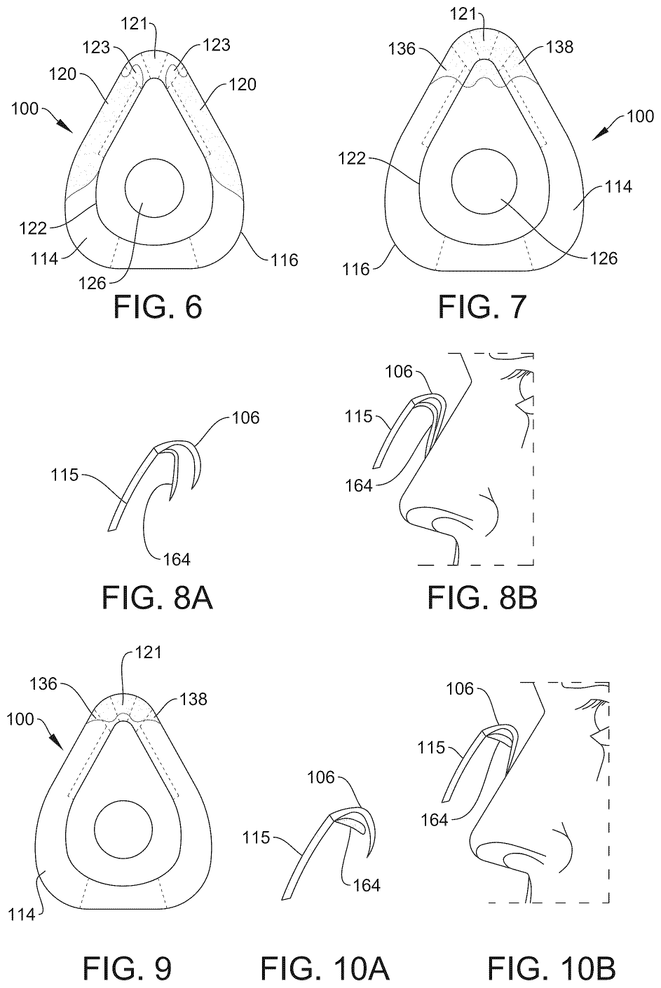

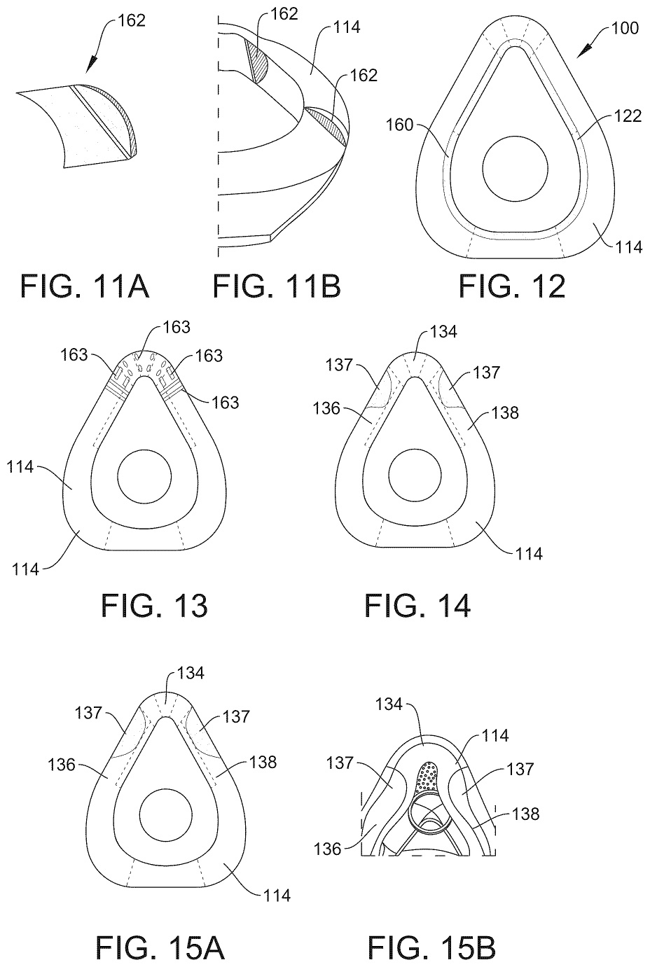

In another preferred embodiment of the skirt portion 114 as shown in FIG. 2, the mask 100 with the face contactable skirt portion 114 may have a narrow nose contour 154 that joins a left-side nose wing 156 and the right-side nose wing 158 via the nose bridge centre segment 134 or nose bridge centre region 134. This particular embodiment is for comfortably fitting users with smaller and flatter noses. Another preferred embodiment of the skirt portion 114 is shown in FIGS. 3A and 3B where the left-side nose wing 156 and the right-side nose wing 158 are individual wing portions for comfortably fitting users with larger noses. Another preferred embodiment of the skirt portion 114 is shown in FIG. 4, where the skirt portion 114 has thickened rib portions 150, 152 positioned at the left-side of the nose bridge segment 136 and the right-side of the nose bridge segment 138, which advantageously allows easy expansion and contraction of the lamina layer 106 or membrane layer 106 of the skirt portion 114 to accommodate wider facial geometries. Another preferred embodiment of the skirt portion 114 is shown in FIG. 5 where there is a stress relief radius 154 or a different curvature to the embodiment shown in FIG. 2, which also tailors the cushion membrane to expand easier for larger noses when a user is wearing the mask 100. Another preferred embodiment of the skirt portion 114 is shown in FIG. 6 where there may be a thin membrane 121 positioned behind the thick membrane ends 123 of the thick membrane 120 in the event of significant cushion compression. Another preferred embodiment of the skirt portion 114 is shown in FIG. 7 where there may be a thin seal membrane 121 partially covering under the skirt portion 114 from the left-side nose segment 136 and the right-side nose segment 138 and partially covering the cavity, and the secondary seal membrane 121 may cover under the outer layer 106 of the skirt portion 114 at the nose bridge side segments 136, 138. While a partial cross-sectional region of this secondary seal membrane is shown in FIG. 8A and how this is used when engaging to a person/user's nose in FIG. 8B, the secondary seal membrane 121 is tailored to wrap and seal to different known challenging nose geometries. Another preferred embodiment of the skirt portion 114 is shown in FIG. 9 where there may be a thin seal membrane 121 partially covering under the skirt portion 114 from the left-side nose segment 136 and the right-side nose segment 138, and the secondary seal membrane 121 may cover under the outer layer 106 of the skirt portion 114 at the nose bridge side segments 136, 138 without partially covering the cavity. While a partial cross-sectional region of this secondary seal membrane is shown in FIG. 10A and how this is used when engaging to a person/user's nose in FIG. 10B, the secondary seal membrane 121 is also tailored to wrap and seal to different known challenging nose geometries. Another preferred embodiment of the skirt portion 114 is shown in FIG. 11B where there may be provided a thickening ribs segments 162 at the cheek segments 146, 148 under the skirt portion 114. The thickening rib segment 162 is shown in FIG. 11A. Another preferred embodiment of the skirt portion 114 is shown in FIG. 12 where the flexible cavity edge 122 defining a cavity opening 124 has a polished edge 161 for an improved seal around the edge 122. Another preferred embodiment of the skirt portion 114 is shown in FIG. 13 where specific or selective polished areas 163 which does not cover the entire skirt portion 114 so as to improve seal contact without fully sacrificing comfort. Another preferred embodiment of the skirt portion 114 is shown in FIG. 14, where there may be small localised dual walls 137 at one side of the nose segment 136 and the other side of the nose segment 138. The localised dual walls are localised only at the sides of the nose to improve the seal around the nose. In this contoured localised wall shape, the cheek compression does not affect the mask's performance. Another preferred embodiment of the skirt portion 114 is shown in FIGS. 15A and 15B where the thick membrane 137 has a specific contour shape to allow the region to wrap and seal against a person's nose.

The mask performance could be improved by the inclusion of grooves create weaker points to allow the mask to selectively flex. The grooves may be preferable to corrugations or concertina arrangements as the grooves may provides consistent wall section for strength whilst providing flexibility around the corrugations that behave like integral hinges.

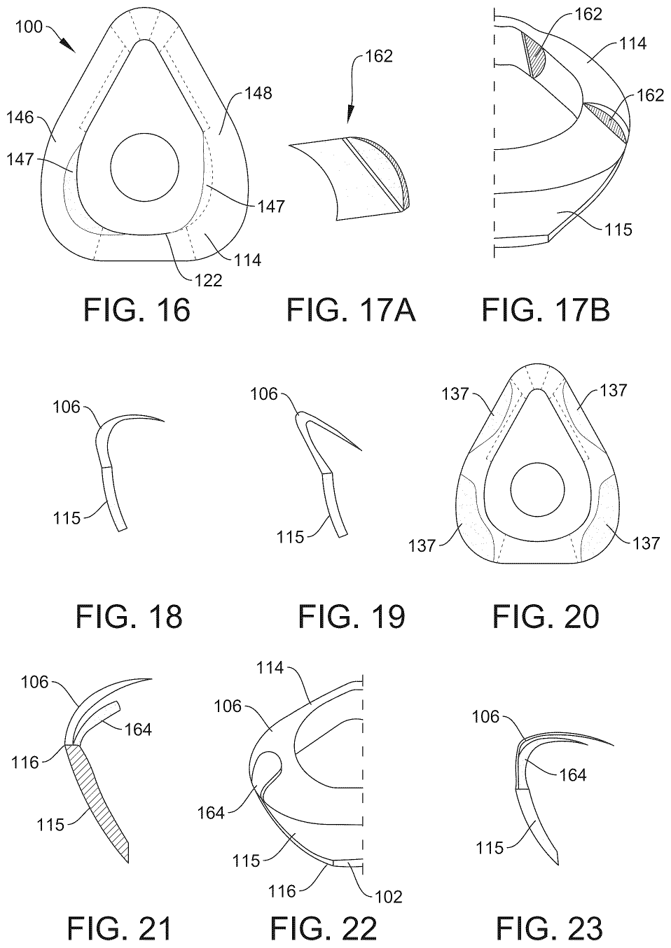

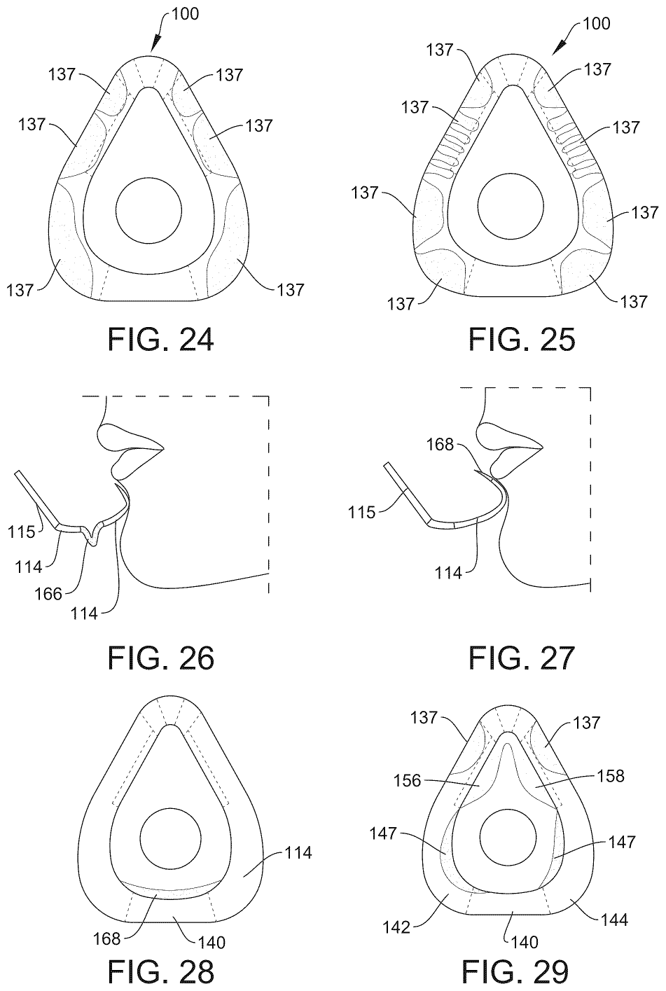

Another preferred embodiment of the skirt portion 114 is shown in FIG. 16 where the outer lamina 106 or outer layer 106 has an extended thin membrane 147 inwards to the cavity so as to increase the compliant section of the cushion membrane to reduce mouth leak at those regions. Another preferred embodiment of the skirt portion 114 is shown in FIGS. 17A and 17B, shows other thickened ribs 162 positioned in other segments under the skirt portion 114 to provide structural support to those segments under compression stress when in use. Another preferred embodiment of the skirt portion 114 is shown in FIG. 18, where a partial side profile of a curled membrane 106 or a wrapped skirt portion is joined to the buffer membrane portion 115 for improving the likelihood of the skirt portion 114 rolling. The curled membrane 106 configuration is preferred over the folded membrane configuration as shown in FIG. 19 as folded membranes are likely to bend or crushed. Another preferred embodiment of the skirt portion 114 is shown in FIG. 20 where different thickening regions are placed at the nose segment and the cheek segments to optimise stiffness of the skirt portions at those segments. The thickening regions may have a predetermined shape and thickness to optimise seating in the most comfortable place on the cheek. Another preferred embodiment of the skirt portion 114 is shown in FIG. 21, where the inner membrane 164 can be a simple cantilever beam design to further provide thickness under the skirt portion 114, in which the contextual view of the inner membrane is positioned in view of the mask is shown in FIG. 22. Another preferred embodiment of the skirt portion 114 is shown in FIG. 23, in which the inner membrane 164 may have a tapered beam design to provide a gradual or gradient of thickness under the skirt portion 114. Another preferred embodiment of the skirt portion 114 is shown in FIGS. 24 and 25 where multi-segment dual walls are strategically positioned to provide the optimal and more personalised fit. It may be appreciated that as different users have different facial geometries, the regions of thickened areas and shapes can be adjusted to accommodate for complex facial regions.

Another preferred embodiment of the skirt portion 114 is shown in FIG. 26 where a chin gimble 166 for providing sealing support to the chin region of the person and an extended chin membrane 168, as shown in FIG. 27 can be used for increasing the compliance for jaw drop to reduce jaw leak, in which the extended chin membrane 168 covers the edge of the skirt portion 114 of the chin region 140. As shown in FIGS. 29 to 31, the mask 100 may use any of the above-described features alone or in combination to produce a personalised fit and comfort to the user.

Another preferred embodiment of the endoskeleton 102 is shown in FIG. 32, where the endoskeleton 102 is made from a rigid plastic so as to provide structural integrity to the endoskeleton-cushion module. The rigid plastic made be a material not limited to polycarbonate and nylon. This embodiment allows forces to be transferred to the edge of the cushion membrane equally. Another preferred embodiment of the skirt portion stiffness 114 is shown in FIG. 33 showing the contoured region indicated 114 for the nose and chin segments are highly soft and compliant. The next contoured regions indicated at 171 have low stiffness, 173 have medium stiffness, and 175 have high stiffness more particularly at the sides of the cheek regions to provide support in a compressive stressed area of the mask. This embodiment varies the force it applies to the face in these areas. Another preferred embodiment of the endoskeleton 102 region stiffness is shown in FIG. 34 where the regions indicated in 181 is low stiffness, 183 is medium stiffness, and 185 is high stiffness more particularly surrounding the endoskeleton aperture 126. It is preferred to have the force applied by the cushion variable around the membrane so the force required by the endoskeleton 102 will also vary and hence the stiffness of the chassis can vary without impacting cushion performance.

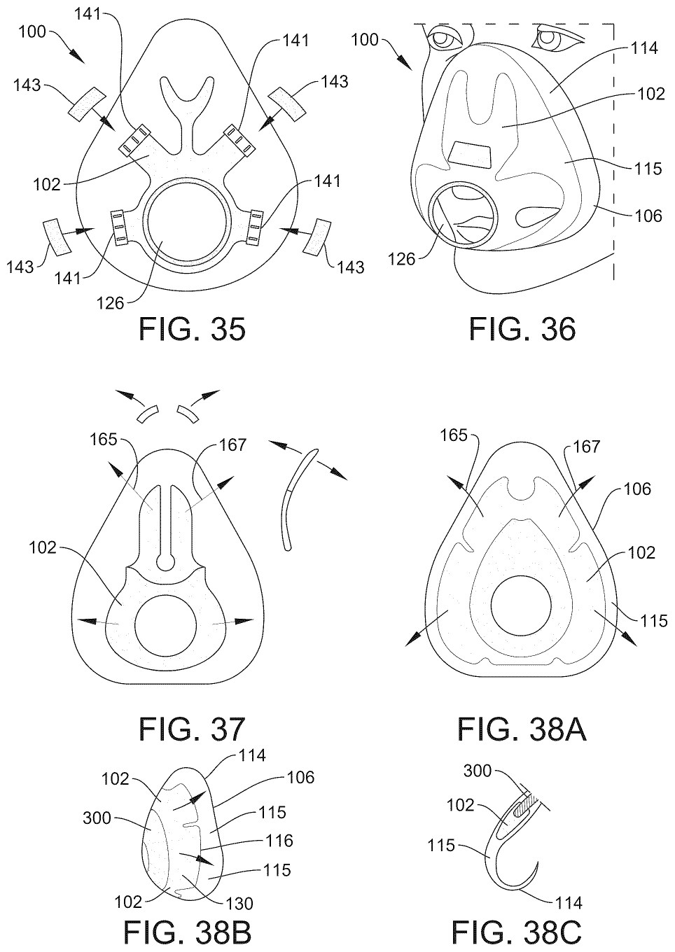

Another preferred embodiment of the endoskeleton 102 is shown in FIG. 35, where the endoskeleton 102 may have a mechanical interlock mechanism 141 for allowing straps 143 to be engaged and seal with the mask 100. Another preferred embodiment of the endoskeleton 102 is shown in FIGS. 37 to 38B where the shape of the endoskeleton can be varied to allow forces (indicated by direction of black arrows) to flex, and FIG. 38C shows a partial side schematic view of the embodiment of FIG. 38B.

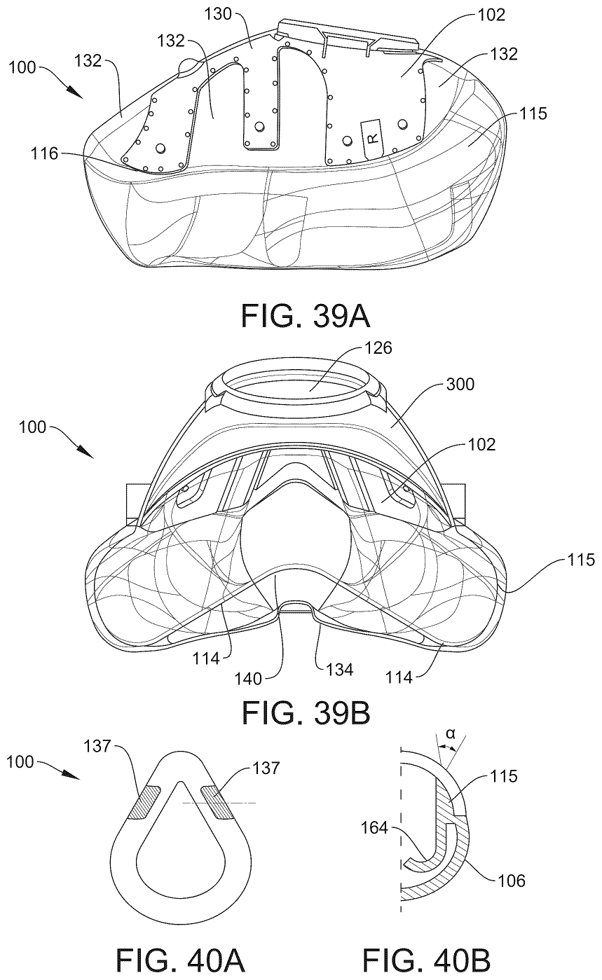

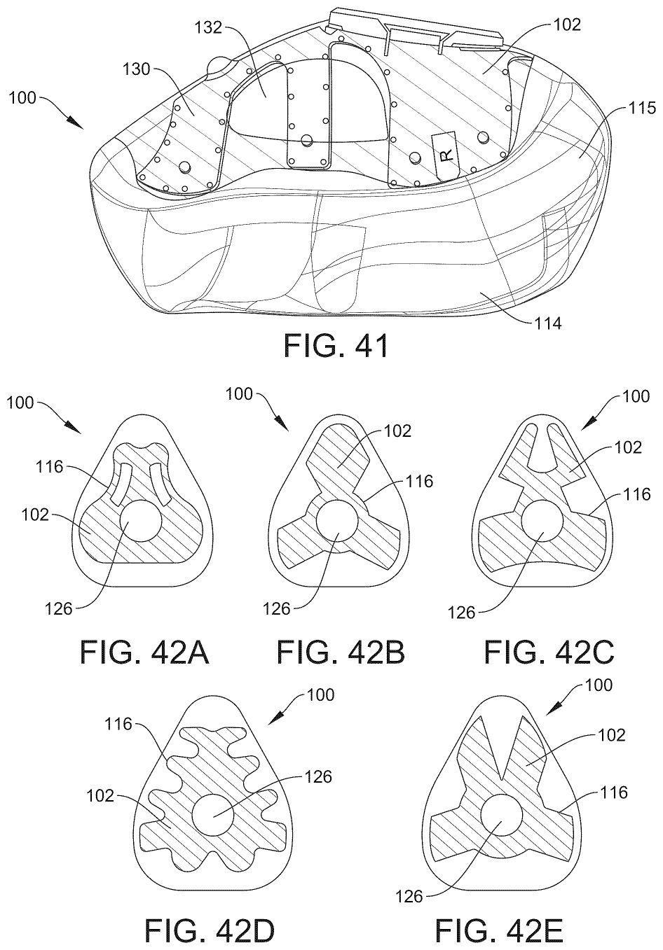

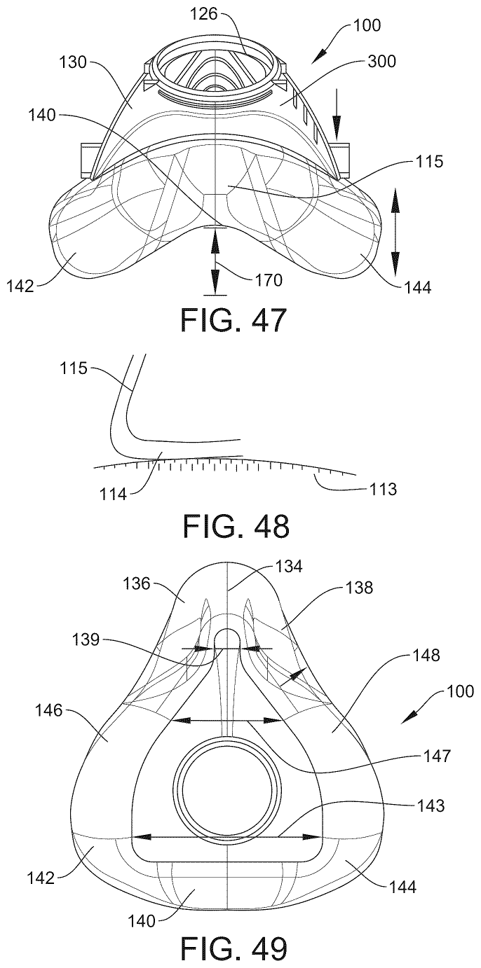

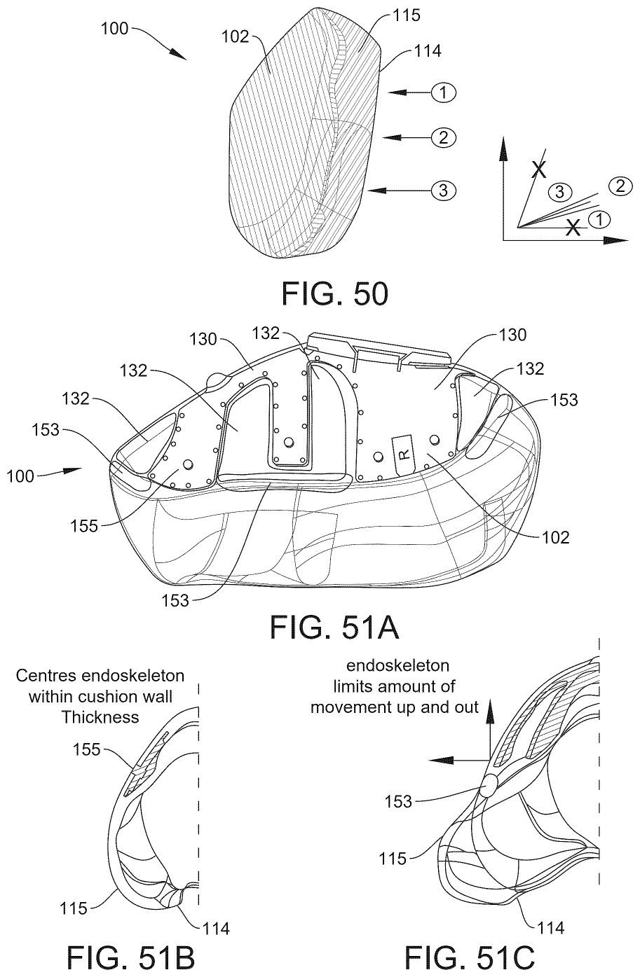

Another preferred embodiment of the endoskeleton 102 is shown in FIGS. 39A, 39B where the encapsulated endoskeleton 102 may have a major segment 130 and at least one minor segment 132. The inner membrane 164 may be angled to direct the force normal to nose, as shown in FIG. 40B, which may confer a reduction of drag and enhance the overall efficiency of the mask particularly in situations where streamlined airflow is crucial and that the angling of the inner membrane might enhance the structural integrity of the object. By aligning the force normal to nose, it can withstand and distribute loads more effectively. Another preferred embodiment of the endoskeleton 102 is shown in FIG. 41, the minor segments 132 can be positioned in the overlayed region and the shape of the major segment 130 can be adjusted accordingly. Another preferred embodiment of the endoskeleton 102 is shown in FIGS. 42A to 42E, the endoskeleton structures 102 is not limited to any of the illustrated shapes provided as an example. Another preferred embodiment of the mask 100 is shown in FIG. 41 where it shows how the exoskeleton 300 is positioned over an encapsulated endoskeleton 102 with a cushion 115 showing the curvature. The exoskeleton 300 may have a framework 343 or a frame arm 353 that is for providing a linkage to a headgear 600. As shown in FIGS. 45 to 46, the major segment 130 and the minor segments 132 are of the same material, and wherein the at least one minor segment 132 are hingedly connected 133 to the major segment 130, FIG. 44 is the back view of the FIGS. 45 to 46 and as such showing how the face contactable skirt portion 114 is shaped for the embodiment shown by FIGS. 45 to 46. As shown in FIG. 47, the arrow at the exoskeleton 300 would allow the headgear load to transfer vertically into the side of the mouth, and reduce horizontal component to headgear force and the buffer lamina portion 115 may have a sufficient depth to allow cushioning to the face. And that the ‘V’ shape of the buffer lamina portion 115 may advantageously allow the skirt portion 114 to contact the chin earlier, and when the ‘V’ shape is of a sufficient depth (as indicated by the arrows), the sufficient depth may not put undue pressure on the chin and allow for comfortable mouth movement. Another preferred embodiment of the skirt portion 114 is shown in FIG. 49 where the narrow cushion opening 139 allows to seal against the narrow noses, nose width 147 being flexible enough to curl away, and a mouth width 143 for fitting majority of mouth widths. As shown in FIG. 48, the distal end of the membrane biased so that it will readily contact skin. As shown in FIG. 50, the force deflection curves in the indicated regions of the mask is preferably similar across thick regions. Another preferred embodiment of the endoskeleton 102 is shown in FIG. 51A where it may be preferable to have a silicone band 153 at the minor segment 132 edge or perimeter, which acts as a tensioner so as to transfer force in alternative directions.

As shown in FIG. 4, the left-side nose bridge segment 136 may have a first thickened rib 150, and the right-side bridge segment 138 may have a second thickened rib 152. The first and second thickened ribs allows its shape to be maintained and seals at the nose bridge segment 136 when subjected to pressurised air delivered by the air delivery tube 500. As shown in FIG. 3A, the flexible cavity edge 122 between the left side nose segment 136 and the right-side nose segment 138 via the nasal centre segment 134 comprises a biased contour portion 154 connecting between a left-side nose wing 156 and a right-side nose wing 158. The biased contour 157 may allow for an increased seal with the nasal bridge while the nose wings 156, 158 increase the surface area allowing for reducing air blow out from sides of nose. In another embodiment, as shown in FIG. 5, the biased contour 157 is a stress relief radius 159.

As shown in FIG. 12, the flexible cavity edge 122 may be a polished edge 160 around the edge of the face contacting portion 114. For example, the polished edge may be a minimum of 3 mm. As shown in FIGS. 14, 15A, 15B, and 20, the left side nose segment 136 and the right-side nose segment 138 may each comprise a contoured segment 137 thicker relative to the nose bridge centre segment 134. As shown in FIGS. 11A and 11B, the left-side cheek segment 146 and the right-side cheek segment 148 may each comprise stiffening ribs 162 for maintaining the shape of the skirt portion 114 when subjected to pressurised air delivered by the air delivery tube 500. As shown in FIG. 16, the mask 100 may have a thin lamina or thin membrane layer 147 at the edge of the skirt portion 114 at the cheek segments 146, 148, the bottom corner segments 142, 144, and the chin segment 140 due to stiffness not being required at the edges so the thin membrane layer can be thinned earlier to resolve mouth leak. As shown in FIGS. 21 to 25, in another preferred embodiment, a buffer lamina portion 115 may be positioned between the endoskeleton perimeter 116 and the face contactable skirt portion 114. The outer lamina 106 of the face contactable skirt portion 114 and a cantilever inner lamina 164 may each extend from the buffer lamina portion 115 at the same location, wherein the cantilever inner lamina 164 has a predetermined thickness. For comfort, the outer lamina 106 is frosted and wherein the outer lamina is made of silicone. As shown in FIGS. 26 and 28, the mask 100 may further comprise a chin gimble 166 and as shown in FIGS. 27 and 29, the flexible cavity edge 122 may comprise an extended chin lamina 168 for reducing jaw leak at the chin. As shown in FIG. 47, the bottom side profile of the skirt portion 114 has a ‘V’ shape 170 for allowing the chin segment 140 of the skirt portion 114 to contact the chin earlier, when in use. As shown in FIG. 49, the chin region may have a thickness between 0.20 mm to 0.45 mm, more specifically 0.35 mm thin lamina for reducing the force on the chin and to accommodate mouth movement. The endoskeleton 102 may be made from a rigid plastic material selected from the group of: polycarbonate, and polyamide (nylon). It may be appreciated that other materials similar to the ones listed may also be suitably used.

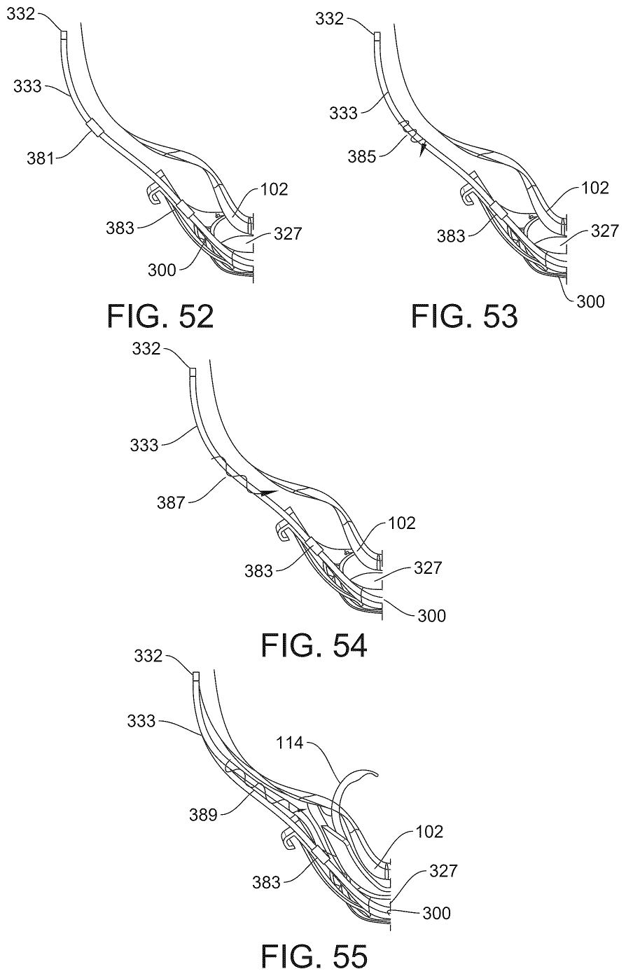

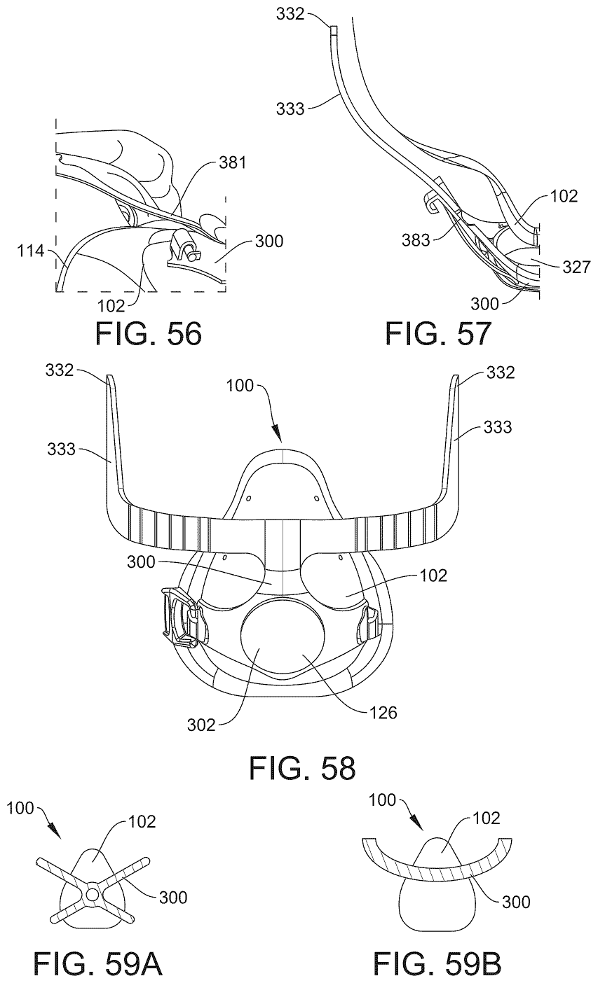

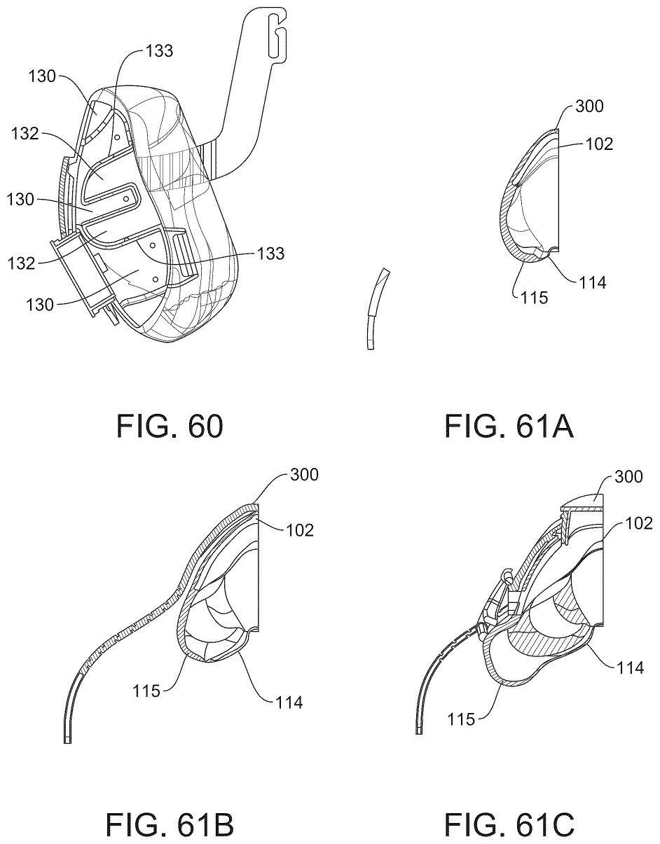

As shown in FIG. 58, the mask 100 may further comprise an exoskeleton 300 securable to the encapsulated endoskeleton 102. The exoskeleton 300 may have an exoskeleton aperture 302 with an aperture profile shape congruent to the endoskeleton aperture 126, wherein when secured, the exoskeleton aperture 302 may be positioned over the encapsulated endoskeleton aperture 126 such that the air delivery tube may be connectable to the cavity 112 through the exoskeleton aperture 302 and the encapsulated endoskeleton aperture 126. FIG. 52 illustrates a partial perspective view of a framework or an exoskeleton 300 of a preferred embodiment for mounting to an endoskeleton 102 of any of the previously described embodiments. In this embodiment, this framework 300 has a first hinge portion 381 and a second hinge portion 383 positioned along a left/right-side of the framework 300. The first hinge portion 383 and the second hinge portion 381 are spaced away relatively from each other. And the hinge portions 381, 383 are positioned between the central aperture 327 or exoskeleton aperture 327 of where the air delivery tube 500 is to be connected and the headgear mounting means 332. FIG. 53 illustrates a partial perspective view of another framework of another preferred embodiment similar to the framework of FIG. 52, in which the second hinge portion 381 is replaced by a corrugated portion 385 along a short predetermined length along the framework arm 333. The corrugated portion 385 having a first predetermined wavelength. The first hinge portion 383 and the corrugated portion 385 are spaced away relatively from each other. And the corrugated portion 385 positioned between the first hinge portion 383 and the headgear mounting means 332 at the end of the framework. This particular corrugation gives better hugging where the face curvature changes the most. FIG. 54 illustrates a partial perspective view of another framework of another preferred embodiment similar to the framework of FIG. 53, in which the corrugated portion 387 is also along a short predetermined length along the framework arm 333. The corrugated portion 387 having a second predetermined wavelength. The corrugations may also provide an optional benefit of having a spring constant higher than the headgear. This may allow a small amount of stretch wherein the mask contacts the bedding such as side sleeping to reduce the effect on the cushion seal. The second predetermined wavelength is relatively longer than the first predetermined wavelength of the framework as depicted in FIG. 53. The first hinge portion 383 and the corrugated portion 387 are spaced away relatively from each other. And the corrugated portion positioned between the first hinge portion 383 and the headgear mounting means 332 at the end of the framework. This bigger corrugation pitch along a longer distance and it provides a more gradual bending, better force transfer and perform less like a hinge. FIG. 55 illustrates a partial perspective view of another framework of another preferred embodiment, in which this embodiment has a corrugated portion 389 positioned between the central aperture 327 and the headgear mounting means 332 at the end. This framework advantageously allows to contact the face earlier as the longer corrugation and bigger pitch hugs the face better, and has less external force to break seal in dynamic movements. FIG. 56 illustrates the framework of FIG. 55 in use with a person's face. FIG. 57 illustrates the framework having a combination of a hinge portion 383 to allowably flex at least 13 mm horizontally on each side.

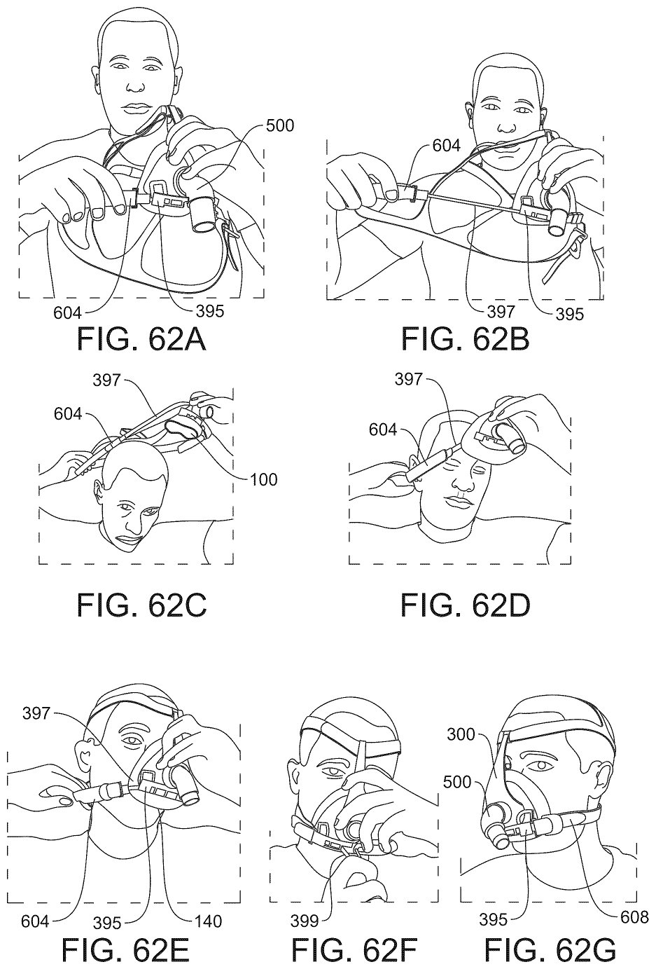

In another embodiment, as shown in FIGS. 62A to 62G of a person putting on and securing a mask with a headgear attached, there may be an assembled mask 100 with exoskeleton 300 mounted to an endoskeleton 102, while the elbow 500 is attached through the exoskeleton aperture and the endoskeleton aperture. To assist users in not losing multiple parts of the mask, it can be useful to have a headgear 600 that is attached to the exoskeleton 300. The securing means 395 at the lower portion of the exoskeleton 300 may have elastic members 397 in connection to the left and right lower portions of the headgear 604, 608. As shown in FIG. 62F, there is a tightening mechanism 399 positioned at the front to secure this mask and headgear embodiment when comfortably positioned

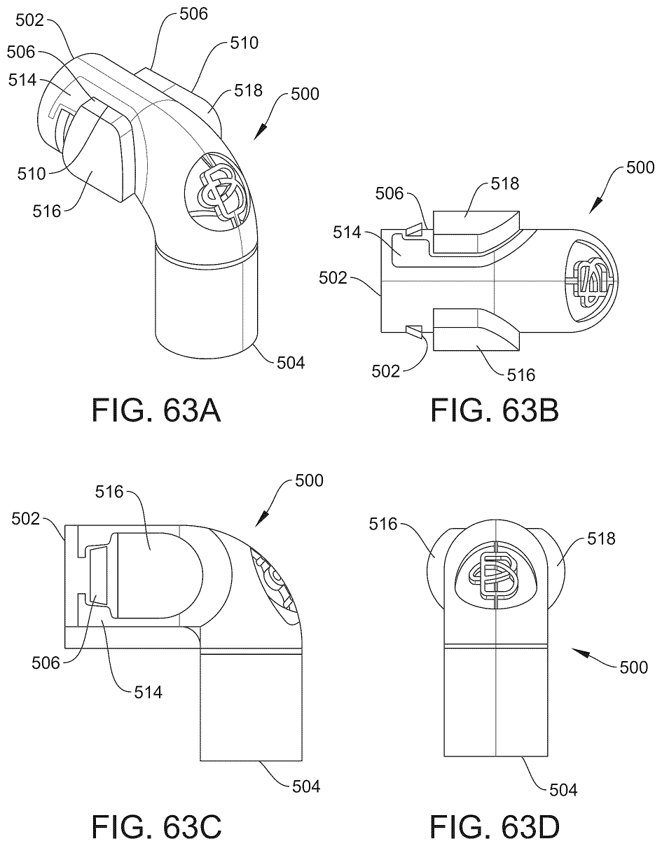

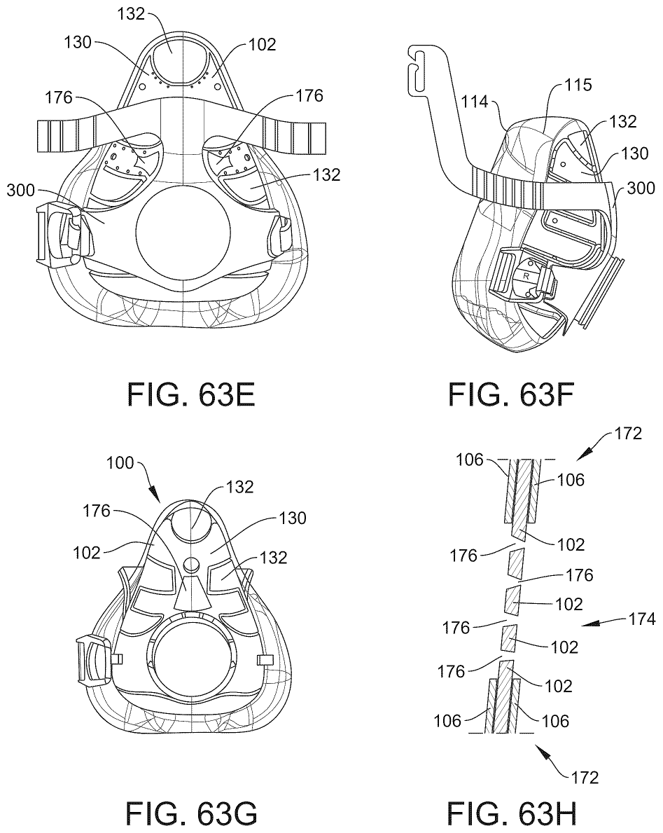

As shown in FIGS. 63A to 63D, the air delivery tube 500 or elbow 500 may have a first end 502 and a second end 504. The first end 502 may have a snap connection 506 for sealingly interlocking to the exoskeleton aperture 302 and the encapsulated endoskeleton aperture 126. The second end 504 may be attachable to a Continuous Positive Airway Pressure (CPAP) apparatus 508 (not shown). As shown in FIGS. 63A and 63B, the first end 502 of the air delivery tube 500 may comprise a snap release mechanism 510 positioned inside the exoskeleton 300, wherein the snap release mechanism 510 is facilitated by rotating about a fulcrum 512 and deforming an elastic thermoplastic elastomer 514 which is positioned between the first release button 516 and the second release button 518, wherein the snap release mechanism 510 is triggered by squeezing the first release button 516 and the second release button 518 towards each other, which deforms the elastic thermoplastic elastomer 514 allowing the first end 502 of the elbow 500 to be disengageable of the apertures 126, 302 of the mask 100. The disengagement is through loosening and allows the elbow 500 to rotate relative to the apertures 126, 302 to disengage.

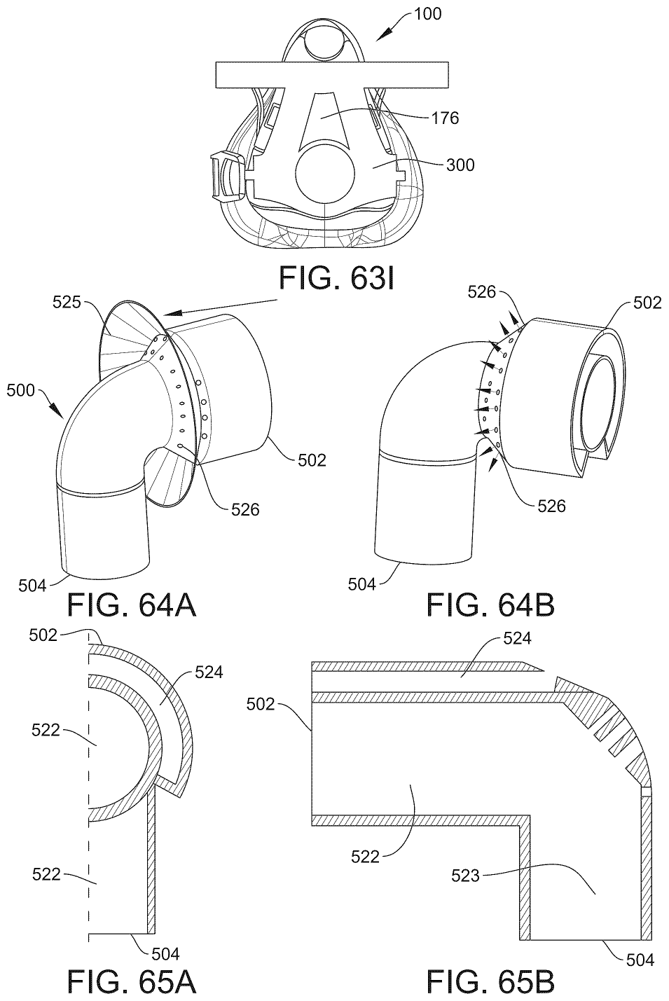

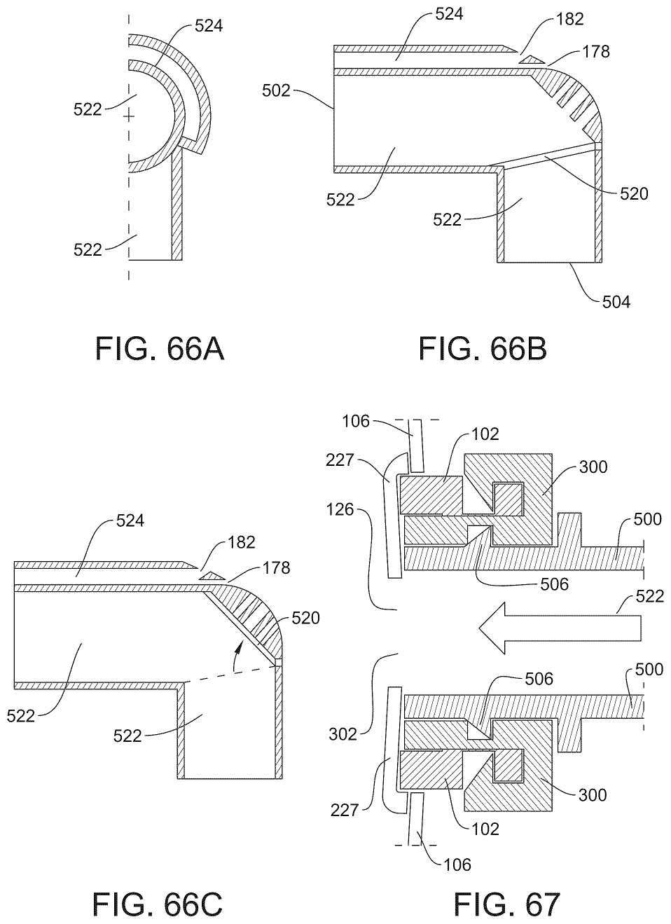

As shown in FIG. 66B, the air delivery tube 500 may further comprise an anti-asphyxia valve (AAV) 520 positioned in an air inflow channel 522 which is positioned and in fluid connection between the first end 502 and the second end 504 of the air delivery tube 500. As shown in FIGS. 65A and 65B, the air delivery tube may comprise a CO2 washout channel 524, which is an independent channel 524 to the air inflow channel 522 or a CPAP airway 522 from the second end 504 to the first end 502. As shown in FIGS. 64A and 64B, the air delivery tube 500 may comprise annular vent holes 526 for directing air from the CO₂ washout channel 524 in a conical shape 525 out of the air delivery tube 500. Another preferred embodiment (not shown) of the elbow 500 that is connectable to the endoskeleton and exoskeleton aperture of the mask, in which this mask may be a round hole without a lip seal. The first end of the elbow may have grooves in the interfacing surfaces of the endoskeleton and exoskeleton aperture, in which the grooves can act as an independent channel for venting out air or acting as CO₂ washout channel.

In another preferred embodiment, as shown in FIG. 63G, the endoskeleton 102 may comprise an encapsulated area 172 and a non-encapsulated area 174, wherein a vent hole array 176 may be positioned at the non-encapsulated area 174. As shown in FIGS. 63E and 63F, the vent hole array 176 may have a profile that is at least one selected from the group of: front facing 178, angled out to the side 180, tapered 182, and non-tapered 184.

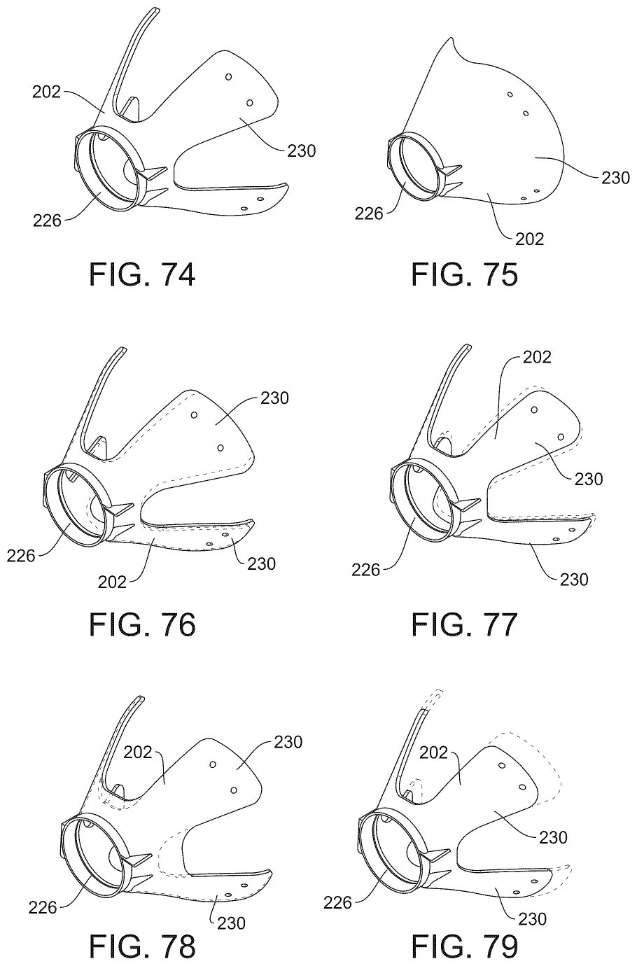

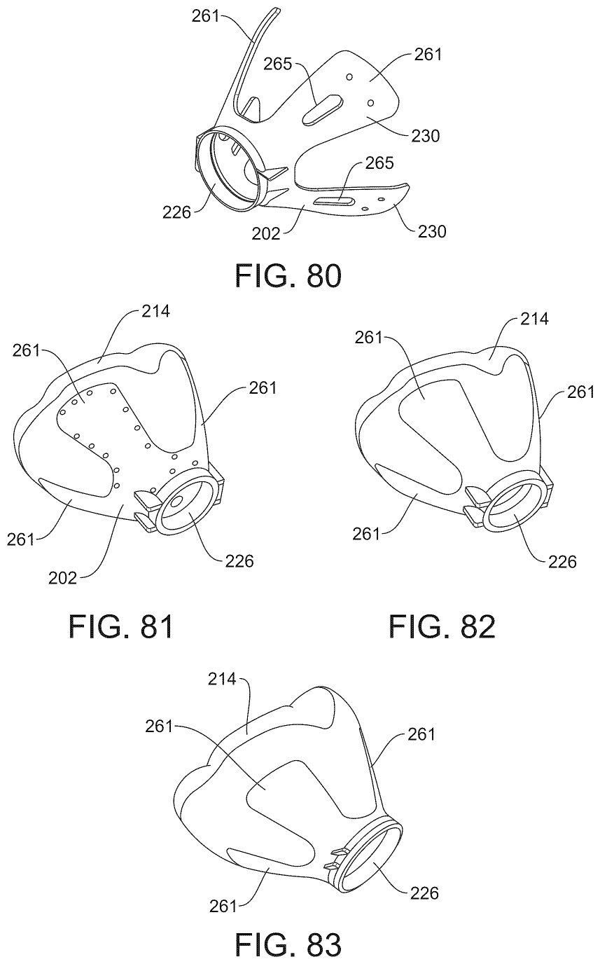

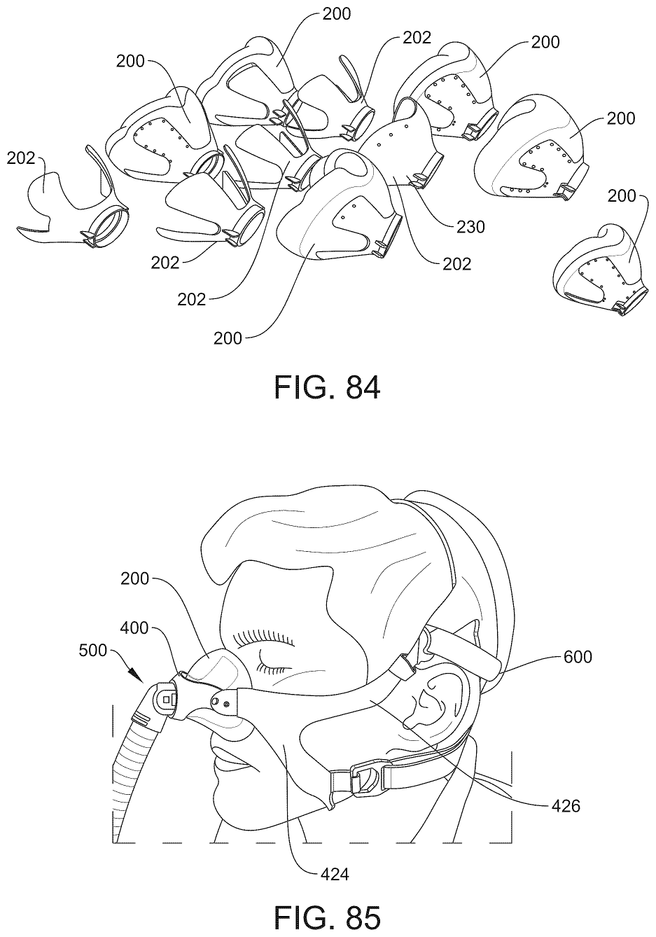

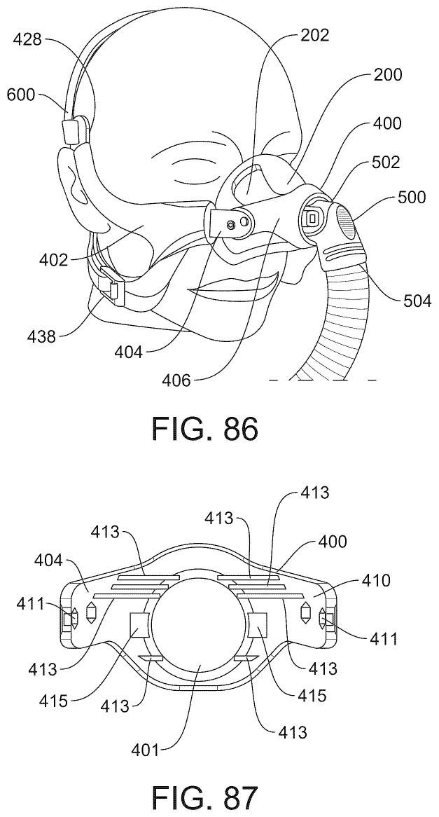

In another preferred embodiment of the mask, as shown in FIGS. 74 to 79, there may be a mask 200 adapted for delivering pressurised air to a user, the mask 200 may comprise an endoskeleton 202 of a first resilient material 204 encapsulated to a lamina 206 of a second resilient material 208 forming a mask body 210 with a cavity 212. The second resilient material 208 may be relatively more flexible than the first resilient material 204. The lamina 206 or outer layer 206 may have a face contactable skirt portion 214 or a cushion 214 extending away from the endoskeleton perimeter 216. The skirt portion 214 may comprises an outer layer 206 and inner layer 218 for providing variable contoured thickness to the outer lamina segments 220 of the face contactable skirt portion 214. The face contactable skirt portion 214 may have a flexible cavity edge 222 defining a cavity opening 224 shaped to receive the nose region of the user's face. The encapsulated endoskeleton 202 may have an endoskeleton aperture 226 with a lip seal 227 which is adapted to connect to an air delivery tube 500 or elbow 500. The thinner flexible cavity edge 222 may be adapted to wrap into the cavity 212, wherein when wrapped, the contoured thicker lamina segments 220 wraps and seals around the sides of the nose. The face contactable skirt portion 214 may comprise a nose bridge centre segment 234, a left 236 and a right side 238 of the nose segment, an upper lip segment 240, a left 242 and a right bottom corner segment 244, and a left 246 and right-side cheek segment 248. The thinner flexible cavity edge 222 at the bottom region may have a raised edge 228 in which the raised edge can wrap and seal around the upper lip and cheek.

As shown in FIGS. 81 to 83, the encapsulated endoskeleton 202 may have a major segment 230 and at least one minor segment 232. The major segment 230 and the minor segments 232 are not of the same material, and wherein the at least one minor segments 232 are connected to the major segment 230. The flexible cavity edge 222 between the left side nose segment 236 and the right-side nose segment 238 via the nasal centre segment 234 comprises a biased contour portion 254 connecting between a left-side nose wing 256 and a right-side nose wing 258. The biased contour 257 may allow for an increased seal with the nasal bridge. In another preferred embodiment, while the nose wings 256, 258 may increase the surface area allowing for reducing air blow out from sides of nose. In another preferred embodiment, the biased contour 257 may be a stress relief radius 259. The major segment 230 may have elongate structural members 261 for providing support to the face contactable skirt portion 214 of at least one segment selected from the group of: the left 236 and right side 238 of the nose segment, the upper lip segment 240, the left 242 and right bottom corner segment 244, and the left 246 and right-side cheek segment 248.

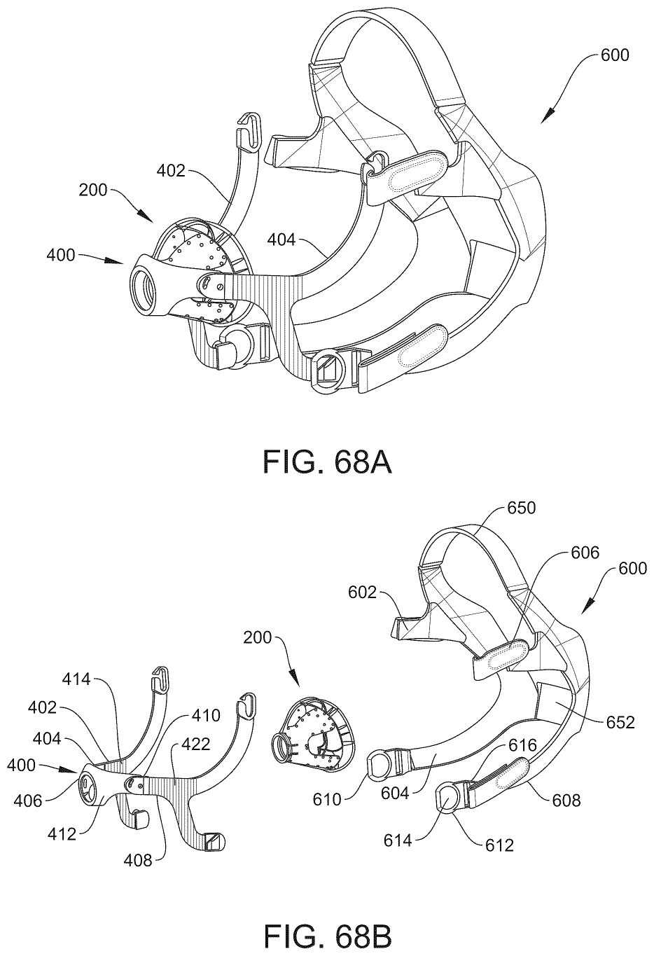

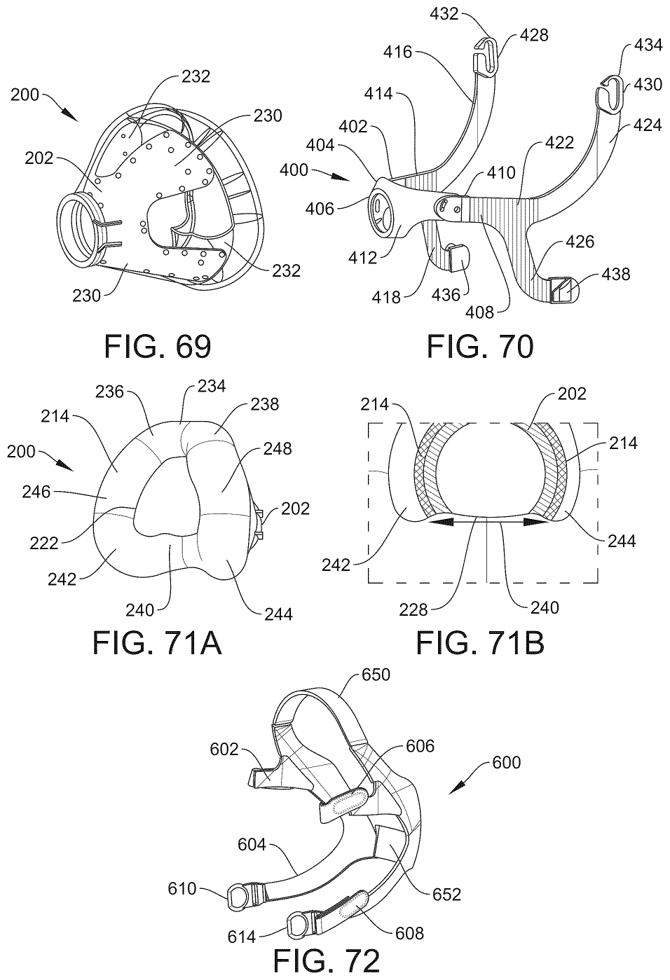

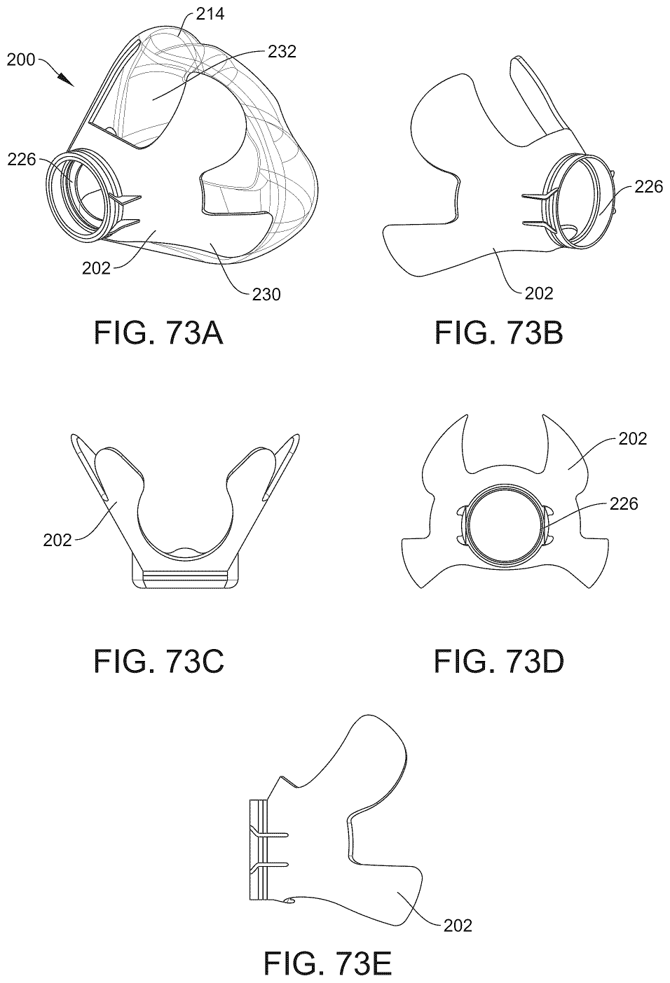

In another preferred embodiment, as shown in FIGS. 68A, 68B, 70, the mask 200 may further comprise an exoskeleton 400 which is mountable to the front of the endoskeleton 202. Preferably, the exoskeleton aperture 401 is of a shape that is congruent to the endoskeleton aperture 226 and when mounted, the exoskeleton aperture 401 is over the endoskeleton aperture 226. The first frame arm 402 may have a first exoskeleton connector 404 securable to a first side of the exoskeleton 406, and a second frame arm 408 may have a second exoskeleton connector 410 securable to a second side of the exoskeleton 412. The first side of the exoskeleton 406 may be positioned opposite or the other side to second side of the exoskeleton 412. The first frame arm 402 may have a first bifurcate portion 414 that branches to a first upper frame arm portion 416 and a first lower frame arm portion 418; and the second frame arm 420 may have a second bifurcated portion 422 that branches to a second upper frame arm portion 424 and a second lower frame arm portion 426. The first upper frame arm portion 416 may have a first upper headgear securing means 428 and a second upper frame arm portion 420 may have a second upper headgear securing means 430. As shown in FIGS. 68A and 68B, the first upper headgear securing means 428 and the second upper headgear securing means 430 may each be an open ‘C’-shaped clip 432, 434. The first lower frame arm portion 418 may have a first hook 436 and the second lower frame arm portion 426 may have a second hook 438, in which the first hook 436 is for securing the first lower headgear portion, and the second hook 438 is for securing the second lower headgear portion.

As shown in FIG. 87, the first exoskeleton connector 404 and the second exoskeleton connector 410 are each having a slot of a small angle of articulation 411 such a degree range between 15° to 25° with respect to the longitudinal axis of the frame arm. More preferably and optimally, the angle of articulation is 20° in which the slot with a small angle of articulation can help reduce friction between components. With a smaller angle, there is less sliding motion between surfaces, resulting in lower frictional forces. This can contribute to improved efficiency and decreased wear and tea. The small angle of articulation can exhibit greater stability. This can be advantageous in maintaining the structural integrity of a mechanism and preventing unintended movements or vibrations. It also requires less space to achieve a given range of motion. To support the exoskeleton 400, there may be provided elongate ribs 413 and the positioning of the snaps 415 for mounting to the endoskeleton cushion module 200 or mask 200.

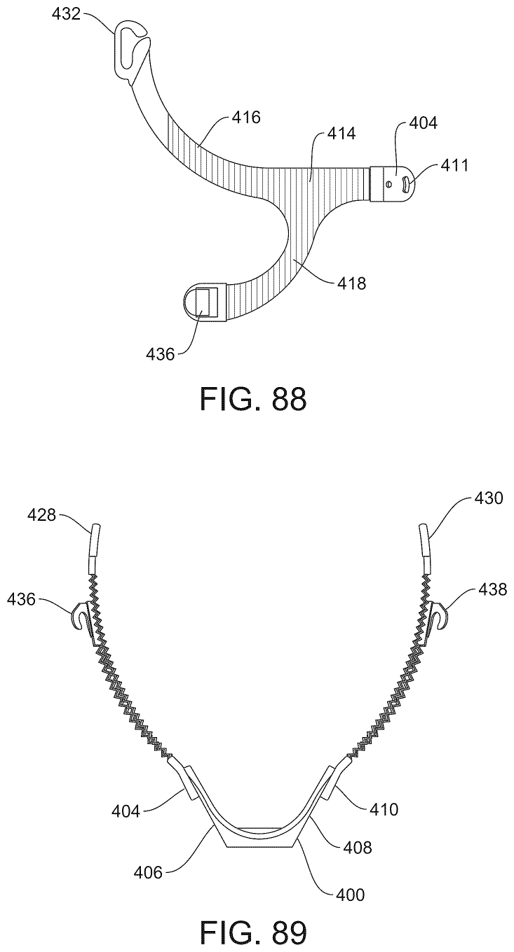

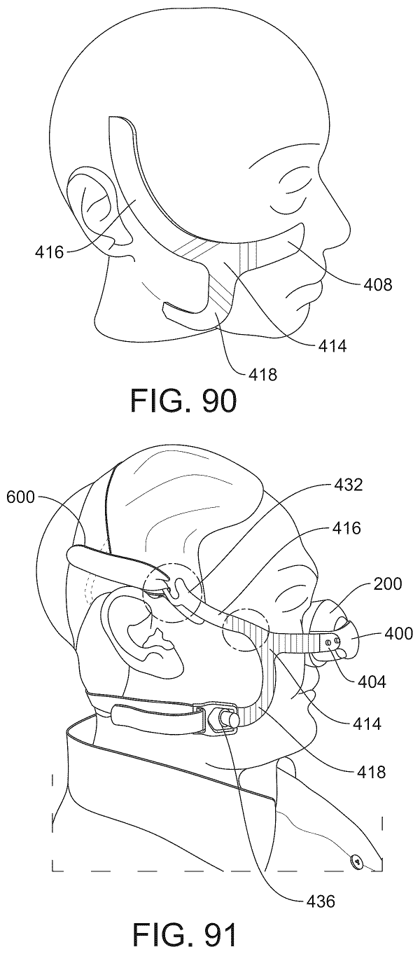

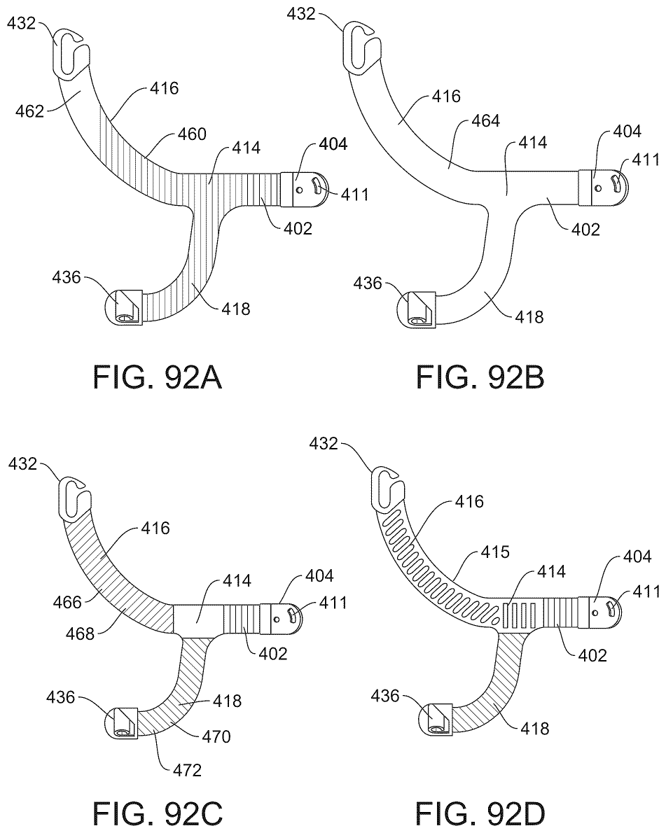

In another preferred embodiment, as shown in FIG. 88, each of the frame arms may have vertically corrugated portions 460 or sections positioned along the longitudinal axis of the frame arm between the connecting ends. As shown in FIG. 89, The ideal frame deflection occurs as a rotation around the Z-axis. The wave or corrugated sections allows for rotation of the Z-axis and reduces the twist caused by the rotation of the X-axis. As shown in FIG. 92A, a non-corrugated portion 462 is positioned between the corrugated portion 460 and the upper headgear strap securing means 432 in which the non-corrugated portion 462 of a resiliently stiff material may be adapted to reduce twisting of the frame arm 402. In another preferred embodiment, as shown in FIG. 92B, the first frame arm 402 or the second frame arm 420 may be a solid frame without any wave or corrugated geometry 464. In another preferred embodiment, as shown in FIG. 92C, the first upper frame arm 416 or the second upper frame arm 424 may each have a first wave geometry 466 in one direction 468 along the longitudinal axis of the upper frame arm 416/424, while the first lower frame arm 418 or the second lower frame arm 426 may have a second wave geometry 470 in a second direction 472 along the longitudinal axis of the lower frame arm 418/426. This rotated wave geometry may be preferred to match the complex surface of the face of the user such that the frame arm can bend in at the start and the upper or lower arms can bend up and in against the face of the user. In another preferred embodiment, as shown in FIG. 92D, the first upper frame arm or the second upper frame arm may have a blended wave geometry similar to the embodiment as shown in FIG. 92C but with a thicker border 415 around the wave geometry on the upper frame arm for reducing the effect of torsion on the frame arm, which may allow to maintain flexion against the face as well as to reduce or eliminate torsion of the first upper frame arm or the second upper frame arm.

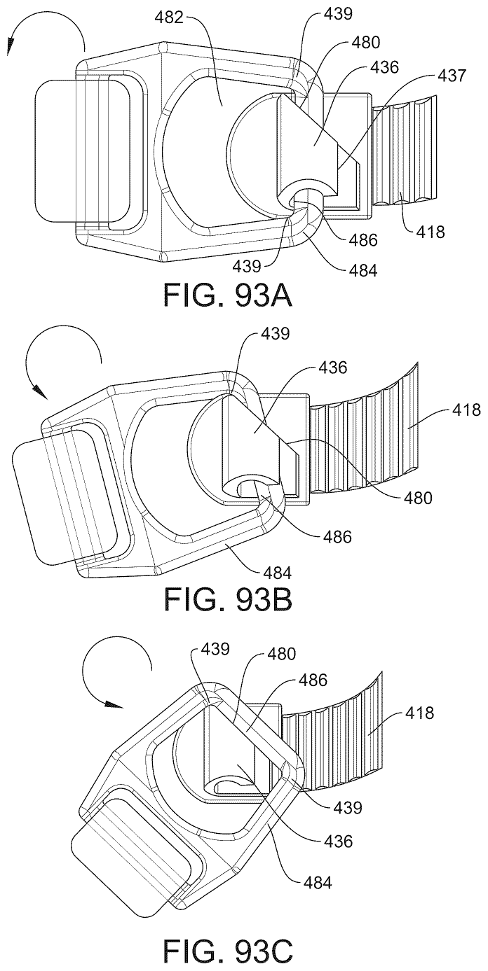

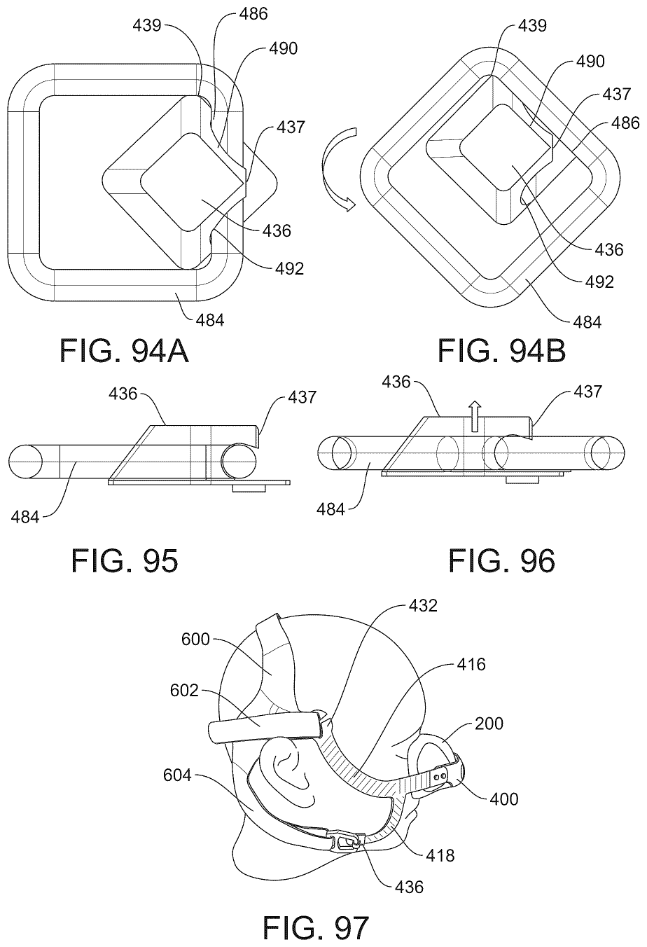

In another preferred embodiment, as shown in FIG. 93A, a hook 438 may be used at the end of the first lower frame arm 418, in which the hook 438 has a bevelled edge 480, which is illustrated to connect and secure to a loop portion 480 of a connector 484 attached to the lower headgear strap. FIGS. 93B and 93C shows by pivotally rotating the secured loop portion of the connector 484 such that the loop portion edge 486 is parallel with the bevelled edge of the bevelled hook, the connector 484 can allow for disengagement with the bevelled hook 436. While the tension of the headgear 600 when worn may pull and secure the connector 484 to the bevelled hook 436, and while possible for the user to push the connector 484 in the longitudinal direction such that the hook disengages from the loop portion edge 486 is no longer secured by the hook portion 437 of the bevelled hook 436, under high tension, it may cause injury to the user wearing the headgear 600 to apply a pushing force against the pulling tension. In another preferred embodiment, as shown in FIGS. 94A, 94B, 95, and 96, the hook 436 may be used at the end of the lower frame arm 418, in which the hook 436 has a first bevelled edge 490 in one direction and a second bevelled edge 492 in a second direction, showing that the pivotal rotation of a secured loop portion 484 of the lower headgear connector at a curved portion 439 either clockwise to be parallel with the first bevelled edge of the bevelled hook or anti-clockwise to be parallel with the second bevelled edge of the bevelled hook can each be allowed for disengagement of the loop portion 484 of the connector from the double bevelled hook 436.

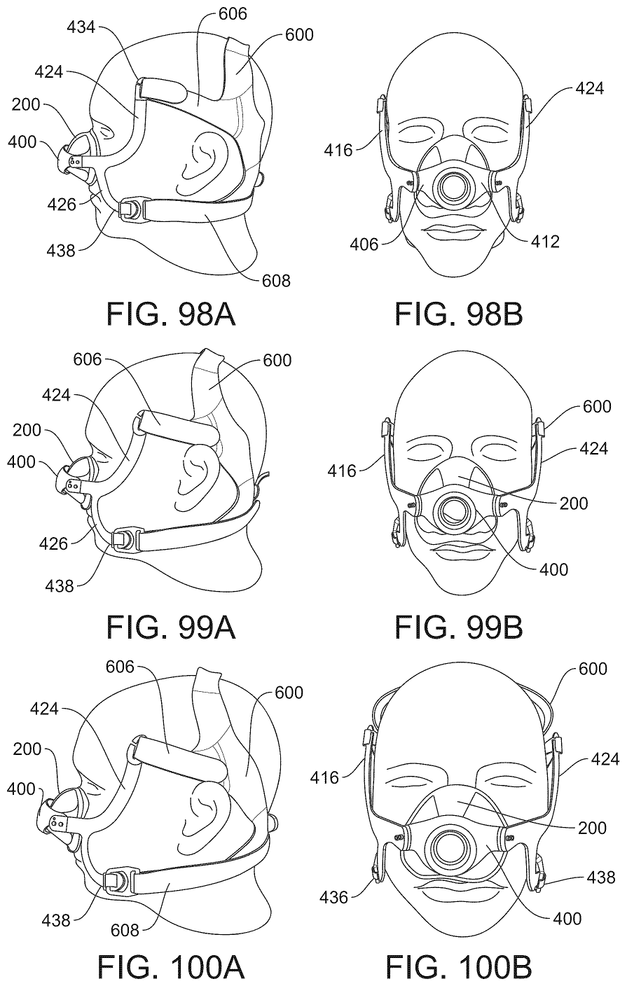

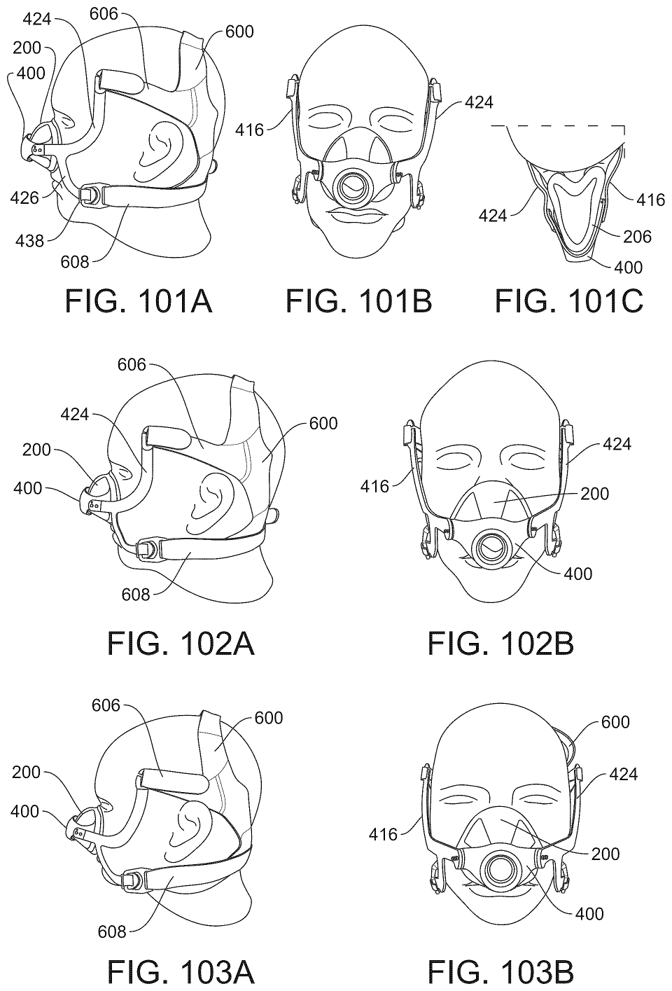

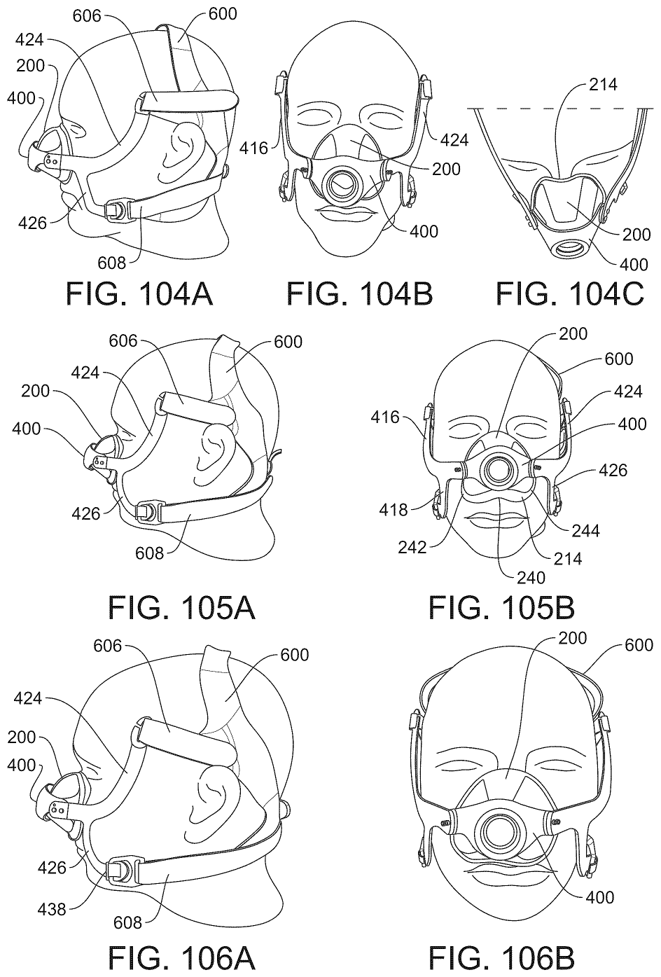

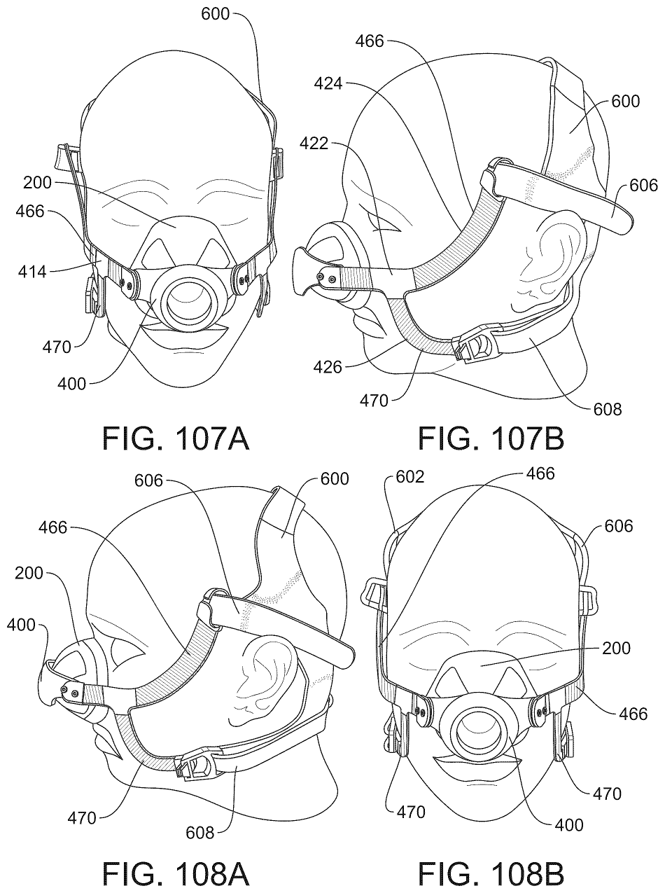

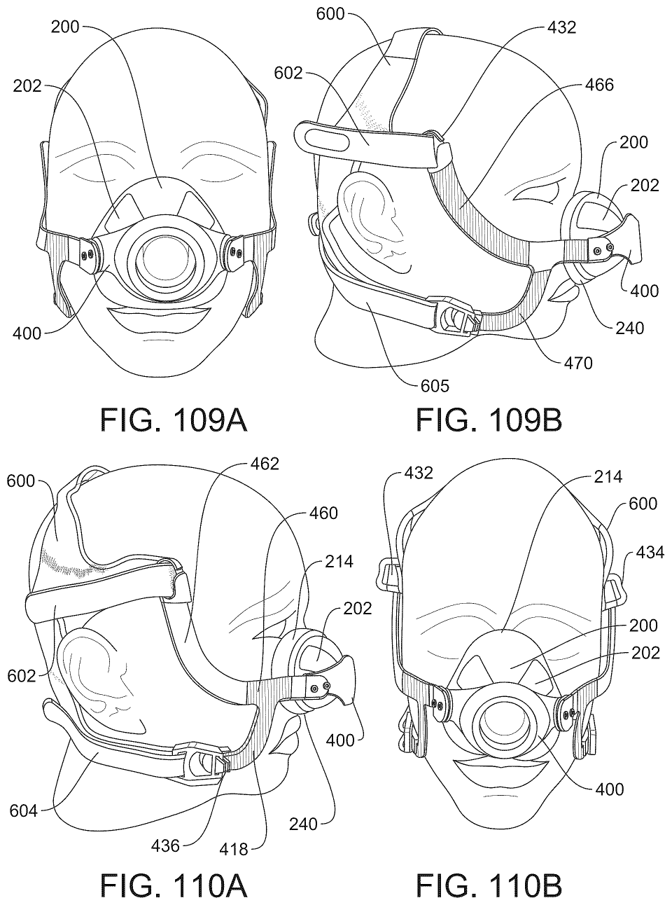

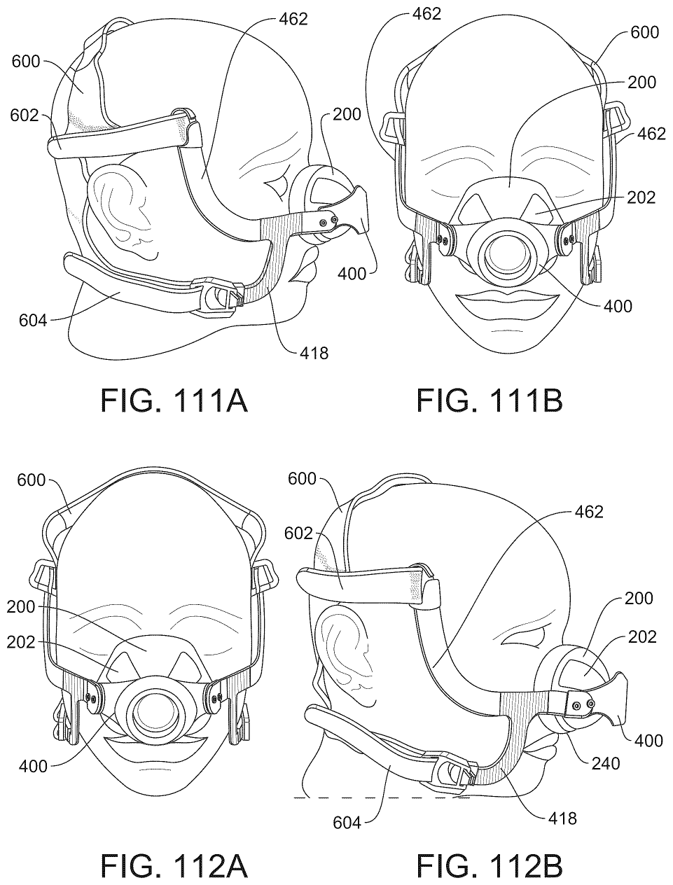

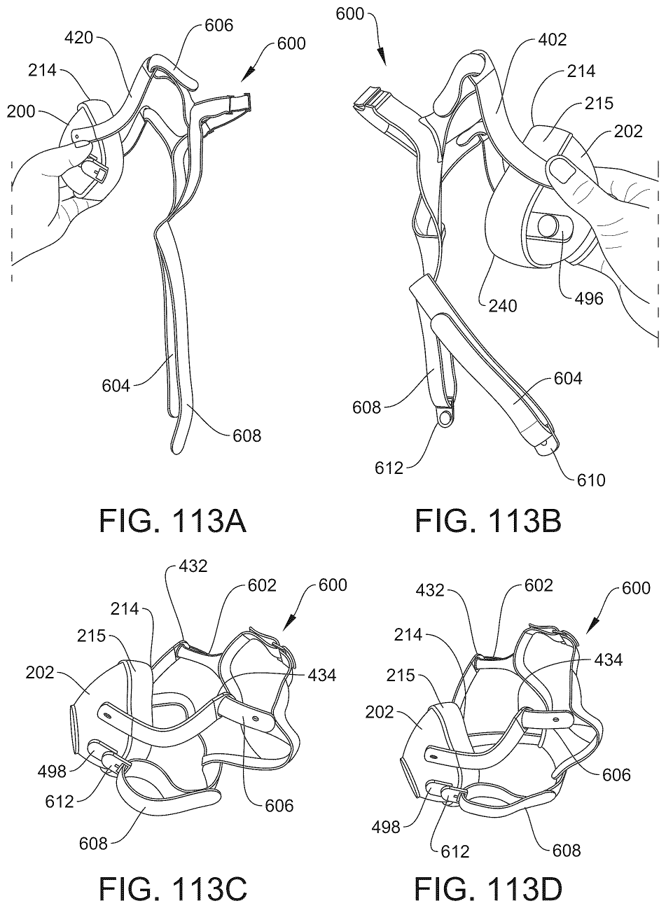

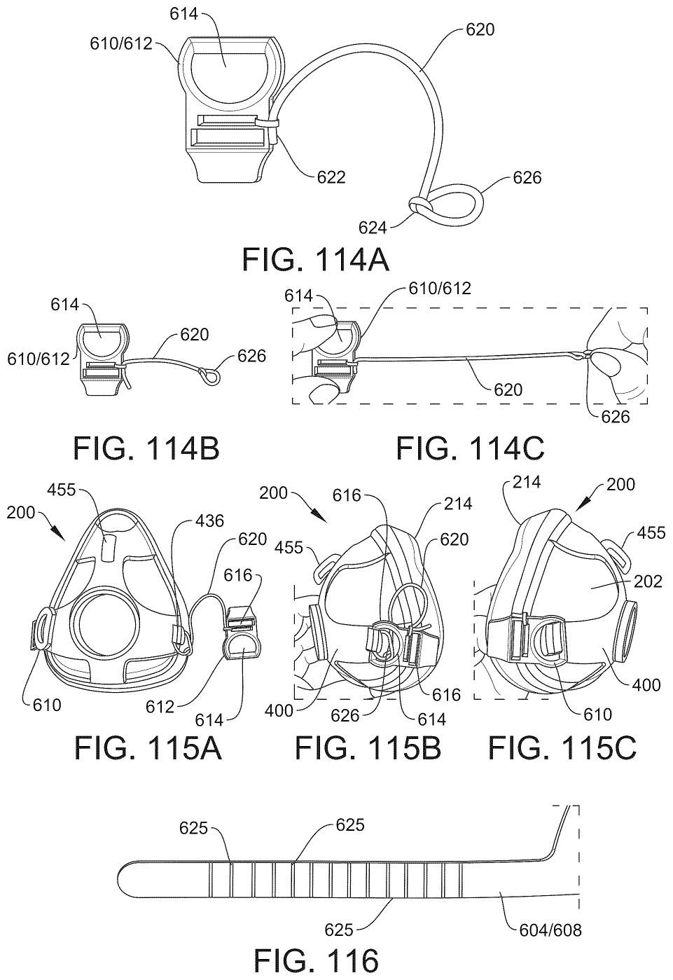

As it may be appreciated that different users have different and complex facial geometries (such as tall and narrow, short and wide, or an ‘average’ face, as shown in FIGS. 97 to 112B, different wave geometries in the upper frame arm and shaped to be bent at the cheek and at the upper terminal of frame arm may be preferred in the curvature contact testing. The testing on 3D printed head models allows the fitment of the mask and the mask assembly and determine the direction of forces and stresses to the head model to optimising the parts to be used for certain types of faces. As shown in FIGS. 113A to 113D, another challenge to overcome is to design a headgear 600 with the upper headgear straps 602/606 connected to the first and second upper frame arm connectors 432/434 that when simulating the action of putting over a user's head, the first lower headgear strap 604 and the second lower headgear strap 608 may be tangled, inconveniently falling behind the head of the user which requires the user to search and/or reach behind to hold the lower/bottom straps 604/608 which may have fasteners 610/612 to fasten/mate to the first lower connector 496 and the second lower connector 498 positioned at the lower end of the exoskeleton. In a preferred embodiment, as shown in FIG. 114A to 114C, there may be a first elastic tether 620 securable to a first lower headgear connector 610 or clip 610 at one end 622 and an elastic loop portion 626 at the other end 624. The securing position of the tether 620 may be at a location between a loop portion 614 and a slot 616. The loop portion 614 also secures at with the first lower connector 496 and the second lower connector 498 positioned at the lower end of the exoskeleton; and the slot 616 is for securing a first or second lower headgear strap 604/608. The exoskeleton 400 may have a hook portion for receiving and securing the elastic loop portion and the clip. The advantage of this modification is that the incidence of interference between the elastic tether and clip is significantly reduced and it provides a 160% increase stretch length for example from 5 cm to 13 cm. In another preferred embodiment, as shown in FIG. 115A to 115C, a mask 200 having an exoskeleton 400 with a first lower exoskeleton connector 496, and a second lower exoskeleton connector 498. This exoskeleton 400 having a central upper hook 455 extending outwardly and curled out of the front of the mask.

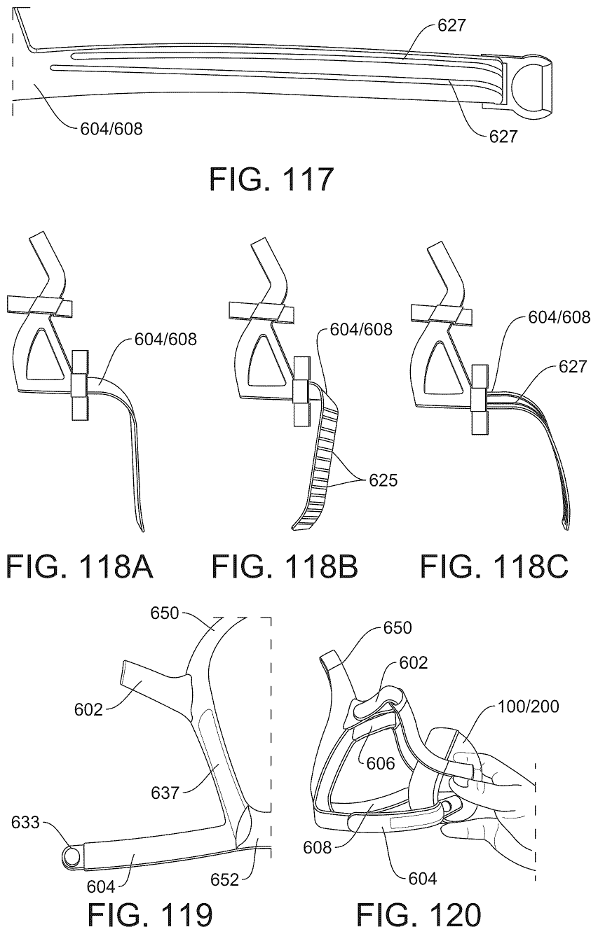

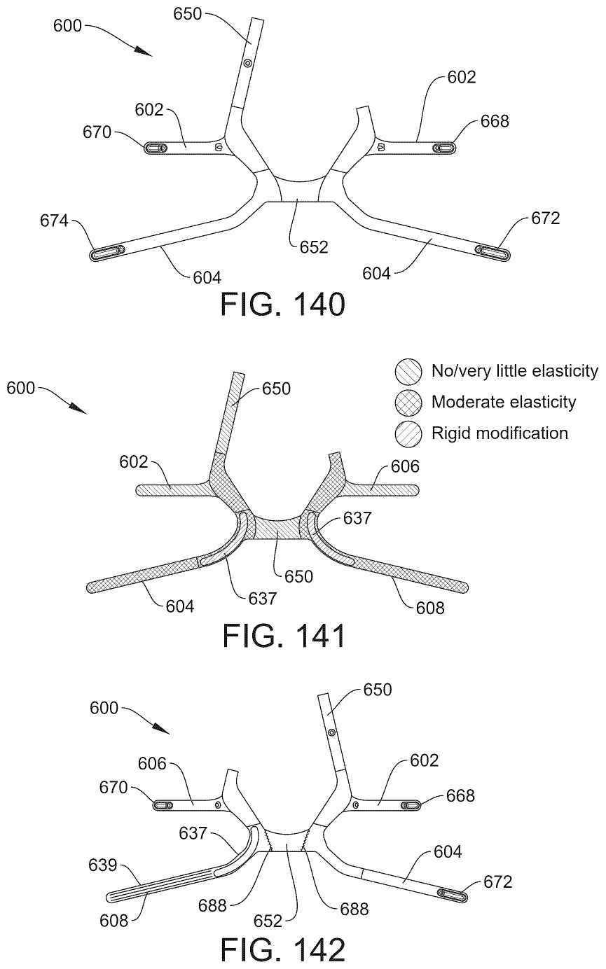

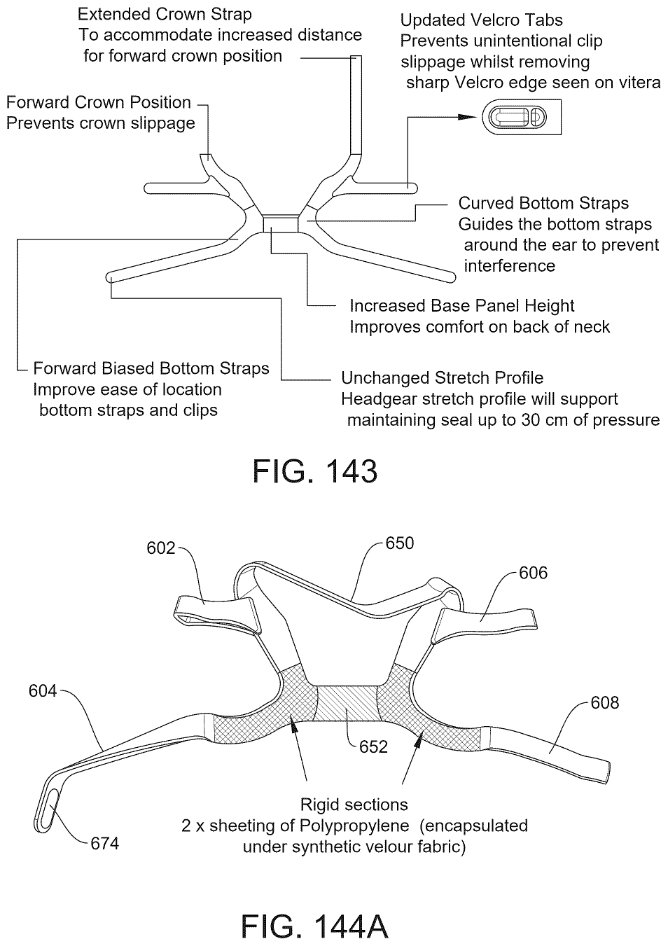

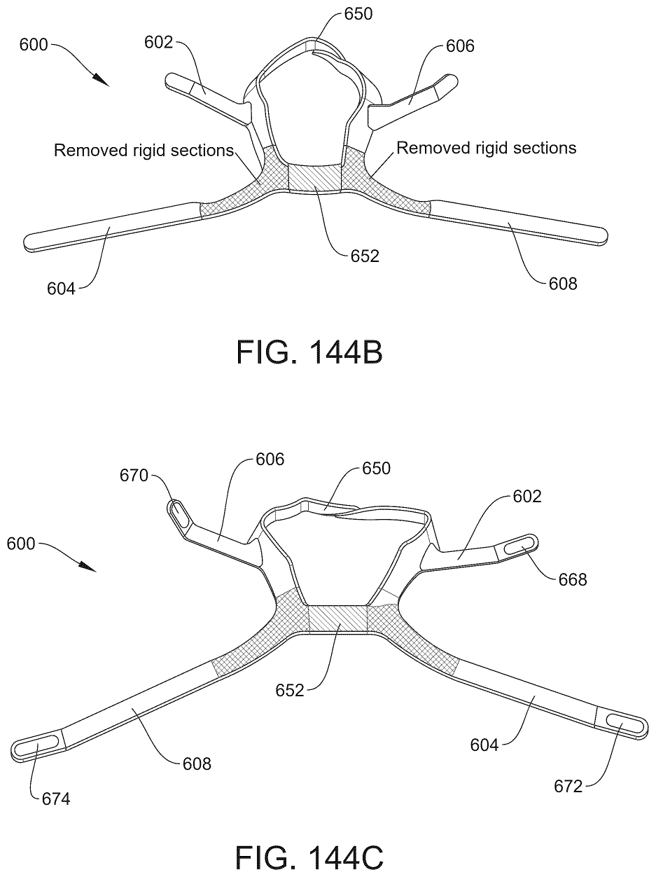

In another preferred embodiment, as shown in FIGS. 140 to 144C, the headgear 600 may comprise a variable a first structured lower headgear strap 604 and a second structured lower headgear strap 608, a first upper headgear strap 602 and a second upper headgear strap 606, a variable length crown strap 650 which can connect between the upper ends of the first strap and the second strap, and a base panel 652 which connects between the first structured lower headgear strap 604 and the second structured lower headgear strap 608. As shown in FIGS. 116 to 118C, the first lower headgear strap 604 and the second lower headgear strap 608 biasing are tested with the aim to increase the amount of the lower headgear straps or lower headgear elongate members to bias forward for user's ease in finding the lower headgear straps easily. The elongate members may have a plurality of perpendicular supports 625 along the longitudinal axis of the lower headgear straps 604/608. The perpendicular supports may be spaced a predetermined distance away from each other. For example, as shown in FIG. 116, the spacing may be 10 mm. It may be appreciated that different spacings can be used to provide a different droppage and twisting of the lower headgear straps 604/608. In another preferred embodiment, as shown in FIG. 117, there may be perpendicular supports 625 as well as one more linear support(s) 627. Strap biasing testing of the lower headgear straps 604/608 without perpendicular/linear supports (FIG. 118A) can be seen to drop and twist in this way and this default result can be used to compare whether the lower headgear strap with perpendicular supports only (FIG. 118B) and the lower headgear strap with the perpendicular and linear support (FIG. 118C) can perform better or reduce the drop of strap and/or twist. As the linear supports does provide an overall reduction in the drop of the strap, this is used to further improve the design.

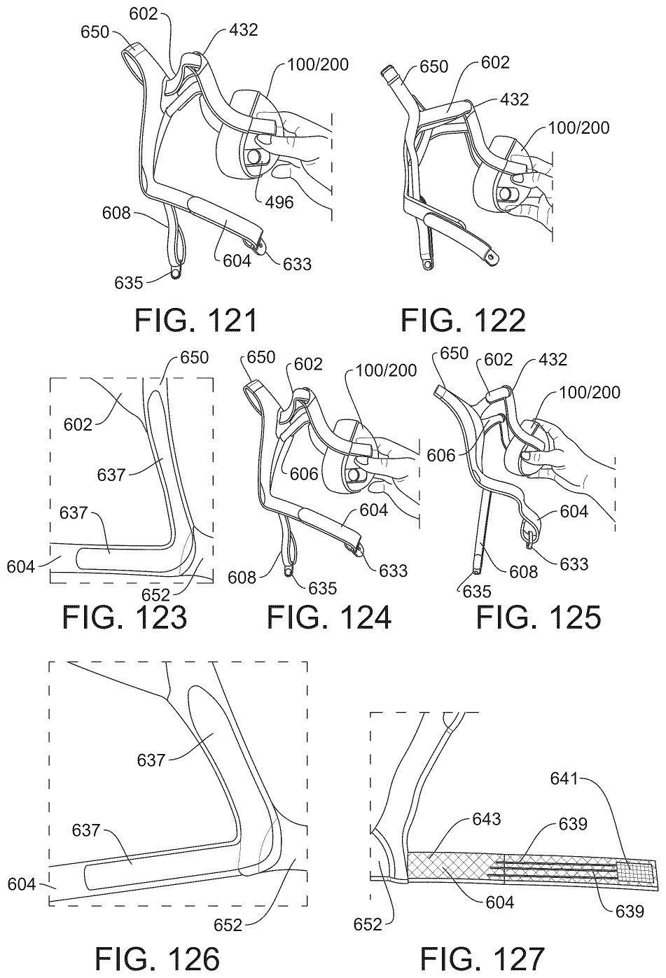

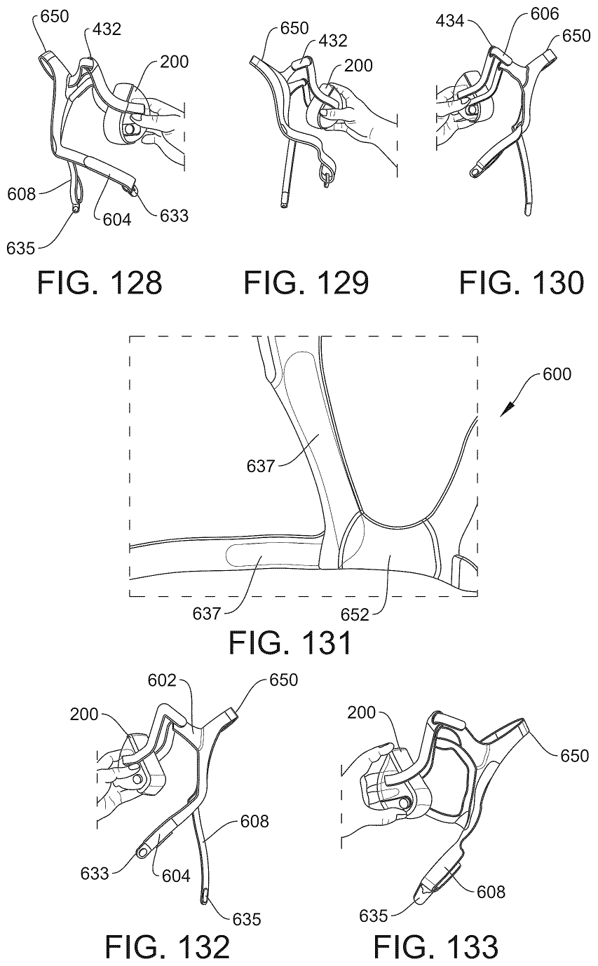

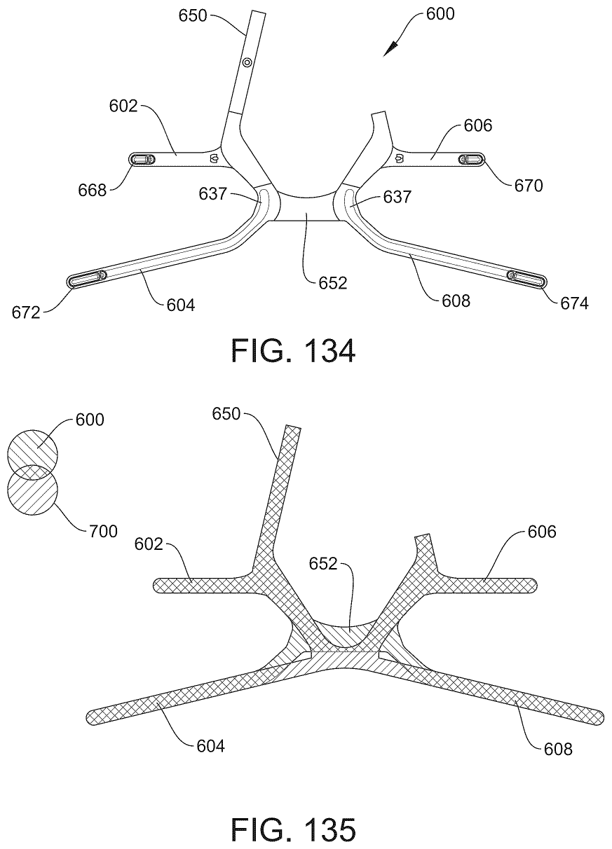

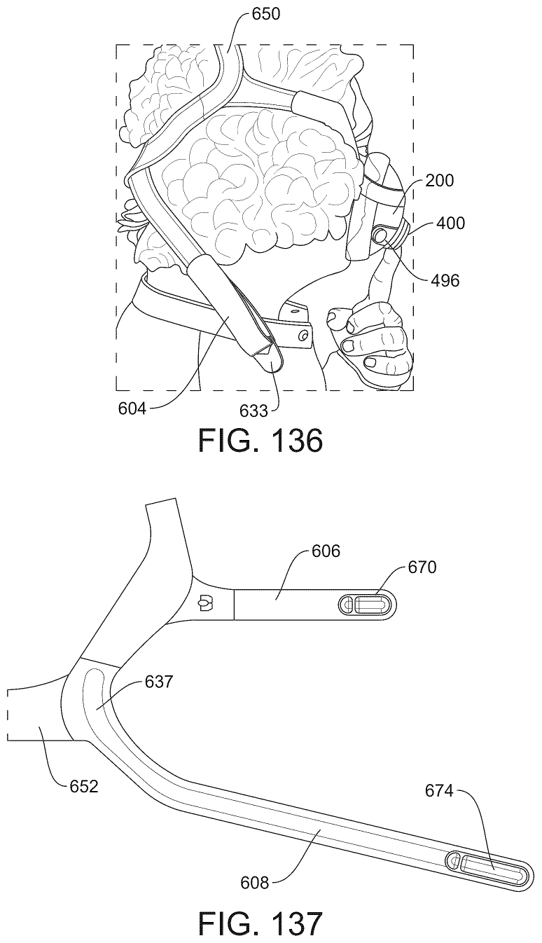

To assist the user in putting on the first lower headgear strap and the second lower headgear strap to the respective first lower exoskeleton connector and the second lower exoskeleton connector, as shown in FIG. 119, there may be a first quick connect tab 633 for the first lower headgear strap 604, and there may be a second quick connect tab 635 for the second lower headgear strap 608; and FIG. 120 shows the quick connect tabs 633/635 engaged in the respective lower exoskeleton connector 496/498 showing the mask assembly with the headgear attached. This embodiment as shown in FIGS. 120 and 123 has at least one layer of C-shaped sheet plastic 637 for providing support and structure to the lower headgear strap 604/608. This eliminates the need for added support. As shown in FIGS. 121, 122, 124, 125, the lower headgear straps 604/608 are biased to the mask 100/200 when held. FIG. 126 shows a front zoomed-in view partially of the headgear 600 with the lower headgear support layer 637 and FIG. 127 shows a back zoomed-in view partially of the headgear 600 with the lower headgear support layer with the linear supporting members 639 along the longitudinal axis of the elongate lower headgear strap positioned between a Velcro® hook fastener 641 and Velcro® loop fastener 643. As shown in FIG. 140, the headgear may comprise a custom crown strap that can be variable in length to accommodate increased distance for forward crown position, a forward crown position segment for preventing crown slippage, a soft interior that it made of a sustainable or hygienic material which can also have a premium feel, a touchpoint indicating section, a forward biased bottom headgear straps for improving the ease of locating bottom straps and clips; a curved bottom strap between the lower and upper head straps for guiding the bottom headgear straps around the ear to prevent interference, updated Velcro® tabs for preventing clip slippage whilst removing sharp Velcro® edges.

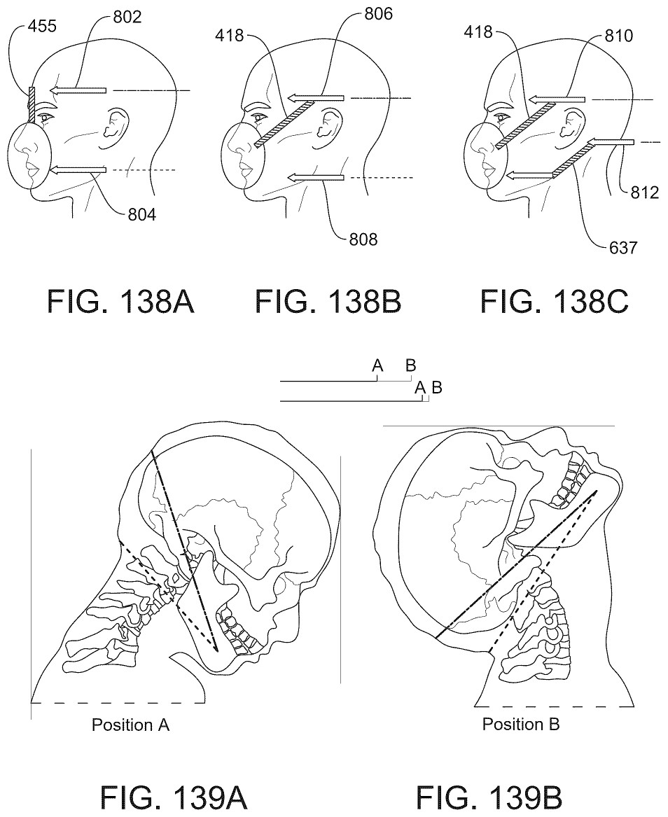

In showing the importance of designing the exoskeleton with frame arms and headgear for user comfort, FIG. 138A shows that with a forehead support 455, the force vector at the top of the head 802 is relatively comfortable, however, as there is less support at the bottom of the head 804, this region is uncomfortable for the user. FIG. 138B also shows that with an upper head support of using frame arms 418, the force vector at the top of the head 806 is relatively comfortable for the user, however, as there is less support at the bottom of the head 804, this region is uncomfortable for the user. FIG. 138C shows that with an upper head support of using frame arms 418, the force vector at the top of the head 810 is relatively comfortable for the user, and when there is also a structured bottom strap 637 used for the lower headgear straps 604/608 the bottom of the head is also well supported and is also relatively comfortable for the user. As shown in FIGS. 139A to 139B, knowing that a person's head can move their heads down and (Position A) and up (Position B), strap tension during movement is also tested as it can cause discomfort. It is found that there is less required strap extension when the point of distance is originating from the back of the skull.

As shown in FIGS. 140 to 144C, the headgear may further comprise an updated base panel 652 of a breathable material joining between the first curved bottom straps 604 and the second curved bottom straps 608. The joining may be a zig zag stitch 688 which can create stretchable seams without breaking. Further, when sewing fabrics that fray easily or have raw edges, zigzag stitches 688 can be used to secure and finish the edges, preventing them from unravelling. It also advantageously creates a clean, finished look. Keeping the force vectors in mind as well as taking into account the need to forward bias the lower straps, FIG. 141 shows the regions of the headgear which requires no or very little elasticity, regions of moderate elasticity, and regions of rigid modification.

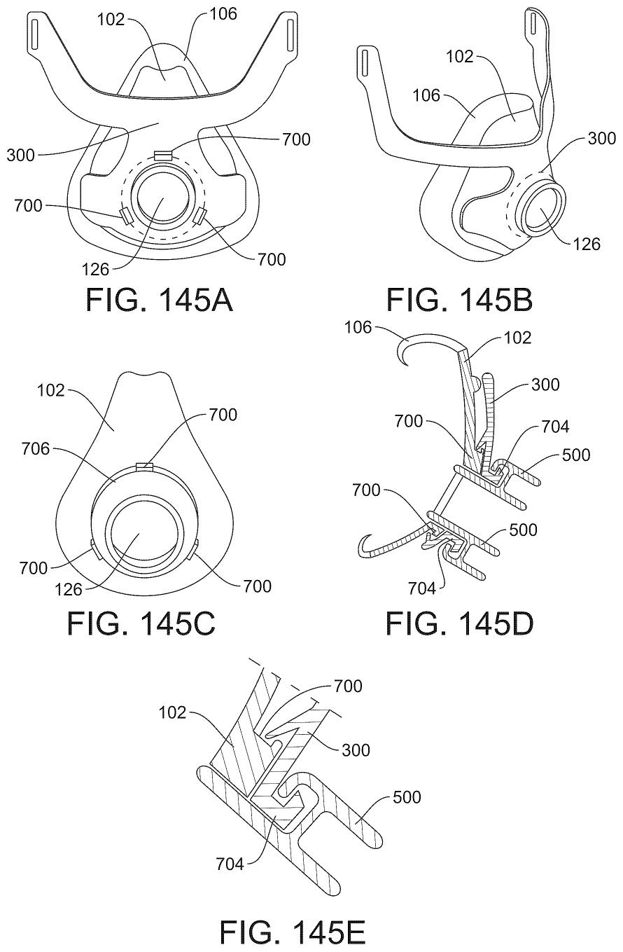

As shown in FIGS. 145A and 145B, in one preferable embodiment, there is a frame 300 mountable to an endoskeleton-cushion module 100. How the frame 300 is mountable or secured to the endoskeleton-cushion module 100 is shown in FIGS. 145C to 145E. As shown in FIG. 145C, the endoskeleton-cushion module 100 has an aperture 126 or bore 126 that is formed in shape of a non-circular snap or a tear-drop shape so as to advantageously ensures alignment of the frame 100 or exoskeleton 300 to the endoskeleton-cushion module 200. To assist in spreading the force from the connection of the frame 300 and the endoskeleton-cushion module 100, there may be one or more retaining portions 700 for snapping or interlocking with frame 300. As depicted in FIG. 145A, an example may be three frame retention means 700 equidistantly spaced around the perimeter 706 of the non-circular shape. It may be appreciated that for a more secure embodiment, it may be appreciated that there can be more frame retention means 700 that can be put around the perimeter of the aperture 126 of the endoskeleton-cushion module 200. In this preferred embodiment, as shown in FIGS. 145D and 145E, the endoskeleton-cushion module 200 may have a frame interlocking portion 700 for receiving and securing the back of the frame 300, in which the front of the frame 300 may have an elbow interlocking portion 704 for receiving and securing an elbow portion 500. In this embodiment, this configuration may be advantageous in that when assembling, the elbow 500 cannot be secured to the endoskeleton-cushion 200 without having a secured frame 300 to the mask. An elbow 500 or joint connecting to a positive air pressure hose to the mask frame 300 can advantageously allow for greater flexibility, as wearers can move more freely during sleep, as they roll to the side or when sleeping in a supine position, without dislodging the mask or creating leaks. This ability to move without interrupting the therapy may enhance sleep quality and may allow a wearer to adhere to wearing the mask for therapy. The added mobility may reduce the pull on the endoskeleton-cushion module 100 from the hose, which can be particularly beneficial for side sleepers or those who tend to move a lot in their sleep. This preferred embodiment for interlocking the elbow to the frame 300 can decrease pressure masks and discomfort to the face, which can lead to a more comfortable night's sleep.

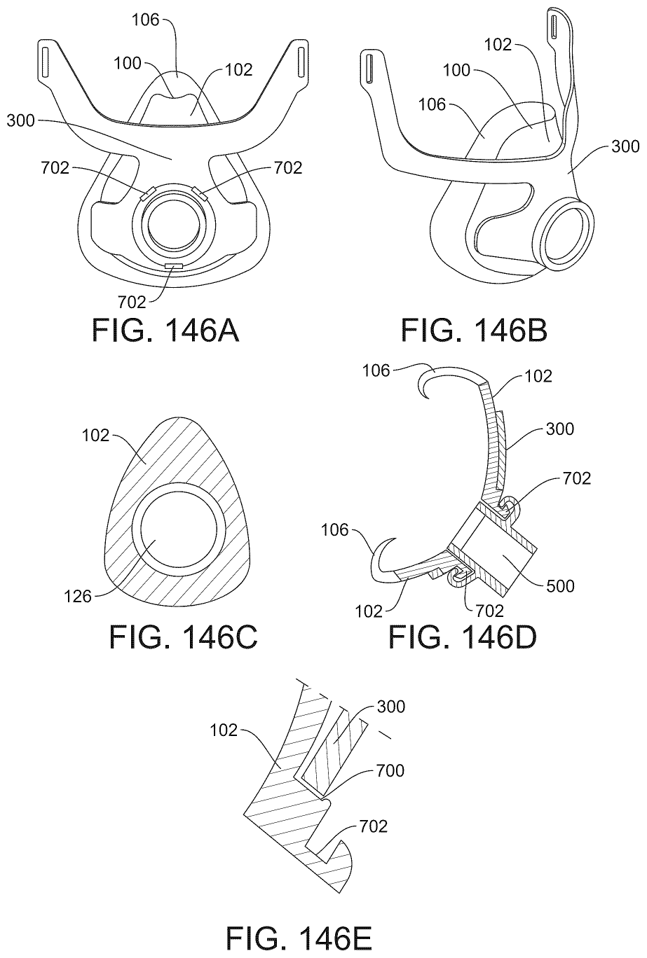

As shown in FIGS. 146A and 146B, in another preferred embodiment, there is a frame 300 mountable to an endoskeleton-cushion module 100. How the frame 300 is mountable or secured to the endoskeleton-cushion module 100 is shown in FIGS. 146C to 146E. As shown in FIG. 146C, the endoskeleton-cushion module 100 may also have an aperture 126 or bore 126 that is formed in shape of a non-circular snap or a tear-drop shape so as to advantageously ensures alignment of the frame 300 or exoskeleton 300 to the endoskeleton-cushion module 100. To assist in spreading the force from the connection of the frame 300 and the endoskeleton-cushion module 100, there may be one or more frame retaining portions 700 for snapping or interlocking with frame. As depicted in FIG. 146A, an example may be three frame retention means 700 equidistantly spaced around the perimeter of the non-circular shape. It may be appreciated that for a more secure embodiment, it may be appreciated that there can be more frame retention means 700 that can be put around the perimeter of the aperture portion of the endoskeleton-cushion module 100. In this preferred embodiment, as shown in FIGS. 146D and 146E, the endoskeleton-cushion module 100 may have a frame interlocking portion 700 for receiving and securing the back of the frame 300. At a separate location, the endoskeleton-cushion module 100 may have an elbow interlocking portion 702 for receiving and securing the elbow 500. In this preferred embodiment, this configuration may allow the resilient endoskeleton 102 to absorb and distribute the force when attached to the elbow 500. The elbow 500 cannot be secured to the endoskeleton-cushion 100 without having a secured frame 300 to the mask. The advantages of this preferred embodiment may have improved seal stability in that attaching the elbow 500 to the endoskeleton-cushion module 200 can help stabilise the mask on the wearer's face. Since the resilient endoskeleton 102 may provide a relatively rigid/semi-rigid structure to the mask, any movement of the hose connected to the elbow 500 from the wearer's sleeping position are less likely to disturb the seal between the cushion and the face. This embodiment can significantly reduce air leaks, which can be an innovative solution to a common problem that can undermine the effectiveness of CPAP therapy. Furthermore, the advantage of this preferred embodiment is that it can lead to a better distribution of pressure across the mask and thereby enhancing the comfort of the wearer. This streamlined design can result in more compact and streamlined mask design can be particularly beneficial for wearers who may feel claustrophobic with larger masks of those who may prefer a minimalistic design that obstructs their view less, making activities like reading or watching TV before sleep more accessible. Since the elbow's movement is closely integratable with the mask structure, it can better adapt to changes in sleeping positions without causing leaks or discomfort. This adaptability can be extremely beneficial for wearers who may move frequently during sleep but still require consistent therapy.

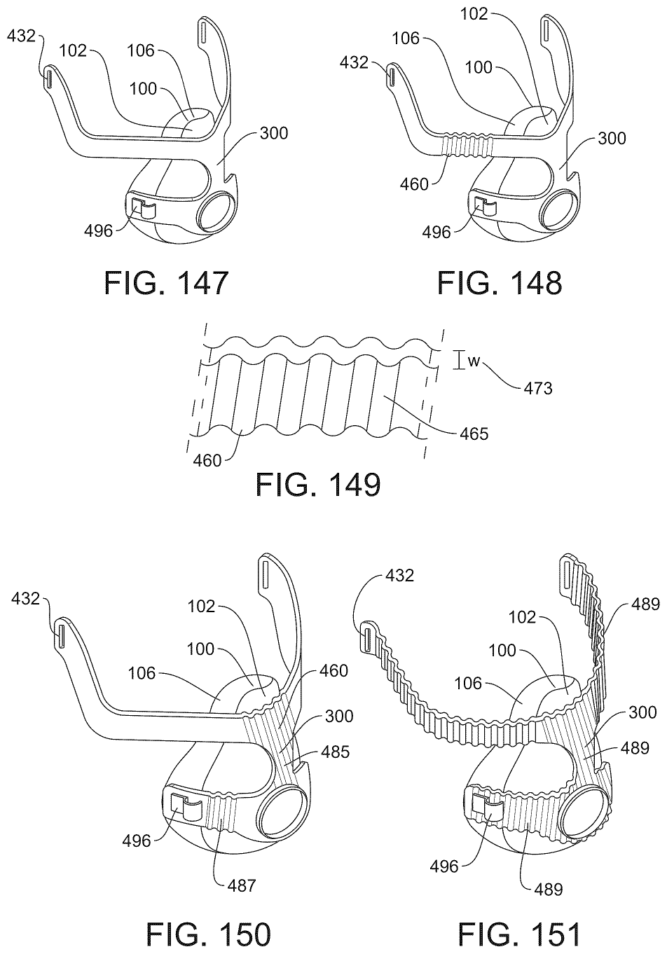

As shown in FIG. 147, in one traditional embodiment for the frame 300, a frame 300 without corrugations is shown. As shown in FIG. 148, a preferable embodiment for the frame or exoskeleton 300 is shown to have a corrugation portion 460 along a portion of the upper frame. For simplicity in depicting the positioning of the corrugation portion 460, it may be appreciated that that a similar positioning of the corrugation portion 460 is also on the other side of the upper frame (not shown) such that it is symmetrical. The corrugated portions may offer an advantage to provide flexibility and strength to a certain or specific part of the upper frame when needed. This embodiment of the frame can distribute and direct forces, allowing it to adjust and fit a wider variety of facial contours.

As shown in FIG. 149, the advantages provided by the corrugation portion 460 are: that it can be able maintain stiffness whilst allowing flexibility in desired directions, allowing the frame 300 to conform around the wearer's face, improves the ability of the mask system, improving over a hinge as uniform wall corrugation thickness can give flexibility without weakness or weak point, and the uniform corrugation profile can enhance the flow of the mold process. For example, with plastic injection moulding processes, the consistent wall section will produce more consistent moulding properties, with less in-mould stresses, and better melt flow thus producing a higher quality and durable component, which is less prone to failure in use. As shown in FIG. 150, a corrugated portion 460 may be positioned along the centre portion 485 and a partial corrugated frame portion 487 may be positioned along the lower arms of the frame or exoskeleton. The centre corrugation portion 485 can offer better shock absorption and thereby distributing impact forces more evenly across the frame 300. This can protect the frame 300 and its wearer from potential damage or injury resulting from bumps, drops, or external pressures. As shown in FIG. 151, another preferred embodiment of the frame 300 is that the frame can be corrugated all over 489. This preferred embodiment may advantageously provide better shock absorption as well as increasing the strength and durability of the frame, where the corrugation can provide an increase to the structural integrity of the resilient material, allowing it to withstand more stress and pressure without deforming. Beyond functional benefits as described, corrugated designs can also offer unique aesthetic options, thus allowing for distinctive looks that can be both visually appealing and recognisable. As it can be visually appealing to wearers, it also can advantageously impact on the wearer's compliance as they may more likely want to wear during therapy compared to a product that is not visually appealing.

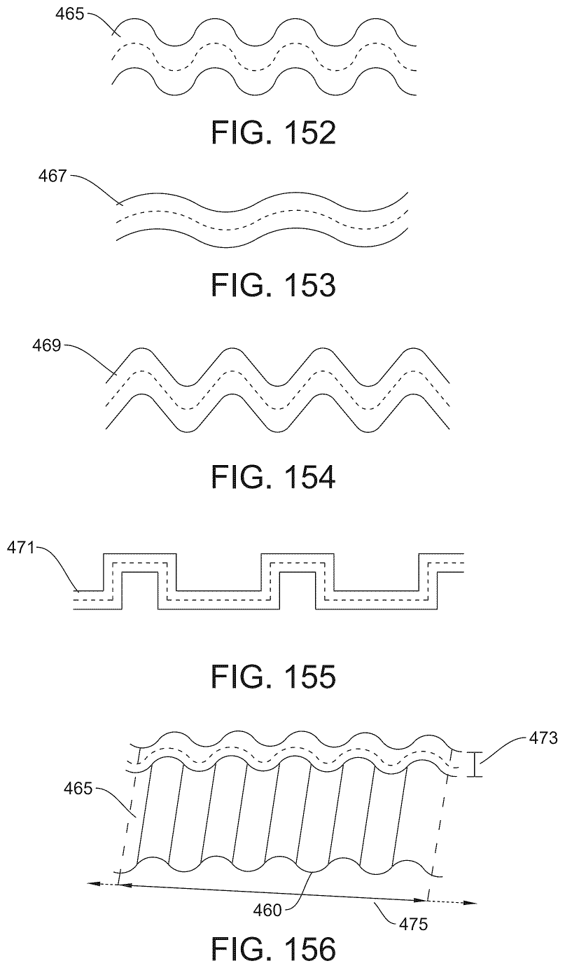

As shown in FIGS. 152 to 155, different embodiments to the corrugation profiles are shown, which in itself offers different flexibility characteristics which may be beneficial to certain face shapes.

FIG. 152 shows a schematic view of a sinusoidal 465 or circular profile corrugation 465 for use of a corrugated frame portion 460. Sinusoidal corrugations 465 can provide a high strength-to-weight ratio, which adds rigidity and strength without significantly increasing the weight of the frame, making it ideal for applications where lightweight structures are desired. Further, the advantage of using a sinusoidal corrugation profile 465 can be effective in absorbing the ‘pistoning’ effect as pressure swings in the mask occur during breathing or when using bi-level therapy. The sinusoidal corrugations will advantageously absorb this movement better. Furthermore, additional benefits are that it potentially provides controlled elongation when headgear straps are overtightened thereby not transferring excessive headgear tension loads onto the face of the wearer. This can lead to a reduction in noise and an increase in the lifespan of the frame and any attached components by minimising fatigue stresses. The increased structural integrity and flexibility is useful as it the sinusoidal pattern allows for greater flexibility in certain directions, depending on the orientation of the corrugations. This flexibility can be particularly beneficial in applications where the frame needs to accommodate movements or adjust to varying loads without compromising its structural integrity.

FIG. 153 shows a schematic view of an elliptical profile corrugation 467 for use of a corrugated frame portion 460. Using an elliptical corrugation profile 467 for a frame 300 can advantageously introduce a unique specific structural property to the frame. The targeted strength distribution can allow for more precise control over where strength is added within the frame. By adjusting the orientation and aspect ratio of the ellipses, the corrugation can be tailored to have increased rigidity in desired directions more suitable for a wearer's unique face shape.

FIG. 154 shows a schematic view of a triangular profile corrugation 469 for use of a corrugated frame portion 460. This geometric configuration of triangular corrugations 469 is inherently strong, offering superior resistance to bending and compression compared to other corrugation profiles as well as efficient load distribution, in which the angular nature of triangular corrugations 469 facilitates more effective distribution of forces throughout the structure. This can help in evenly dispersing stress, reducing the likelihood of localised failure points, and enhancing the overall durability of the frame.

FIG. 155 shows a schematic view of a square 471 or rectangular profile corrugation 471 for use of a corrugated frame portion 300. The simple geometry of square/rectangular corrugations 471 can make it relatively easy and cost-effective to manufacture. Rectangular corrugations 471 can provide a high degree of structural integrity and stiffness, and that the straight lines and angles of the rectangular or square profile distribute weight and stress efficiently, thereby contributing to the overall strength of the structure.

FIG. 156 illustrates a schematic representative corrugation where there is consistent wall section width 473 from any of the corrugation profile types, and that the corrugated portion 460 is made from a resilient material allowing elongation under headgear tension. The corrugated portion 460 is made from a resilient material allowing elongation under excessive headgear tension, and thereby improving the fitting as well as improving the stability during side sleeping. It may be appreciated that varying wall section corrugation widths, for example, from relatively thicker to relatively thin from left to right or right to left along at least a portion of the exoskeleton or frame 300 may advantageously allow a better customised fit to better conform to different face shapes and sizes. It may also be appreciated that besides the thickness or width that can be varied to obtain the right flexibility in certain zones of the face shape, the height of the corrugations can also be varied for optimising the stiffness and in part, the flexibility of the corrugated portion of the exoskeleton or frame where desired force load transfers and customised fit can be optimised at the right zones of the wearer's face shape.

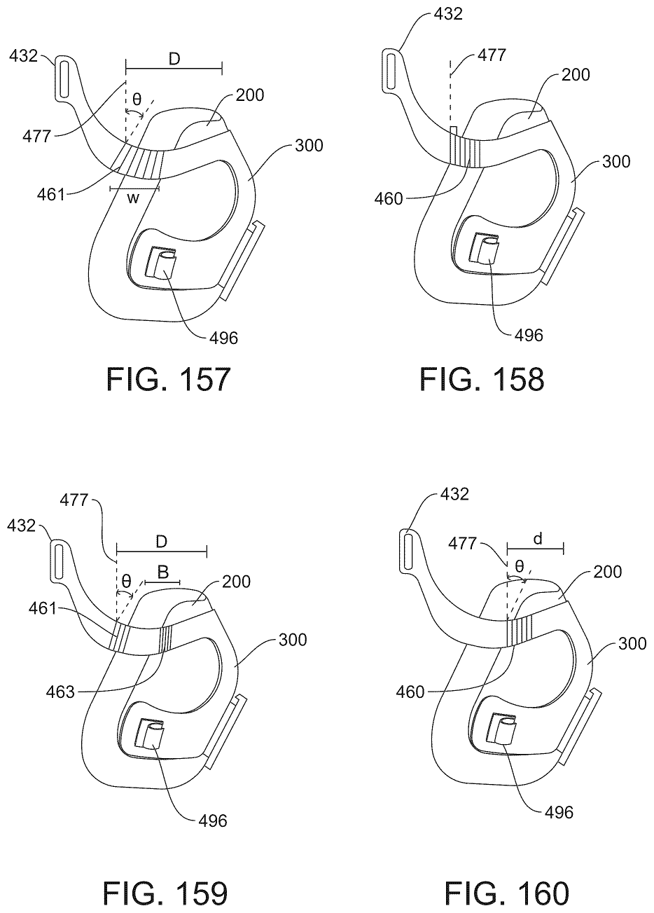

FIG. 157 illustrates a side view of another preferred embodiment of the frame mounted to the endoskeleton-cushion module 100, in which the corrugations 460 may be diverted at an angle θ° relative to the vertical axis 477 of the mask 100. The angled corrugations 461 can advantageously introduce a dynamic approach to structural design, and blending the benefits of corrugation with enhanced performance characteristics tailored or optimised for wearer's face shape. By angling the corrugations, the stiffness and strength of the frame can be significantly enhanced in desired directions. This directional reinforcement allows for tailored performance of the frame, and making the exoskeleton more resilient against bending, and twisting. Furthermore, the width between corrugations 479 may be varied for adjusting the flexibility of the frame 300.

FIG. 158 illustrates a side view of another preferred embodiment of the frame 300 mounted to the endoskeleton-cushion module 200, in which the corrugations 460 are parallel to the vertical axis 477 of the mask. It may be a shifted angle compared to the embodiment shown in FIG. 157. The shifted angle allows the corrugation with different angle (θ°) to change the direction of deflection, which is also optimised for face shape in certain situations.

In another preferred embodiment, as shown in FIG. 159, the upper frame may have multi-segments 461, 463 of corrugated portions 460, which are spaced apart relative to each other. The different segments 461, 462 may each be angled and allow bending at different points in the frame 300. This embodiment may be favourable for wearers where more flexibility is needed.

In another preferred embodiment, as shown in FIG. 160, the upper frame may have a corrugated portion 460 which is located anywhere along the upper frame. The corrugated portion may be shifted relative to another embodiment (shown in FIG. 158). The shifted position of the corrugated portion 460 may allow for desired routing of forces and directing stress pathways at a different location which may be more suitable for different wearer's face shape. By carefully routing forces, stresses can be evenly distributed across the frame, thereby minimising points of concentration that could lead to material fatigue and deformation. Further, this preferred embodiment also beneficially provides improved contouring around facial contours, and providing improved side sleeping performance because the corrugations remain more intimately in contact with the wearer's face such that when the mask is displaced sideways relative to the face, the corrugations are compliant enough to remain closely contoured and intimate to the face, which advantageously decreases contact with bedding material that can cause mask and cushion instability.

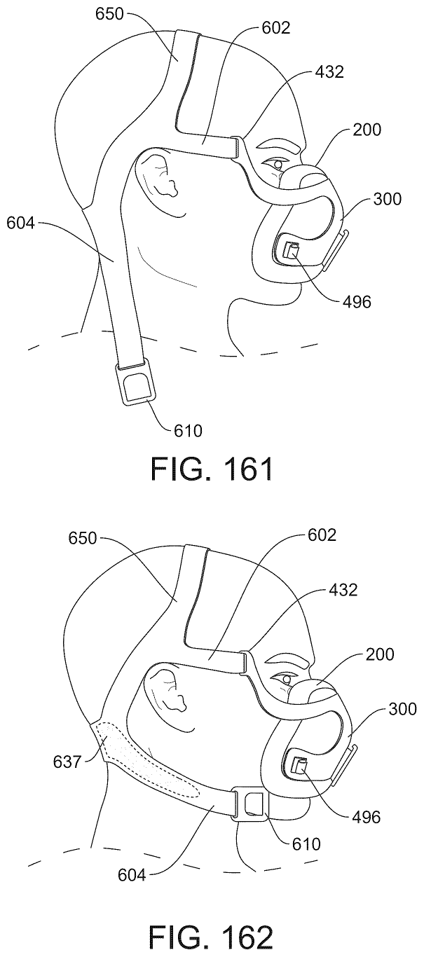

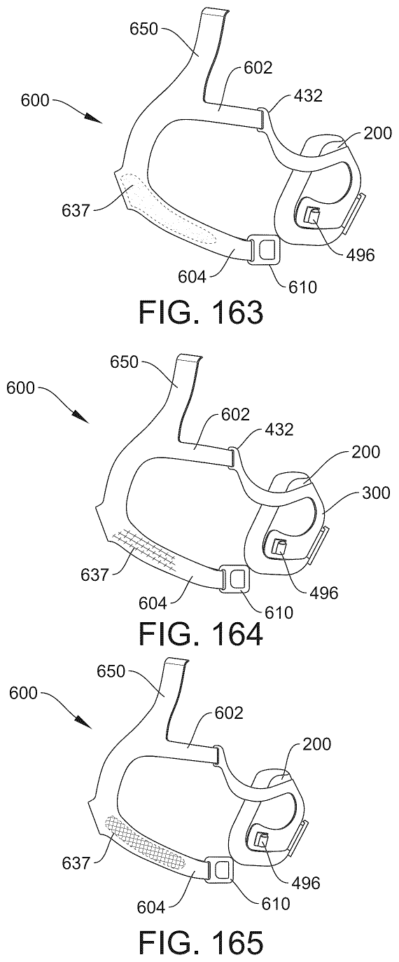

FIG. 161 shows a side view of a wearer wearing a frame 300 mounted to an endoskeleton-cushion module 200 with a traditional headgear with unbiased lower headgear straps 604. The headgear's upper strap 602 secured to the strap receiving means 462 located at the end of the upper arm of the frame 300. This figure shows that the unbiased lower headgear straps 604 falls behind the neck of the wearer and that the hook 610 for securing to the lower portion of the mask 496 is relatively far away from each other. As such, a wearer who may not be dextrous may find it extremely difficult to grasp the lower headgear strap 604 to hook 610 to the securing means 496 on the frame 300. Compared to the improved version as shown in FIG. 162, this figure shows a side view of a wearer wearing a frame 300 mounted to an endoskeleton-cushion module 100 with a headgear with a forwarding biasing lower headgear straps 604. The headgear's upper strap 602 secured to the strap receiving means 432 located at the end of the upper arm of the frame 300. This figure shows that the forward biasing lower headgear straps 604 falls forward or in front of the wearer's shoulder showing that the hook for securing to the lower portion of the mask is relatively closer to each other. It thereby allows a less dextrous wear to easily find the lower headgear strap 604 and thereby improves the usability for a person to hook onto the frame 300. FIGS. 163 to 165 show that by putting a stiffening element 637 such as a stiffener 637, or a stitched seam 637, or a Velcro 637 accessory along the lower headgear strap 604 at the indicated position can provide a forward bias of the lower headgear strap 604. As shown in FIG. 163, the resilient material may be made of metal or plastic or modified fabric, and it is not 100% rigid. The stiffener 637 may be an over moulded plastic, sewn stiffener, or a laminated stiffener. The stiffening portion 637 for use to the lower headgear strap 604 to bias forward, the stiffening portion 637 may be manufactured by heat staking materials to get to the desired stiffness, gluing materials together to get to the desired stiffness, and stitching to the lower headgear strap a stiffener, using a thicker fabric, stitched seams to provide more stiffness, using a thermoformed shape, and the adjustment of stiffness can be done through different length/tension on an inner fabric relative to the outer fabric.

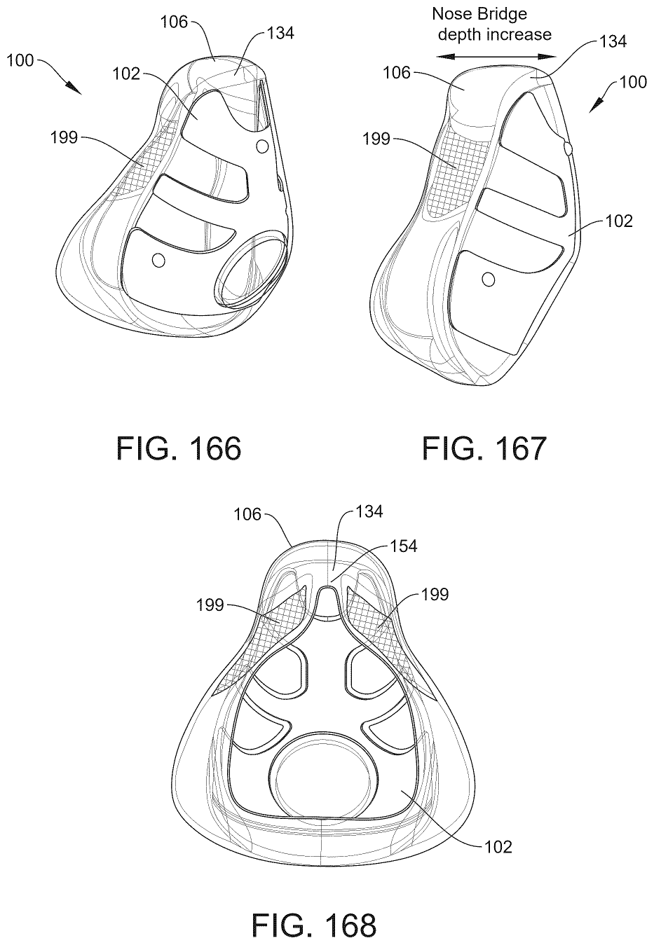

FIG. 166 illustrates a perspective view of the endoskeleton-cushion module of another preferred embodiment, showing the cushion rib at nose bridge contoured inwards to improve comfort. This preferred embodiment represents a deliberate design aimed at enhancing wearer comfort. The nose bridge is a common point of discomfort in respiratory masks and respirators due to pressure and the potential for chafing or irritation. By contouring the cushion rib inwards, it may advantageously achieve an improved pressure distribution. The inward contour may help to distribute the pressure exerted more evenly around the nose bridge, reducing certain sports of discomfort. This contoured design could also advantageously better accommodate the natural variations in nose bridge shapes and sizes for different people, leading to a more universal fit. By ensuring a snug and comfortable fit, the design can also prevent excessive air leakage around the nose bridge. FIG. 167 illustrates another perspective view of the endoskeleton-cushion module of FIG. 166 showing the spiral thickened side 199 of the nose section to optimise load distribution and compliance along nose contour for certain wearer face shapes. In this embodiment, there is an increase in nose bridge depth. The spiral design is an advantageous geometric or structural modification that incorporates a membrane of a spiral pattern which thickens at strategic points along the nose section of the cushion. It allows for a more adaptable fit that can flex and conform to the unique contours of the wearer's nose. This adaptability may ensure a tighter seal and greater comfort, especially for individuals whose facial features may not align with the average dimensions for which standard or conventional masks are designed. FIG. 168 illustrates a back view of the endoskeleton-cushion module of FIG. 167 showing that the thicker parts of silicone spiral highlighted allows transfer cushion force around side and contour of the nose. It is advantageous for parts of nose that easily changes geometry quite rapidly.

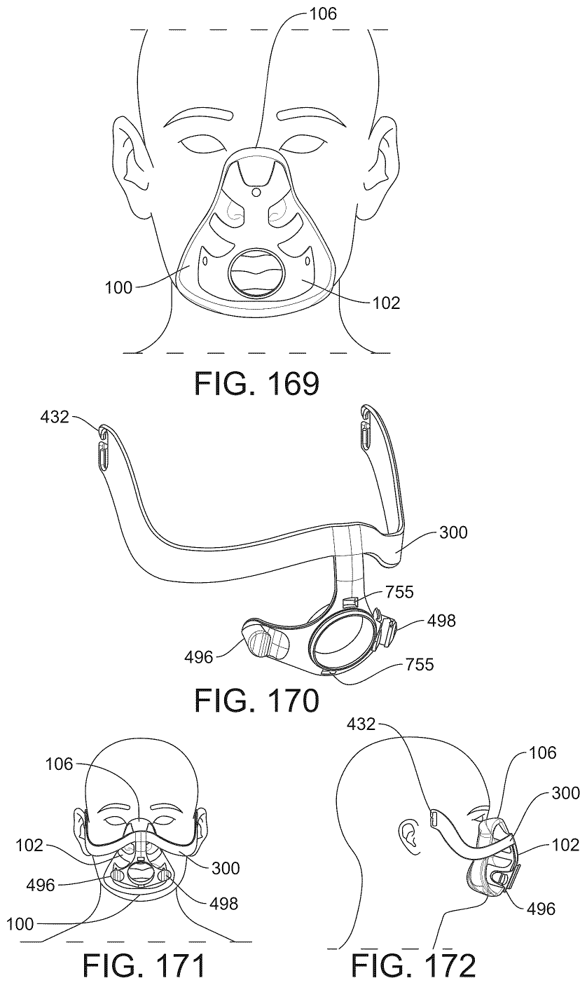

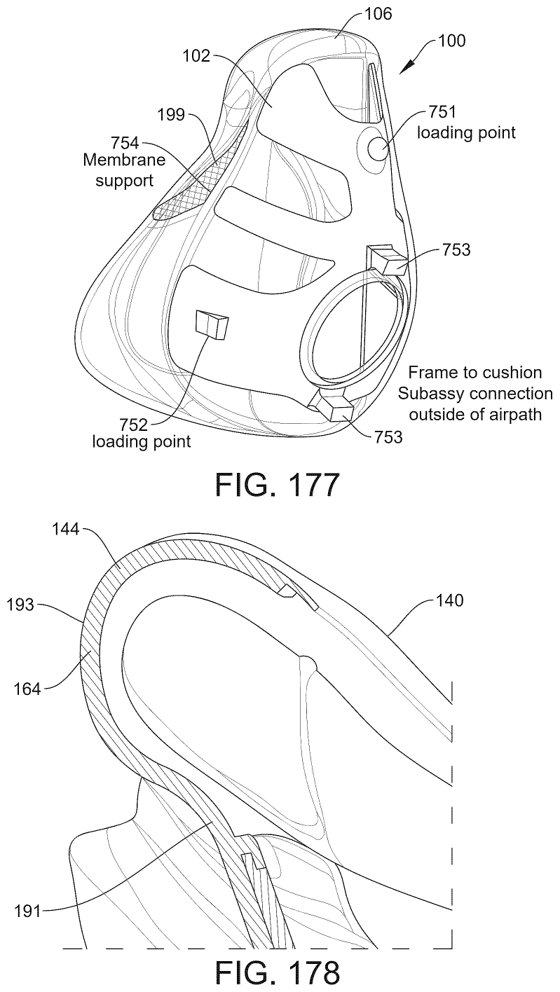

As shown in FIG. 169, FIG. 169 illustrates a front view of a wearer wearing the endoskeleton-cushion module of FIG. 168 showing a nice and snug fit to the face. FIGS. 170 and 171 illustrates a perspective and a side view of an exoskeleton or a frame of a preferred embodiment showing that the center of the frame is suitably shaped so as to transfer force to the endoskeleton 102 via contact points 751, 752 (left), 752 (right—not shown) on the endoskeleton 102 as shown in FIG. 177. In this preferred embodiment, the frame may have a retention snap or a frame to cushion subassembly connection outside of airpath to interlock with the endoskeleton-cushion module. There is also shown that the lower frame portion may have bumps to improve ease of removal of a hook tethered to the lower headgear strap. FIG. 171 illustrates a front view of a wearer wearing the frame of FIG. 170 that is secured to the front of the endoskeleton-cushion module of FIG. 169.

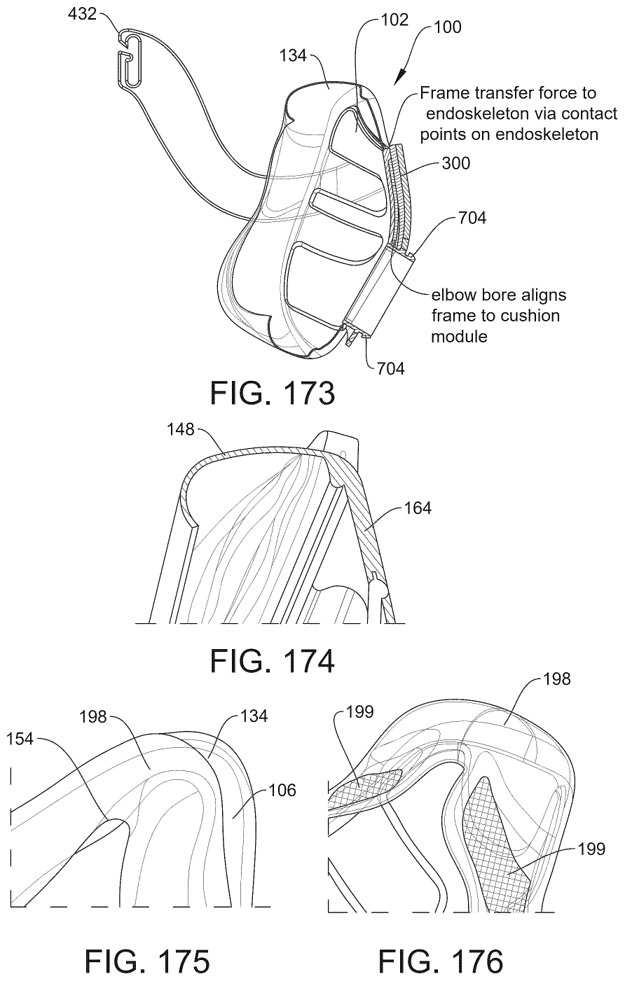

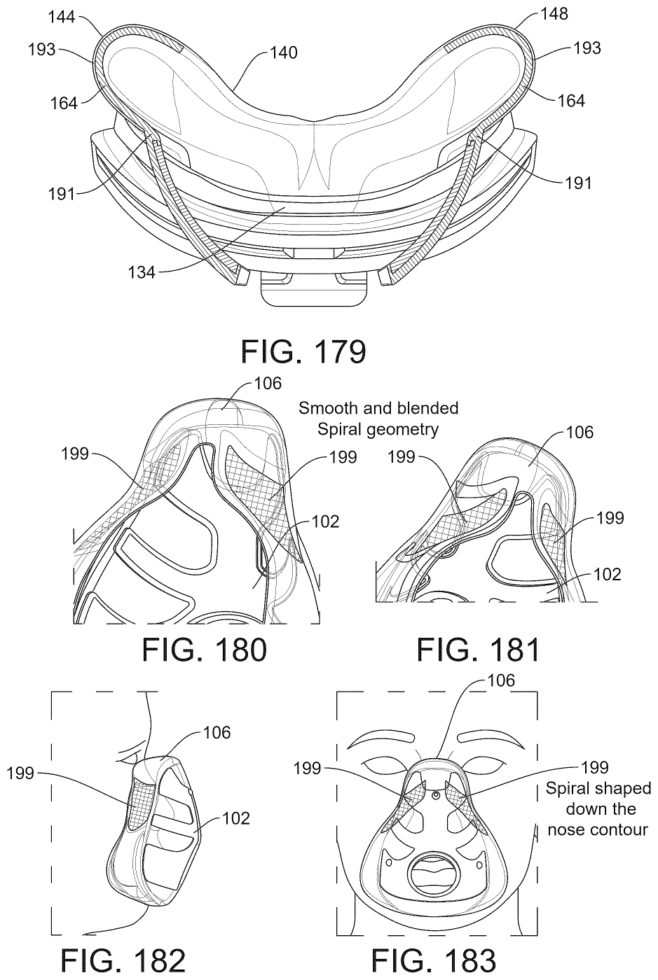

FIG. 173 illustrates a side view of the just mask of FIG. 172 (without the headgear) showing that the elbow bore can align to the frame attached to the endoskeleton-cushion module. The upper part of the frame can advantageously transfer force to the endoskeleton-cushion module via contact points on the endoskeleton. FIG. 174 illustrates the schematic consideration of the cushion curvature, thickness, angle and length required at the bottom corners 148 of the lip for the mask where the mask must not leak both when the wearer's stationary and with foreseeable dynamic movements. FIGS. 175 and 176 illustrates a zoomed-in view of the bubble nose bridge 198 having a spiral 199 thickened side of the nose section to optimise load distribution and compliance along nose contour for certain wearer face shapes, and improving comfort. The bubble nose bridge 198 can provide more space around the nose, reducing pressure and friction against the skin. This can make the mask more comfortable to wear for extended periods. With extra space around the nose, there is more room for air to flow within the mask. This can advantageously make breathing easier and reduce the feeling of suffocation that some people experience with tighter masks. The structural design of a bubble nose bridge 198 can help the mask conform better to the contours of the face. FIG. 177 illustrates a perspective view of another endoskeleton-cushion module have a whisker endoskeleton design, which is observed to have seal stability. FIG. 178 illustrates a zoomed-in view of a bottom corner of the cushion where the contour is smoothly curved in an eroteme or a question mark shape 193 and FIG. 179 illustrates a mask with eroteme or a question mark shape matching benchmark mask. The advantage of the eroteme shaped cushion 193 around the bottom corners of the mask 144, 148 is so as to better maintain the circular profile shape while adjusting for narrow and wide face shapes. The apex region 191 of the eroteme shape 193 is for allowing the pivoting of the mouth region and to flex about the apex for improving seal compliance for a wider mouth shape.

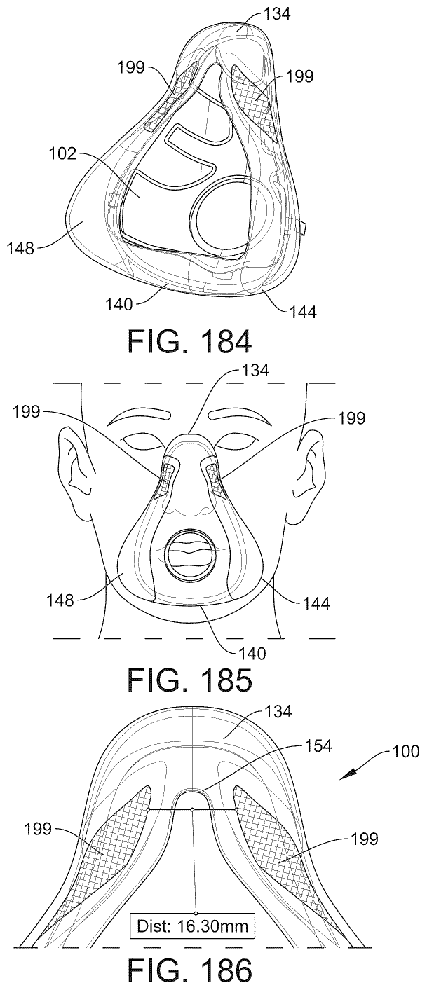

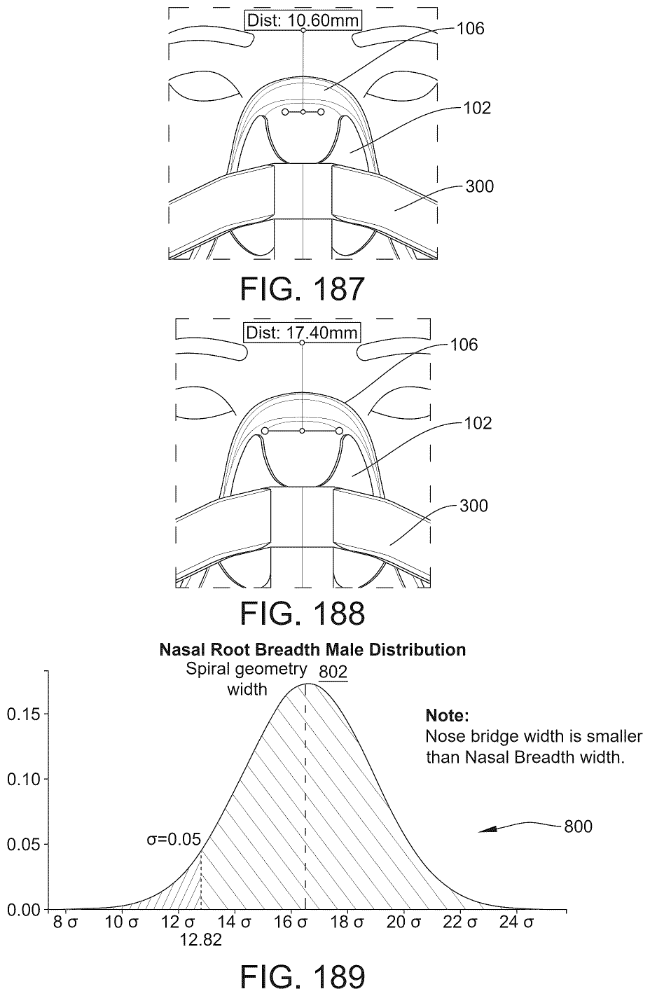

FIGS. 180 and 181 illustrates partial view of a smoothed and blended spiral 199 of an endoskeleton-cushion module, showing the blended spiral 199 having a predetermined thickness and its preferred geometrical positioning along the side of the nose for both nose sides. FIG. 182 illustrates a side view of a wearer of the endoskeleton-cushion module of FIG. 180 or 181. FIG. 183 illustrates a front view of the wearer of the endoskeleton-cushion module of FIG. 182, which shows a good and comfortable fit. FIG. 184 illustrates a perspective view of the endoskeleton-cushion module of FIG. 180 or 181. The advantage of having the spiral membrane region 199 at this position is that it can apply seal force along the nose contour of the wearer's face, and can deform in directions that matches the face shape of the mask that it needs to seal against. The spiral membrane regions 199 are advantageously intended to better concentrate the seal force to the side of the nose where air leakage is commonly experienced. Conventional membranes have thick regions that do not correspond in shape to the nose contour of the user. The approach for the spiral membrane regions 199 is to size for nominal nose width, which can deform for extremely tall and narrow nose shapes, while having a short nose width or wide nose width may each form a seal with the thin membrane edges which may curl. This minimises discomfort for shorter and wider nose bridges. FIG. 185 illustrates a front transparent schematic showing current membrane or cushion transitionally shaped across the nose contour, and wherein the spiral shape positioned at the side of the nose provide a comfortable fit to a nose contour that can be geometrically changed rapidly. The spiral membrane region is shown in this Figure. FIG. 186 illustrates a zoomed in view of the nose bridge contour region of a cushion showing the preferred distance between the left spiral membrane and the right spiral membrane positioned at the left side of the nose and the right side of the nose respectively. FIG. 187 illustrates a zoomed-in partial front view of the endoskeleton-cushion attached to a frame, showing a preferred distance between the left spiral membrane and the right spiral membrane contoured to curve up for better positioning at the left side of the nose and to the right side of the nose respectively to accommodate a taller and narrower nose shape, and FIG. 188 illustrates a zoomed-in partial front view of the endoskeleton-cushion attached to a frame, showing another preferred distance, which is longer compared to the distance of the mask as shown in FIG. 187, between the left spiral membrane and the right spiral membrane contoured to curve up for better positioning at the left side of the nose and the right side of the nose respectively to accommodate a short and wider nose shape. It may be appreciated that different ethnicities have different nose geometries and it is an advantage to create a mask that is more suited and more comfortable to accommodate different shape types.

FIG. 189 illustrates a graph 800 showing the nasal root breadth male distribution 802, showing one of the many anthropometric measurements used in physical anthropology to study human physical variation for facial reconstructions and for designing products like respiratory equipment. Regarding the distribution of nasal root breadth among males, it's important to note that such measurements can vary significantly across different populations and individuals due to genetic, environmental, and developmental factors. While the spiral geometry width can be adjusted for a bespoke mask such that different nose roots widths, which is the area where the nose meets the forehead, can be uniquely accommodated.

### Below Shows a Table of Male US Data

| Mean | Std Dev | Skewness | Kurtosis | Min | Max |
|---|---|---|---|---|---|
| 16.600 | 2.300 | 0.196 | .223 | 10.000 | 29.000 |

### Below Shows a Table of Female US Data

| Mean | Std Dev | Skewness | Kurtosis | Min | Max |
|---|---|---|---|---|---|
| 16.300 | 2.000 | 0.033 | .225 | 10.000 | 25.000 |

Males typically have a more pronounced nasal root depth compared to females. This means that the area of the nose where it joins the forehead may be deeper set or more prominent in males, contributing to a more angular or pronounced nasal profile. Females, tend to a have a shallower nasal root depth, contributing to a smoother transition from the forehead to the nose and a less pronounced nasal bridge. It may be appreciated that people are unique and different, and that these differences are general trends and there can be significant overlap between individuals of different sexes.

Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrial applicable.

What is claimed is:

1. A mask adapted for delivering pressurised air to a user, the mask comprising:
an endoskeleton of a first resilient material encapsulated to a lamina of a second resilient material forming a mask body with a cavity, wherein the second resilient material is relatively more flexible than the first resilient material;
the lamina having a face contactable skirt portion extending away from the endoskeleton perimeter, wherein the skirt portion comprises an outer layer and an inner layer for providing variable contoured thickness to outer lamina segments of the face contactable portion;
the face contactable skirt portion having a flexible cavity edge defining a cavity opening for receiving a nasal region, wherein the endoskeleton and lamina has an aperture, wherein the aperture is adapted to connect to an air delivery tube;
the thinner flexible cavity edge is adapted to wrap into the cavity, wherein when wrapped, the contoured thicker lamina segments wraps and seals around the sides of the nose.

2. The mask according to claim 1, wherein the cavity opening can further receive a mouth region, wherein the thinner flexible cavity edge at the bottom region has a raised edge, wherein the raised edge wraps and seals around the chin and cheek.

3. The mask according to claim 2, wherein the endoskeleton has a major segment and at least one minor segments.

4. The mask according to claim 3, wherein the major segment and the minor segment are of the same material, and wherein the at least one minor segments are hingedly connected to the major segment.

5. The mask according to claim 1, wherein the face contactable portion comprises a nose bridge centre segment, a left and right side of the nose segment, an upper lip segment positioned between a left and right bottom corner segment, and a left and right-side cheek segment.

6. The mask according to claim 4, wherein the face contactable portion comprises a nose bridge centre segment, a left and right side of the nose segment, a chin segment, a left and right bottom corner segment, and a left and right side cheek segment.

7. The mask according to claim 6, wherein the left-side nose bridge segment has a first thickened rib, and wherein the right-side nose bridge segment has a second thickened rib, wherein the first and second thickened ribs maintains its shape and seal at the nose bridge segment when subjected to pressurised air delivered by the air delivery tube.

8. The mask according to claim 7, wherein the flexible cavity edge between the left and right-side nose segment via the nasal centre segment comprises a biased contour connecting between a left-side nose wing and a right-side nose wing, wherein the biased contour allows for an increased seal with the nasal bridge while the nose wings increase surface area allows for reducing air blow out from sides of nose.

9. The mask according to claim 8, wherein the biased contour is a stress relief radius.

10. The mask according to claim 9, wherein the flexible cavity edge is a polished edge around the edge of the face contacting portion, wherein the polished edge is greater than 3 mm.

11. The mask according to claim 10, wherein the left and right-side nose segments each comprises a contoured segment thicker relative to the nose bridge centre segment.

12. The mask according to claim 11, wherein the skirt portion comprises an inner seal lamina adjoining from a first partial section of the left-side nose segment and a second partial section of the right-side nose segment via the nose bridge centre segment.

13. The mask according to claim 12, wherein the left-side cheek segment and the right-side cheek segment each comprises stiffening ribs for maintaining the shape of the skirt portion when subjected to pressurised air delivered by the air delivery tube.

14. The mask according to claim 13, wherein the outer lamina of the face contactable skirt portion and a cantilever inner lamina each extending from the endoskeleton perimeter at the same location, wherein the cantilever inner lamina has a predetermined thickness.

15. The mask according to claim 14, wherein the outer lamina is frosted, and wherein the outer lamina is made of silicone.

16. The mask according to claim 15, further comprising a chin gimble.

17. The mask according to claim 16, wherein the flexible cavity edge comprises an extended chin lamina for reducing jaw leak at the chin.

18. The mask according to claim 17, wherein a top profile of the skirt portion of the chin region has a ‘V’ shape for allowing the chin region of the skirt portion contact the chin earlier, when in use.

19. The mask according to claim 18, wherein the chin region has a thickness between 0.20 to 0.45 mm thin lamina for reducing force on chin and to accommodate mouth movement.

20. The mask according to claim 18, wherein the nasal bridge region has a thickness between 0.20 to 0.45 mm thin lamina for reducing force on centre and/or side of the nasal bridge.

21. The mask according to claim 1, wherein the endoskeleton is made from a semi-rigid plastic material selected from the group of: polycarbonate, polypropylene, polyester, Acrylonitrile Butadiene Styrene (ABS), thermoplastic elastomer (TPE), polycarbonate-ABS blend, polyethylene and polyamide (nylon), wherein the plastic material of the endoskeleton is relatively rigid compared to the lamina.

22. The mask according to claim 1, further comprising an exoskeleton securable to the encapsulated endoskeleton, the exoskeleton having an aperture with a shape congruent to the aperture of the encapsulated endoskeleton, wherein when secured, the exoskeleton aperture is positioned over the encapsulated endoskeleton aperture, such that the air delivery tube is connectable to the cavity through the exoskeleton aperture and the encapsulated endoskeleton aperture.

23. The mask according to claim 22, wherein the air delivery tube having a first end and a second end, wherein the first end has a snap connection for sealingly interlocking to the exoskeleton aperture and the encapsulated endoskeleton aperture, and wherein the second end is attachable to a Continuous Positive Airway Pressure (CPAP) apparatus.

24. The mask according to claim 23, wherein the first end of the air delivery tube comprises a snap release mechanism positioned inside the exoskeleton, wherein the mechanism of release is facilitated by rotating about a fulcrum and deforming an elastic thermoplastic elastomer, which loosens and allows rotation of the first end of the delivery tube relative to the endoskeleton aperture to disengage.

25. The mask according to claim 24, wherein the air delivery tube further comprises an anti-asphyxia valve (AAV) in an air inflow channel positioned between the first end and the second end of the air delivery tube.

26. The mask according to claim 25, wherein the air delivery tube comprises a CO₂ washout channel, which is an independent channel to the inflow of air from the second end to the first end.

27. The mask according to claim 26, wherein the air delivery tube comprises annular vent holes for directing air from the CO₂ washout channel in a conical shape out of the air delivery tube.

28. The mask according to claim 27, wherein the endoskeleton comprises an encapsulated area and a non-encapsulated area, wherein a vent hole array is positioned at the non-encapsulated area.

29. The mask according to claim 28, wherein the vent hole array has a profile that is at least one selected from the group of: front facing, angled out to the side, tapered, and non-tapered.

30. The mask according to claim 5, wherein the encapsulated endoskeleton has a major segment of the first resilient material, and minor segments of the second resilient and flexible material, wherein the major segment has elongate structural members for providing support to the face contactable skirt portion of at least one segment selected from the group of: the left and right side of the nose segment, the upper lip segment, the left and right bottom corner segment, and the left and right-side cheek segment.

31. The mask according to claim 30, wherein an exoskeleton is mountable to the front of the endoskeleton; a first frame arm having a first exoskeleton connector securable to a first side of the exoskeleton; a second frame arm having a second exoskeleton connector securable to a second side of the exoskeleton; wherein each of the first frame arm and the second frame arm bifurcates at a middle frame segment forming an upper frame arm and a lower frame arm, wherein the upper frame arm has an upper headgear connector for securing an upper headgear strap, and wherein the lower frame arm has a lower headgear connector for securing a lower headgear strap.

32. The mask according to claim 31, wherein the first exoskeleton connector and the second exoskeleton connector are each angled between 15° to 25° with respect to the longitudinal axis of the frame arm.

33. The mask according to claim 32, wherein each of the frame arms have vertically corrugated portions positioned along the longitudinal axis of the frame arm between the connecting ends.

34. The mask according to claim 33, wherein a non-corrugated portion is positioned between the corrugated portion and the upper headgear strap securing means, wherein the non-corrugated portion of a resiliently stiff material is adapted to reduce twisting of the frame arm.

35. The mask according to claim 31, wherein the frame arms are of a resiliently stiff material.

36. The mask according to claim 31, wherein the frame arm has a first rotated corrugated portion, and the lower frame arm having a second rotated corrugated portion, wherein the first rotated corrugated portion is angled differently to the second rotated corrugated portion.

37. The mask according to claim 36, wherein a border of resilient material is positioned around the first rotated corrugated portion of the upper frame arm.

38. The mask according to claim 37, wherein the lower frame arm has a bevelled hook for receiving and securing a lower headgear clip or wherein the lower headgear clip has a bevelled hook for receiving and securing the lower frame arm, wherein the bevelled hook has a bevelled edge.

39. The mask according to claim 38, wherein the secured lower headgear clip is disengageable from the bevelled hook by pivotally rotating the lower headgear clip out of the hook such that the clip is parallel to a first bevelled edge of the bevelled hook.

40. The mask according to claim 39, wherein the bevelled hook further comprises a second bevelled edge angled 90° relative to the first bevelled edge, wherein the secured lower headgear clip is disengageable from the bevelled hook by either pivotally rotating the lower headgear clip out of the hook such that the clip is parallel to the first bevelled edge or the second bevelled edge of the bevelled hook.

41. The mask according to claim 31, wherein the headgear comprises a raised base panel of a resilient flexible material connecting between curved bottom headgear straps for travelling around ears of a user.

42. The mask according to claim 41, wherein an interior of the curved bottom headgear straps each having a structured polypropylene sheet flanking the raised base panel for providing a relatively rigid structured area, and a soft material encapsulating the structured element of the bottom headgear strap for providing a relatively moderate elasticity to the lower headgear strap.

43. The mask according to claim 42, wherein an exterior of the upper and lower headgear straps comprises a hook and loop fastener tab at the distal end of the straps for preventing clip slippage.

44. The mask according to claim 31, further comprising an elastic tether securable to a lower headgear clip at one end and an elastic loop portion at the other end, wherein the exoskeleton has a hook for receiving and securing the elastic loop portion and the clip.

45. The mask according to claim 22, wherein the exoskeleton has an upper frame portion positioned between the exoskeleton aperture and an upper headgear fastening member, wherein the upper frame portion comprises a vertical corrugated section partially along the length of the upper frame portion.

46. The mask according to claim 45, wherein the upper frame portion further comprises a hinge portion positioned between the exoskeleton aperture and the vertical corrugated section.

47. The mask according to claim 22, wherein the exoskeleton has an upper frame portion positioned between the exoskeleton aperture an upper headgear fastening member, wherein the exoskeleton comprises at least one corrugated portion.

48. The mask according to claim 47, wherein the upper frame portion comprises a corrugated portion partially along the length of the upper frame portion, wherein the profile of the corrugated portion is angled with respect to the vertical axis of the mask, wherein the upper frame portion comprises a second corrugated portion spaced apart from the corrugated portion.

49. The mask according to claim 22, wherein the exoskeleton is corrugated.

50. The mask according to claim 49, wherein the profile of the corrugated portion is one selected from the group of: sinusoidal, elliptical, triangular, square, and rectangular.

51. The mask according to claim 50, wherein the corrugated portion has a wall section of a consistent width or varying widths along the length of the exoskeleton.

52. The mask according to claim 22, wherein the encapsulated endoskeleton aperture has a non-circular shape profile, wherein the perimeter defining the endoskeleton aperture has one or more frame retention means for interlocking with the back of the exoskeleton.

53. The mask according to any one of claim 52, wherein the front of the exoskeleton has one or more air delivery tube retention means for interlocking with the air delivery tube.

54. The mask according to claim 22, wherein the encapsulated endoskeleton aperture has a non-circular shape profile, wherein the perimeter defining the endoskeleton aperture has one or more air delivery tube retention means for interlocking with the air delivery tube, and wherein the endoskeleton comprises one or more frame retention means for interlocking with the back of the exoskeleton.

55. The mask according to claim 22, wherein the exoskeleton has an upper frame portion positioned between the exoskeleton aperture and an upper headgear fastening member, wherein the upper frame portion comprises a first hinge portion spaced away from a second hinge portion, wherein the hinge portions are partially along the length of the upper frame portion.

56. The mask according to claim 18, further comprising an exoskeleton headgear module mountable to the endoskeleton, wherein the exoskeleton headgear module comprises a fastening device for connecting the exoskeleton with a headgear, wherein the fastening device has an elastic memory material connected between an exoskeleton fastening member and a headgear fastening member for securing the fastening members together.

57. The mask according to claim 56, wherein providing a pulling force to the headgear away from the exoskeleton disengages the fastening members and stretches the elastic memory material and wherein when the pulling force is released, the elastic memory material moves the fastening members towards each other and self-secures the fastening members together.

58. The mask according to claim 18, further comprising a headgear module with at least one stiffening element.

59. The mask according to claim 58, wherein the headgear module comprises an upper headgear strap and a lower headgear strap, wherein the lower headgear strap comprises a stiffening element such that the lower headgear strap is forward biasing towards the exoskeleton.

60. The mask according to claim 59, wherein the stiffening element is one chosen from the group of: heat staking resilient materials, a glued resilient material, a stitching of a stiff material, a thicker fabric, a thermoformed shape, a stitching of a hook and loop fastener portion.

61. The mask according to claim 11, wherein the contoured segment of the left and right-side nose segments each have a membrane of a predetermined spiral shape for thickening predetermined portions of the lamina along the nose section.

62. The mask according to claim 23, wherein the first end of the air delivery tube comprises a snap release mechanism for interlocking to the endoskeleton or to the exoskeleton.

63. The mask according to claim 31, wherein an exoskeleton is mountable to the front of the endoskeleton with at least three loading points, wherein the first loading point is positioned proximal to the nose bridge region, wherein the second loading point is positioned proximal to the left bottom corner segment, and wherein the third loading point is positioned proximal to the right bottom corner segment.
